SEQUENCE LISTING (175 sequences)

SEQ ID NO: 1 - DNA, 348 bp, Homo sapiens
gaggtgcagctgttggagtctgggggaggcttggtacagcctggggggtccctgcgtctctcctgtgcagcctccggattcacctttagcagctatgccatgagctgggtccgccaggctccagggaagggtctagagtgggtctcagctattagtggtagtggtggtagcacatactacgcagactccgtgaagggccggttcaccatctcccgtgacaattccaagaacacgctgtatctgcaaatgaacagcctgcgtgccgaggacaccgcggtatattactgtgcgaaaagttatggtgcttttgactactggggccagggaaccctggtcaccgtctcgagc

SEQ ID NO: 2 - protein, 116 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSYGAFDYWGQGTLVTVSS

SEQ ID NO: 3 - DNA, 360 bp, Homo sapiens
  misc_feature (307)..(307): N=G, A, T or C
gaggtgcagctgttggagtctgggggaggcttggtacagcctggggggtccctgcgtctctcctgtgcagcctccggattcacctttagcagctatgccatgagctgggtccgccaggctccagggaagggtctagagtgggtctcagctattagtggtagtggtggtagcacatactacgcagactccgtgaagggccggttcaccatctcccgtgacaattccaagaacacgctgtatctgcaaatgaacagcctgcgtgccgaggacaccgcggtatattactgtgcgaaaagttatggtgctnnknnknnknnktttgactactggggccagggaaccctggtcaccgtctcgagc

SEQ ID NO: 4 - protein, 120 aa, Homo sapiens
  MISC_FEATURE (103)..(103): X = any amino acid
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSYGAXXXXFDYWGQGTLVTVSS

SEQ ID NO: 5 - DNA, 372 bp, Homo sapiens
gacatccagatgacccagtctccatcctccctgtctgcatctgtaggagaccgtgtcaccatcacttgccgggcaagtcagagcattagcagctatctggcacagtggtagtgaacggcccgttcagtctcgtaatcgtcgatattaaattggtaccagcagaaaccagggaaagcccctaagctcctgatctatgctgcatccagtttgcaaagtggggtcccatcacgtttcagtggcagtggatctgggacagatttcactctcaccatcagcagtctgcaacctgaagattttgctacgtactactgtcaacagagttacagtacccctaatacgttcggccaagggaccaaggtggaaatcaaacgg

SEQ ID NO: 6 - protein, 108 aa, Homo sapiens
DIQMTQSPSSLSASVGDRVTITCRASQSISSYLNWYQQKPGKAPKLLIYAASSLQSGVPSRFSGSGSGTDFTLTISSLQPEDFATYYCQQSYSTPNTFGQGTKVEIKR

SEQ ID NO: 7 - DNA, 324 bp, Artificial sequence
  Sequence selected from diversified human antibody sequence
gacatccagatgacccagtctccatcctccctgtctgcatctgtaggagaccgtgtcaccatcacttgccgggcaagtcagagcattattaagcatttaaagtggtaccagcagaaaccagggaaagcccctaagctcctgatctatggtgcatcccggttgcaaagtggggtcccatcacgtttcagtggcagtggatctgggacagatttcactctcaccatcagcagtctgcaacctgaagattttgctacgtactactgtcaacagggggctcggtggcctcagacgttcggccaagggaccaaggtggaaatcaaacgg

SEQ ID NO: 8 - protein, 108 aa, Artificial sequence
  Sequence selected from diversified human antibody sequence
DIQMTQSPSSLSASVGDRVTITCRASQSIIKHLKWYQQKPGKAPKLLIYGASRLQSGVPSRFSGSGSGTDFTLTISSLQPEDFATYYCQQGARWPQTFGQGTKVEIKR

SEQ ID NO: 9 - DNA, 324 bp, Artificial sequence
  Sequence selected from diversified human antibody sequence
gacatccagatgacccagtctccatcctccctgtctgcatctgtaggagaccgtgtcaccatcacttgccgggcaagtcagagcatttattatcatttaaagtggtaccagcagaaaccagggaaagcccctaagctcctgatctataaggcatccacgttgcaaagtggggtcccatcacgtttcagtggcagtggatctgggacagatttcactctcaccatcagcagtctgcaacctgaagattttgctacgtactactgtcaacaggttcggaaggtgcctcggacgttcggccaagggaccaaggtggaaatcaaacgg

SEQ ID NO: 10 - protein, 108 aa, Artificial sequence
  Sequence selected from diversified human antibody sequence
DIQMTQSPSSLSASVGDRVTITCRASQSIYYHLKWYQQKPGKAPKLLIYKASTLQSGVPSRFSGSGSGTDFTLTISSLQPEDFATYYCQQVRKVPRTFGQGTKVEIKR

SEQ ID NO: 11 - DNA, 360 bp, Artificial sequence
  Sequence selected from diversified human antibody sequence
gaggtgcagctgttggagtctgggggaggcttggtacagcctggggggtccctgcgtctctcctgtgcagcctccggatttaggattagcgatgaggatatgggctgggtccgccaggctccagggaagggtctagagtgggtatcaagcatttatggccctagcggtagcacatactacgcagactccgtgaagggccggttcaccatctcccgtgacaattccaagaacacgctgtatctgcaaatgaacagcctgcgtgccgaggacaccgcggtatattattgcgcgagtgctttggagccgctttcggagcccctgggcttttggggtcagggaaccctggtcaccgtctcgagc

SEQ ID NO: 12 - protein, 120 aa, Artificial sequence
  Sequence selected from diversified human antibody sequences
EVQLLESGGGLVQPGGSLRLSCAASGFRISDEDMGWVRQAPGKGLEWVSSIYGPSGSTYYADSVKGRFTISRDNSKNTLYLQMNSLRAEDTAVYYCASALEPLSEPLGFWGQGTLVTVSS

SEQ ID NO: 13 - DNA, 348 bp, Artificial sequence
  Sequence selected from diversified human antibody sequences.
gaggtgcagctgttggagtctgggggaggcttggtacagcctggggggtccctgcgtctctcctgtgcagcctccggattcacctttgatctttataatatgttttgggtccgccaggctccagggaagggtctagagtgggtctcatttattagtcagactggtaggcttacatggtacgcagactccgtgaagggccggttcaccatctcccgcgacaattccaagaacacgctgtatctgcaaatgaacagcctgcgtgccgaggacaccgcggtatattactgtgcgaaaacgctggaggattttgactactggggccagggaaccctggtcaccgtctcgagc

SEQ ID NO: 14 - protein, 116 aa, Artificial sequence
  Sequence selected from diversified human antibody sequences.
EVQLLESGGGLVQPGGSLRLSCAASGFTFDLYNMFWVRQAPGKGLEWVSFISQTGRLTWYADSVKGRFTISRDNSKNTLYLQMNSLRAEDTAVYYCAKTLEDFDYWGQGTLVTVSS

SEQ ID NO: 15 - DNA, 324 bp, Artificial sequence
  Sequence selected from diversified human antibody sequences.
gacatccagatgacccagtctccatcctccctgtctgcatctgtaggagaccgtgtcaccatcacttgccgggcaagtcagagcgttaaggagtttttatggtggtaccagcagaaaccagggaaagcccctaagctcctgatctatatggcatccaatttgcaaagtggggtcccatcacgtttcagtggcagtggatctgggacagatttcactctcaccatcagcagtctgcaacctgaagattttgctacgtactactgtcaacagaagtttaagctgcctcgtacgttcggccaagggaccaaggtggaaatcaaacgg

SEQ ID NO: 16 - protein, 108 aa, Artificial sequence
  Sequence selected from diversified human antibody sequences.
DIQMTQSPSSLSASVGDRVTITCRASQSVKEFLWWYQQKPGKAPKLLIYMASNLQSGVPSRFSGSGSGTDFTLTISSLQPEDFATYYCQQKFKLPRTFGQGTKVEIKR

SEQ ID NO: 17 - DNA, 324 bp, Artificial sequence
  Sequence selected from diversified human antibody sequences.
gacatccagatgacccagtctccatcctctctgtctgcatctgtaggagaccgtgtcaccatcacttgccgggcaagtcagagcattgatagttatttacattggtaccagcagaaaccagggaaagcccctaagctcctgatctatagtgcatccgagttgcaaagtggggtcccatcacgtttcagtggcagtggatctgggacagatttcactctcaccatcagcagtctgcaacctgaagattttgctacgtactactgtcaacaggttgtgtggcgtccttttacgttcggccaagggaccaaggtggaaatcaaacgc

SEQ ID NO: 18 - protein, 108 aa, Artificial sequence
  Sequence selected from diversified human antibody sequences.
DIQMTQSPSSLSASVGDRVTITCRASQSIDSYLHWYQQKPGKAPKLLIYSASELQSGVPSRFSGSGSGTDFTLTISSLQPEDFATYYCQQVVWRPFTFGQGTKVEIKR

SEQ ID NO: 19 - DNA, 324 bp, Artificial sequence
  Sequence selected from diversified human antibody sequences.
gacatccagatgacccagtctccatcctccctgtctgcatctgtaggagaccgtgtcaccatcacttgccgggcaagtcagagcatttttatgaatttattgtggtaccagcagaaaccagggaaagcccctaagctcctgatctataatgcatccgtgttgcaaagtggggtcccatcacgtttcagtggcagtggatctgggacagatttcactctcaccatcagcagtctgcaacctgaagattttgctacgtactactgtcaacaggttgtgtggcgtccttttacgttcggccaagggaccaaggtggaaatcaaacgg

SEQ ID NO: 20 - protein, 108 aa, Artificial sequence
  Sequence selected from diversified human antibody sequences.
DIQMTQSPSSLSASVGDRVTITCRASQSIFMNLLWYQQKPGKAPKLLIYNASVLQSGVPSRFSGSGSGTDFTLTISSLQPEDFATYYCQQVVWRPFTFGQGTKVEIKR

SEQ ID NO: 21 - DNA, 324 bp, Artificial sequence
  Sequence selected from diversified human antibody sequences.
gacatccagatgacccagtctccatcctccctgtctgcatctgtaggagaccgtgtcaccatcacttgccgggcaagtcagagcatttatgatgcgttagagtggtaccagcagaaaccagggaaagcccctaagctcctgatctatactgcatcccggttgcaaagtggggtcccatcacgtttcagtggcagtggatctgggacagatttcactctcaccatcagcagtctgcaacctgaagattttgctacgtactactgtcaacaggttatgcagcgtcctgttacgttcggccaagggaccaaggtggaaatcaaacgg

SEQ ID NO: 22 - protein, 108 aa, Artificial sequence
  Sequence selected from diversified human antibody sequences.
DIQMTQSPSSLSASVGDRVTITCRASQSIYDALEWYQQKPGKAPKLLIYTASRLQSGVPSRFSGSGSGTDFTLTISSLQPEDFATYYCQQVMQRPVTFGQGTKVEIKR

SEQ ID NO: 23 - DNA, 324 bp, Artificial sequence
  Sequence selected from diversified human antibody sequences.
gacatccagatgacccagtctccatcctccctgtctgcatctgtaggagaccgtgtcaccatcacttgccgggcaagtcagagcatttatgatgctttacagtggtaccagcagaaaccagggaaagcccctaagctcctgatctatactgcatcccggttgcaaagtggggtcccatcacgtttcagtggcagtggatctgggacagatttcactctcaccatcagcagtctgcaacctgaagattttgctacgtaccactgtcaacaggttatgcagcgtcctgttacgttcggccaagggaccaaggtggaaatcaaacgg

SEQ ID NO: 24 - protein, 107 aa, Artificial sequence
  Sequence selected from diversified human antibody sequences.
DIQMTQSPSSLSASVGDRVTITCRASQSIYDALWYQQKPGKAPKLLIYTASRLQSGVPSRFSGSGSGTDFTLTISSLQPEDFATYHCQQVMQRPVTFGQGTKVEIKR

SEQ ID NO: 25 - DNA, 324 bp, Artificial sequence; identical to SEQ ID NO: 15
  Sequence selected from diversified hauman antibody sequences.

SEQ ID NO: 26 - protein, 108 aa, Artificial sequence; identical to SEQ ID NO: 16
  Sequence selected from diversified human antibody sequences.

SEQ ID NO: 27 - DNA, 324 bp, Artificial sequence
  Sequence selected from diversified human antibody sequences.
gacatccagatgacccagtctccatcctccctgtctgcatctgtaggagaccgtgtcaccatcacttgccgggcaagtcagagcatttggacgaagttacattggtaccagcagaaaccagggaaagcccctaagctcctgatctatatggcatccagtttgcaaagtggggtcccatcacgtttcagtggcagtggatctgggacagatttcactctcaccatcagcagtctgcaacctgaagattttgctacgtactactgtcaacagtggtttagtaatcctagtacgttcggccaagggaccaaggtggaaatcaaacgc

SEQ ID NO: 28 - protein, 108 aa, Artificial sequence
  Sequence selected from diversified human antibody sequences.
DIQMTQSPSSLSASVGDRVTITCRASQSIWTKLHWYQQKPGKAPKLLIYMASSLQSGVPSRFSGSGSGTDFTLTISSLQPEDFATYYCQQWFSNPSTFGQGTKVEIKR

SEQ ID NO: 29 - DNA, 324 bp, Artificial sequence
  Sequence selected from diversified human antibody sequences.
gacatccagatgacccagtctccatcctccctgtctgcatctgtaggagaccgtgtcaccatcacttgccgggcaagtcagagcatttagccgattttatgttggtaccagcagaaaccagggaaagcccctaagctcctgatctatgctgcatccagtttgcaaagtggggtcccatcacgtttcagtggcagtggatctgggacagatttcactctcaccatcagcagtctgcaacctgaagattttgctacgtactactgtcaacagattcagcatattcctgtgacgttcggccaagggaccaaggtggaaatcaaacgg

SEQ ID NO: 30 - protein, 107 aa, Artificial sequence
  Sequence selected from diversified human antibody sequences.
DIQMTQSPSSLSASVGDRVTITCRASQSIPILCWYQQKPGKAPKLLIYAASSLQSGVPSRFSGSGSGTDFTLTISSLQPEDFATYYCQQIQHIPVTFGQGTKVEIKR

SEQ ID NO: 31 - DNA, 324 bp, Artificial sequence
  Sequence selected from diversified human antibody sequences.
gacatccagatgacccagtctccatcctccctgtctgcatctgtaggagaccgtgtcaccatcacttgccgggcaagtcagagcattgggtaggatttacattggtaccagcagaaaccagggaaagcccctaagctcctgatctatacggcatcccttttgcaaagtggggtcccatcacgtttcagtggcagtggatctgggacagatttcactctcaccatcagcagtctgcaacctgaagattttgctacgtactactgtcaacagcagagtgcttttcctaatacgctcggccaagggaccaaggtggaaatcaaacgg

SEQ ID NO: 32 - protein, 107 aa, Artificial sequence
  Sequence selected from diversified human antibody sequences.
DIQMTQSPSSLSASVGDRVTITCRASQSIGDLHWYQQKPGKAPKLLIYTASLLQSGVPSRFSGSGSGTDFTLTISSLQPEDFATYYCQQQSAFPNTLGQGTKVEIKR

SEQ ID NO: 33 - DNA, 324 bp, Artificial sequence
  Sequence selected from diversified human antibody sequences.
gacatccagatgacccagtctccatcctccctgtctgcatccgtaggagaccgtgtcaccatcacttgccgggcaagtcagagcataacgaagaatttactttggtaccagcagaaaccagggaaagcccctaagctcctgatctattaggcatcctctttgcaaagtggggtcccatcacgtttcagtggcagtggatctgggacagatttcactctcaccatcagcagtctgcaacctgaagattttgctacgtactactgtcaacagcttcgtcataagcctccgacgttcggccaagggaccaaggtggaaatcaaacgg

SEQ ID NO: 34 - protein, 107 aa, Artificial sequence
  Sequence selected from diversified human antibody sequences.
DIQMTQSPSSLSASVGDRVTITCRASQSITKNLLWYQQKPGKAPKLLIYASSLQSGVPSRFSGSGSGTDFTLTISSLQPEDFATYYCQQLRHKPPTFGQGTKVEIKR

SEQ ID NO: 35 - DNA, 324 bp, Artificial sequence
  Sequence selected from diversified human antibody sequence.
gacatccagatgacccagtctccatcctccctgtctgcatctgtaggagaccgtgtcaccatcacttgccgggcaagtcagagcatttagaagtctttaaggtggtaccagcagaaaccagggaaagcccctaagctcctgatctatcatgcatccgatttgcaaagtggggtcccatcacgtttcagtggcagtggatctgggacagatttcactctcaccatcagcagtctgcaacctgaagattttgctacgtactactgtcaacagatggttaatagtcctgttacgttcggccaagggaccaaggtggaaatcaaacgg

SEQ ID NO: 36 - protein, 107 aa, Artificial sequence
  Sequence selected from divedsified human antibody sequences.
DIQMTQSPSSLSASVGDRVTITCRASQSIKSLRWYQQKPGKAPKLLIYHASDLQSGVPSRFSGSGSGTDFTLTISSLQPEDFATYYCQQMVNSPVTFGQGTKVEIKR

SEQ ID NO: 37 - DNA, 324 bp, Artificial sequence
  Sequence selected from diversified human antibody sequences.
gacatccagatgacccagtctccatcctccctgtctgcatctgtaggagaccgtgtcaccatcacttgccgggcaagtcagagcatttagacggcgttacattggtaccagcagaaaccagggaaagcccctaagctcctgatctattctgcatccagtttgcaaagtggggtcccatcacgtttcagtggcagtggatctgggacagatttcactctcaccatcagcagtctgcaacctgaagattttgctacgtactactgtcaacagtcgagttttttgccttttacgttcggccaagggaccaaggtggaaatcaaacgg

SEQ ID NO: 38 - protein, 107 aa, Artificial sequence
  Sequence selected from diversified human antibody sequences.
DIQMTQSPSSLSASVGDRVTITCRASQSITALHWYQQKPGKAPKLLIYSASSLQSGVPSRFSGSGSGTDFTLTISSLQPEDFATYYCQQSSFLPFTFGQGTKVEIKR

SEQ ID NO: 39 - DNA, 324 bp, Artificial sequence
  Sequence selected from diversified human antibody sequences.
gacatccagatgacccagtctccatcctccctgtctgcatctgtaggagaccgtgtcaccatcacttgccgggcaagtcagagcattgggccgaatttagagtggtaccagcagaaaccagggaaagcccctaagctcctgatctatgctgcatccagtttgcaaagtggggtcccatcacgtttcagtggcagtggatctgggacagatttcactctcaccatcagcagtctgcaacctgaagattttgctacgtactactgtcaacagcagatggggcgtcctcggacgttcggccaagggaccaaggtggaaatcaaacgg

SEQ ID NO: 40 - protein, 108 aa, Artificial sequence
  Sequence selected from diversified human antibody sequences.
DIQMTQSPSSLSASVGDRVTITCRASQSIGPNLEWYQQKPGKAPKLLIYAASSLQSGVPSRFSGSGSGTDFTLTISSLQPEDFATYYCQQQMGRPRTFGQGTKVEIKR

SEQ ID NO: 41 - DNA, 324 bp, Artificial sequence
  Sequence selected from diversified human antibody sequences.
gacatccagatgacccagtctccatcctccctgtctgcatctgtaggagaccgtgtcaccatcacttgccgggcaagtcagagcattaagcattagttagcttggtaccagcagaaaccagggaaagcccctaagctcctgatctataaggcatccgtgttgcaaagtggggtcccatcacgtttcagtggcagtggatctgggacagatttcactctcaccatcagcagtctgcaacctgaagattttgctacgtactactgtcaacagcttaggcgtcgtcctactacgttcggccaagggaccaaggtggaaatcaaacgg

SEQ ID NO: 42 - protein, 107 aa, Artificial sequence
  Sequence selected from diversified huamn antibody sequences.
DIQMTQSPSSLSASVGDRVTITCRASQSIKHLAWYQQKPGKAPKLLIYKASVLQSGVPSRFSGSGSGTDFTLTISSLQPEDFATYYCQQLRRRPTTFGQGTKVEIKR

SEQ ID NO: 43 - DNA, 324 bp, Artificial sequence
  Sequence selected from diversified human antibody sequences.
gacatccagatgacccagtctccatcctccctgtctgcatctgtaggagaccgtgtcaccatcacttgccgggcaagtcagagcgttaaggcttagttaacttggtaccagcagaaaccagggaaagcccctaagctcctgatctataaggcatccactttgcaaagtggggtcccatcacgtttcagtggcagtggatctgggacagatttcactctcaccatcagcagtctgcaacctgaagattttgctacgtactactgtcaacagcatagttctaggccttatacgttcggccaagggaccaaggtggaaatcaaacgg

SEQ ID NO: 44 - protein, 107 aa, Artificial sequence
  Sequence selected from diversified human antibody sequences.
DIQMTQSPSSLSASVGDRVTITCRASQSVKALTWYQQKPGKAPKLLIYKASTLQSGVPSRFSGSGSGTDFTLTISSLQPEDFATYYCQQHSSRPYTFGQGTKVEIKR

SEQ ID NO: 45 - DNA, 324 bp, Artificial sequence
  Sequence selected from diversified human antibody sequences.
gacatccagatgacccagtctccatcctccctgtctgcatctgtaggagaccgtgtcaccatcacttgccgggcaagtcagagcattgagaatcggttaggttggtaccagcagaaaccagggaaagcccctaagctcctgatctattaggcgtccttgttgcaaagtggggtcccatcacgtttcagtggcagtggatctgggacagatttcactctcaccatcagcagtctgcaacctgaagattttgctacgtactactgtcaacaggattcgtattttcctcgtacgttcggccaagggaccaaggtggaaatcaaacgg

SEQ ID NO: 46 - protein, 107 aa, Artificial sequence
  Sequence selected from diversified human antibody sequences.
DIQMTQSPSSLSASVGDRVTITCRASQSIENRLGWYQQKPGKAPKLLIYASLLQSGVPSRFSGSGSGTDFTLTISSLQPEDFATYYCQQDSYFPRTFGQGTKVEIKR

SEQ ID NO: 47 - DNA, 324 bp, Artificial sequence
  Sequence selected from diversified human antibody sequences.
gacatccagatgacccagtctccatcctccctgtctgcatctgtaggagaccgtgtcaccatcacttgccgggcaagtcagagcattatggataagttaaagtggtaccagcagaaaccagggaaagcccctaagctcctgatctattaggcatccattttgcaaagtggggtcccatcacgtttcagtggcagtggatctgggacagatttcactctcaccatcagcagtctgcaacctgaagattttgctacgtactactgtcaacaggatagtgggggtcctaatacgttcggccaagggaccaaggtggaaatcaaacgg

SEQ ID NO: 48 - protein, 107 aa, Artificial sequence
  Sequence selected from diversified human antibody sequences.
DIQMTQSPSSLSASVGDRVTITCRASQSIMDKLKWYQQKPGKAPKLLIYASILQSGVPSRFSGSGSGTDFTLTISSLQPEDFATYYCQQDSGGPNTFGQGTKVEIKR

SEQ ID NO: 49 - DNA, 324 bp, Artificial sequence
  Sequence selected from diversified human antibody sequences.
gacatccagatgacccagtctccatcctccctgtctgcatctgtaggagaccgtgtcaccatcacttgccgggcaagtcagagcattgggaggaatttagagtggtaccagcagaaaccagggaaagcccctaagctcctgatctatgatgcatcccatttgcaaagtggggtcccatcacgtttcagtggcagtggatctgggacagatttcactctcaccatcagcagtctgcaacctgaagattttgctacgtactactgtcaacagtcgcgttggcttcctcgtacgttcggccaagggaccaaggtggaaatcaaacgg

SEQ ID NO: 50 - protein, 108 aa, Artificial sequence
  Sequence selected from diversified human antibody sequences.
DIQMTQSPSSLSASVGDRVTITCRASQSIGRNLEWYQQKPGKAPKLLIYDASHLQSGVPSRFSGSGSGTDFTLTISSLQPEDFATYYCQQSRWLPRTFGQGTKVEIKR

SEQ ID NO: 51 - DNA, 324 bp, Artificial sequence
  Sequence selected from diversified human antibody sequences.
gacatccagatgacccagtctccatcctccctgtctgcatctgtaggagaccgtgtcaccatcacttgccgggcaagtcagagcattaggaagatgttagtttggtaccagcagaaaccagggaaagcccctaagctcctgatctatcgggcatcctatttgcaaagtggggtcccatcacgtttcagtggcagtggatctgggacagatttcactctcaccatcagcagtctgcaacctgaagattttgctacgtactactgtcaacaggcttttcggcggcctaggacgttcggccaagggaccaaggtggaaatcaaacgg

SEQ ID NO: 52 - protein, 108 aa, Artificial sequence
  Sequence selected from diversified human antibody sequences.
DIQMTQSPSSLSASVGDRVTITCRASQSIRKMLVWYQQKPGKAPKLLIYRASYLQSGVPSRFSGSGSGTDFTLTISSLQPEDFATYYCQQAFRRPRTFGQGTKVEIKR

SEQ ID NO: 53 - DNA, 345 bp, Artificial sequence
  Sequence selected from diversified human antibody sequences.
gaggtgcagctgttggagtctgggggaggcttggtacagcctggggggtccctgcgtctctcctgtgcagcctccggattcacctttgatctttataatatgttttgggtccgccaggctccagggaagggtctagagtgggtctcatttattagtcagactggtaggcttacatggtacgcagactccgtgaagggccggttcaccatctcccgcgacaattccaagaacacgctgtatctgcaaatgaacagcctgcgtgccgaggacaccgcggtatattactgtgcgaaaacgctggaggattttgactactggggccagggaaccctggtcaccgtctcg

SEQ ID NO: 54 - protein, 115 aa, Artificial sequence
  Sequence selected from diversified human antibody sequences.
EVQLLESGGGLVQPGGSLRLSCAASGFTFDLYNMFWVRQAPGKGLEWVSFISQTGRLTWYADSVKGRFTISRDNSKNTLYLQMNSLRAEDTAVYYCAKTLEDFDYWGQGTLVTVS

SEQ ID NO: 55 - DNA, 357 bp, Artificial sequence
  Sequence selected from diversified human antibody sequences.
gaggtgcagctgttggagtctgggggaggcttggtacagcctggggggtccctgcgtctctcctgtgcagcctccggattcacctttccggtttatatgatgggttgggtccgccaggctccagggaagggtctagagtgggtctcatcgattgatgctcttggtgggcggacaggttacgcagactccgtgaagggccggttcaccatctcccgcgacaattccaagaacacgctgtatctgcaaatgaacagcctgcgtgccgaggacaccgcggtatattactgtgcgaaaactatgtcgaataagacgcatacgtttgactactggggccagggaaccctggtcaccgtctcg

SEQ ID NO: 56 - protein, 119 aa, Artificial sequence
  Sequence selected from diversified human antibody sequences.
EVQLLESGGGLVQPGGSLRLSCAASGFTFPVYMMGWVRQAPGKGLEWVSSIDALGGRTGYADSVKGRFTISRDNSKNTLYLQMNSLRAEDTAVYYCAKTMSNKTHTFDYWGQGTLVTVS

SEQ ID NO: 57 - DNA, 345 bp, Artificial sequence
  Sequence selected from diversified human antibody sequences.
gaggtgcagctgttggagtctgggggaggcttggtacagcctggggggtccctgcgtctctcctgtgcagcctccggattcacctttgtggcttataatatgacttgggtccgccaggctccagggaagggtctagagtgggtctcaagtattaatacttttggtaattagacaaggtacgcagactccgtgaagggccggttcaccatctcccgcgacaattccaagaacacgctgtatctgcaaatgaacagcctgcgtgccgaggacaccgcggtatattactgtgcgaaaggtagtaggccttttgactactggggccagggaaccctggtcaccgtctcg

SEQ ID NO: 58 - protein, 114 aa, Artificial sequence
  Sequence selected from diversified human antibody sequences.
EVQLLESGGGLVQPGGSLRLSCAASGFTFVAYNMTWVRQAPGKGLEWVSSINTFGNTRYADSVKGRFTISRDNSKNTLYLQMNSLRAEDTAVYYCAKGSRPFDYWGQGTLVTVS

SEQ ID NO: 59 - DNA, 357 bp, Artificial sequence
  Sequence selected from diversified human antibody sequences.
gaggtgcagctgttggagtctgggggaggcttggtacagcctggggggtccctgcgtctctcctgtgcagcctccggattcaccttttaggggtatcgtatgggttgggtccgccaggctccagggaagggtctagagtgggtctcatggattacgcgtactggtgggacgacacagtacgcagactccgtgaagggccggttcaccatctcccgcgacaattccaagaacacgctgtatctgcaaatgaacagcctgcgtgccgaggacaccgcggtatattactgtgcgaaaccggcgaagcttgttggggttgggtttgactactggggccagggaaccctggtcaccgtctcg

SEQ ID NO: 60 - protein, 118 aa, Artificial sequence
  Sequence selected from diversified human antibody sequences.
EVQLLESGGGLVQPGGSLRLSCAASGFTFGYRMGWVRQAPGKGLEWVSWITRTGGTTQYADSVKGRFTISRDNSKNTLYLQMNSLRAEDTAVYYCAKPAKLVGVGFDYWGQGTLVTVS

SEQ ID NO: 61 - DNA, 357 bp, Artificial sequence
  Sequence selected from diversified human antibody sequences.
gaggtgcagctgttggagtctgggggaggcttggtacagcctggggggtccctgcgtctctcctgtgcagcctccggattcacctttcggaagtattagatggggtgggtccgccaggctccagggaagggtctagagtgggtctcacagattggtgcgaagggtcagtctacagattacgcagactccgtgaagggccggttcaccatctcccgcgacaattccaagaacacgctgtatctgcaaatgaacagcctgcgtgccgaggacaccgcggtatattactgtgcgaaaaagaagaggggggagaattatttttttgactactggggccagggaaccctggtcaccgtctcg

SEQ ID NO: 62 - protein, 118 aa, Artificial sequence
  Sequence selected from diversified human antibody sequences.
EVQLLESGGGLVQPGGSLRLSCAASGFTFRKYMGWVRQAPGKGLEWVSQIGAKGQSTDYADSVKGRFTISRDNSKNTLYLQMNSLRAEDTAVYYCAKKKRGENYFFDYWGQGTLVTVS

SEQ ID NO: 63 - DNA, 357 bp, Artificial sequence
  Sequence selected from diversified human antibody sequences.
gaggtgcagctgttggagtctgggggaggcttggtacagcctggggggtccctgcgtctctcctgtgcagcctccggattcacctttcggcggtatagtatgtcgtgggtccgccaggctccagggaagggtctagagtgggtctcagatatttctcgttctggtcggtatacacattacgcagactccgtgaagggccggttcaccatctcccgcgacaattccaagaacacgctgtatctgcaaatgaacagcctgcgtgccgaggacaccgcggtatattactgtgcgaaacgtattgattcttctcagaatgggtttgactactggggccagggaaccctggtcaccgtctcg

SEQ ID NO: 64 - protein, 119 aa, Artificial sequence
  Sequence selected from diversified human antibody sequences.
EVQLLESGGGLVQPGGSLRLSCAASGFTFRRYSMSWVRQAPGKGLEWVSDISRSGRYTHYADSVKGRFTISRDNSKNTLYLQMNSLRAEDTAVYYCAKRIDSSQNGFDYWGQGTLVTVS

SEQ ID NO: 65 - DNA, 345 bp, Artificial sequence
  Sequence selected from diversified human antiblody sequences.
gaggtgcagctgttggagtctgggggaggcttggtacagcctggggggtccctgcgtctctcctgtgcagcctccggattcaccttttaggggtataagatgttttgggtccgccaggctccagggaagggtctagagtgggtctcagctattagtggtagtggtggtagcacatactacgcagactccgtgaagggccggttcaccatctcccgcgacaattccaagaacacgctgtatctgcaaatgaacagcctgcgtgccgaggacaccgcggtatattactgtgcgaaacagaaggagaattttgactactggggccagggaaccctggtcaccgtctcg

SEQ ID NO: 66 - protein, 114 aa, Artificial sequence
  Sequence selected from diversified human antibody sequences.
EVQLLESGGGLVQPGGSLRLSCAASGFTFGYKMFWVRQAPGKGLEWVSAISGSGGSTYYADSVKGRFTISRDNSKNTLYLQMNSLRAEDTAVYYCAKQKENFDYWGQGTLVTVS

SEQ ID NO: 67 - DNA, 357 bp, Artificial sequence
  Sequence selected from diversified human antibody sequences.
gaggtgcagctgttggagtctgggggaggcttggtacagcctggggggtccctgcgtctctcctgtgcagcctccggattcacctttggggattatgctatgtggtgggtccgccaggctccagggaagggtctagagtgggtctcagtgattagttcgaatggtgggagtacattttacgcagactccgtgaagggccggttcaccatctcccgcgacaattccaagaacacgctgtatctgcaaatgaacagcctgcgtgccgaggacaccgcggtatattactgtgcgaaacgtgttcgtaagaggactcctgagtttgactactggggccagggaaccctggtcaccgtctcg

SEQ ID NO: 68 - protein, 119 aa, Artificial sequence
  Sequence selected from diversified human antibody sequences.
EVQLLESGGGLVQPGGSLRLSCAASGFTFGDYAMWWVRQAPGKGLEWVSVISSNGGSTFYADSVKGRFTISRDNSKNTLYLQMNSLRAEDTAVYYCAKRVRKRTPEFDYWGQGTLVTVS

SEQ ID NO: 69 - DNA, 357 bp, Artificial sequence
  Sequence selected from diversified human antibody sequences.
gaggtgcagctgttggagtctgggggaggcttggtacagcctggggggtccctgcgtctctcctgtgcagcctccggattcacctttaggaggtataagatgggttgggtccgccaggctccagggaagggtctagagtgggtctcagcgattgggaggaatggtacgaagacaaattacgcagactccgtgaagggccggttcaccatctcccgcgacaattccaagaacacgctgtatctgcaaatgaacagcctgcgtgccgaggacaccgcggtatattactgtgcgaaaatttatacggggaagcctgctgcgtttgactactggggccagggaaccctggtcaccgtctcg

SEQ ID NO: 70 - protein, 119 aa, Artificial sequence
  Sequence selected from diversified human antibody sequences.
EVQLLESGGGLVQPGGSLRLSCAASGFTFRRYKMGWVRQAPGKGLEWVSAIGRNGTKTNYADSVKGRFTISRDNSKNTLYLQMNSLRAEDTAVYYCAKIYTGKPAAFDYWGQGTLVTVS

SEQ ID NO: 71 - DNA, 357 bp, Artificial sequence
  Sequence selected from diversified human antibody sequences.
gaggtgcagctgttggagtctgggggaggcttggtacagcctggggggtccctgcgtctctcctgtgcagcctccggattcacctttaagaagtattagatgtcttgggtccgccaggctccagggaagggtctagagtgggtctcagctattagtggtagtggtggtagcacatactacgcagactccgtgaagggccggttcaccatctcccgcgacaattccaagaacacgctgtatctgcaaatgaacagcctgcgtgccgaggacaccgcggtatattactgtgcgaaaatgctgaggactaagaataaggtgtttgactactggggccagggaaccctggtcaccgtctcg

SEQ ID NO: 72 - protein, 118 aa, Artificial sequence
  Sequence selected from diversified human antibody sequences.
EVQLLESGGGLVQPGGSLRLSCAASGFTFKKYMSWVRQAPGKGLEWVSAISGSGGSTYYADSVKGRFTISRDNSKNTLYLQMNSLRAEDTAVYYCAKMLRTKNKVFDYWGQGTLVTVS

SEQ ID NO: 73 - DNA, 357 bp, Artificial sequence; identical to SEQ ID NO: 69
  Sequence selected from diversified human antibody sequences.

SEQ ID NO: 74 - protein, 119 aa, Artificial sequence; identical to SEQ ID NO: 70
  Sequence selected from diversified human antibody sequences.

SEQ ID NO: 75 - DNA, 357 bp, Artificial sequence
  Sequence selected from diversified human antibody sequences.
gaggtgcagctgttggagtctgggggaggcttggtacagcctggggggtccctgcgtctctcctgtgcagcctccggattcaccttttagagttatcggatgggttgggtccgccaggctccagggaagggtctagagtgggtctcaagtatttcgtcgaggggtaggcatacatcttacgcagactccgtgaagggccggttcaccatctcccgcgacaattccaagaacacgctgtatctgcaaatgaacagcctgcgtgccgaggacaccgcggtatattactgtgcgaaaagggttccgggtcgggggcgttcttttgactactggggccagggaaccctggtcaccgtctcg

SEQ ID NO: 76 - protein, 118 aa, Artificial sequence
  Sequence selected from diversified human antibody sequences.
EVQLLESGGGLVQPGGSLRLSCAASGFTFSYRMGWVRQAPGKGLEWVSSISSRGRHTSYADSVKGRFTISRDNSKNTLYLQMNSLRAEDTAVYYCAKRVPGRGRSFDYWGQGTLVTVS

SEQ ID NO: 77 - DNA, 357 bp, Artificial sequence
  Sequence selected from diversified human antibody sequences.
gaggtgcagctgttggagtctgggggaggcttggtacagcctggggggtccctgcgtctctcctgtgcagcctccggattcccctttcgtcggtatcggatgaggtgggtccgccaggctccagggaagggtctagagtgggtctcaggtatttctccgggtggtaagcatacaacgtacgcagactccgtgaagggccggttcaccatctcccgcgacaattccaagaacacgctgtatctgcaaatgaacagcctgcgtgccgaggacaccgcggtatattactgtgcgaaaggtgaggggggggcgagttctgcgtttgactactggggccagggaaccctggtcaccgtctcg

SEQ ID NO: 78 - protein, 119 aa, Artificial sequence
  Sequence selected from diversified human antibody sequences.
EVQLLESGGGLVQPGGSLRLSCAASGFPFRRYRMRWVRQAPGKGLEWVSGISPGGKHTTYADSVKGRFTISRDNSKNTLYLQMNSLRAEDTAVYYCAKGEGGASSAFDYWGQGTLVTVS

SEQ ID NO: 79 - DNA, 357 bp, Artificial sequence
  Sequence selected from diversified human antibody sequences.
gaggtgcagctgttggagtctgggggaggcttggtacagcctggggggtccctgcgtctctcctgtgcagcctccggattcaccttttagcggtatgggatggtttgggtccgccaggctccagggaagggtctagagtgggtctcagctattagtggtagtggtggtagcacatactacgcagactccgtgaagggccggttcaccatctcccgcgacaattccaagaacacgctgtatctgcaaatgaacagcctgcgtgccgaggacaccgcggtatattactgtgcgaaacggcatagttctgaggctaggcagtttgactactggggccagggaaccctggtcaccgtctcg

SEQ ID NO: 80 - protein, 118 aa, Artificial sequence
  Sequence selected from diversified human antibody sequences.
EVQLLESGGGLVQPGGSLRLSCAASGFTFRYGMVWVRQAPGKGLEWVSAISGSGGSTYYADSVKGRFTISRDNSKNTLYLQMNSLRAEDTAVYYCAKRHSSEARQFDYWGQGTLVTVS

SEQ ID NO: 81 - DNA, 381 bp, Artificial sequence
  Sequence selected from diversified human antibody sequences.
gcgtcgacggaggtccagctgttggagtctgggggaggcttggtacagcctggggggtccctgcgtctctcctgtgcagcctccggattcacctttgagtggtattggatgggttgggtccgccaggctccagggaagggtctagagtgggtctcagctatcagtggtagtggtggtagcacatactacgcagactccgtgaagggccggttcaccatctcccgcgacaattccaagaacacgctgtatctgcaaatgaacagcctgcgtgccgaggacgccgcggtatattactgtgcgaaagttaagttggggggggggcctaattttggctaccggggccagggaaccctggtcaccgtctcgtgctaataaggatcc

SEQ ID NO: 82 - protein, 123 aa, Artificial sequence
  Sequence selected from diversified human antibody sequences.
ASTEVQLLESGGGLVQPGGSLRLSCAASGFTFEWYWMGWVRQAPGKGLQWVSAISGSGGSTYYADSVKGRFTISRDNSKNTLYLQMNSLRAEDAAVYYCAKVKLGGGPNFGYRGQGTLVTVSC

SEQ ID NO: 83 - DNA, 351 bp, Artificial sequence
  Sequence selected from diversified human antibody sequences.
gaggtgcagctgttggagtctgggggaggcttagtacagcctggggggtccctgcgtctctcctgtgcagcctccggattcacctttattgcttatgatatgagttgggtccgccaggctccagggaagggtctggagtgggtctcatggattgatgagtggggtctgcagacatactacgcagactccgtgaagggccggttcaccatctcccgcgacaattccaagaacacgctgtatctgcaaatgaacagcctgcgtgccgaggacaccgcggtatattactgtgcgaaaaagacgcctgaggagtttgactactggggtcagggaaccctggtcaccgtctcgagc

SEQ ID NO: 84 - protein, 117 aa, Artificial sequence
  Sequence selected from diversified human antibody sequences.
EVQLLESGGGLVQPGGSLRLSCAASGFTFIAYDMSWVRQAPGKGLEWVSWIDEWGLQTYYADSVKGRFTISRDNSKNTLYLQMNSLRAEDTAVYYCAKKTPEEFDYWGQGTLVTVSS

SEQ ID NO: 85 - DNA, 324 bp, Artificial sequence
  Sequence selected from diversified human antibody sequences.
gacatccagatgacccagtctccatcctccctgtctgcatctgtaggagaccgtgtcaccatcacttgccgggcaagtcagcctattggtcctgatttactgtggtaccagcagaaaccagggaaagcccctaagctcctgatctatcagacgtccattttgcaaagtggggtcccatcacgtttcagtggcagtggatctgggacagatttcactctcaccatcagcagtctgcaacctgaagattttgctacgtactactgtcaacagtattgggcttttcctgtgacgttcggccaagggaccaaggtggaaatcaaacgg

SEQ ID NO: 86 - protein, 108 aa, Artificial sequence
  Sequence selected from diversified human antibody sequences.
DIQMTQSPSSLSASVGDRVTITCRASQPIGPDLLWYQQKPGKAPKLLIYQTSILQSGVPSRFSGSGSGTDFTLTISSLQPEDFATYYCQQYWAFPVTFGQGTKVEIKR

SEQ ID NO: 87 - protein, 120 aa, Artificial sequence
  Sequence selected from diversified human antibody sequences.
EVQLLESGGGLVQPGGSLRLSCAASGFPFEWYWMGWVRQAPGKGLEWVSAISGSGDSTYYADSVKGRFTISRDNSKNTLYQQMNSLRAEDAAVYYCAKVKLGGGPNFGYRGQGTLVTVSS

SEQ ID NO: 88 - protein, 14 aa, Artificial sequence
  C-Myc epitope tag appended to polypeptides expressed in pDOM5 vector.
AAAEQKLISEEDLN

SEQ ID NO: 89 - protein, 123 aa, Artificial sequence
  Sequence selected from diversified human antibody sequences.
EVQLLESGGGLVQPGGSLRLSCAASGFTFSKYWMSWVRQAPGKGLEWVSSIDFMGPHTYYADSVKGRFTISRDNSKNTLYLQMNSLRAEDTAVYYCAKGRTSMLPMKGKFDYWGQGTLVTVSS

SEQ ID NO: 90 - protein, 118 aa, Artificial sequence
  Sequence selected from diversified human antibody sequences.
EVQLLESGGGLVQPGGSLRLSCAASGFTFYDYNMSWVRQAPGKGLEWVSTITHTGGVTYYADSVKGRFTISRDNSKNTLYLQMNSLRAEDTAVYYCAKQNPSYQFDYWGQGTLVTVSS

SEQ ID NO: 91 - protein, 118 aa, Artificial sequence
  Sequence selected from diversified human antibody sequences.
EVQLLESGGGLVQPGGSLRLSCTASGFTFDEYNMSWVRQAPGKGLEWVSTILPHGDRTYYADSVKGRFTISRDNSKNTLYLQMNSLRAEDTAVYYCAKQDPLYRFDYWGQGTLVTVSS

SEQ ID NO: 92 - protein, 5 aa, Artificial sequence
  Consensus CDR1 sequence for MSA/HSA-specific dAbs.
XXXLX

SEQ ID NO: 93 - protein, 7 aa, Artificial sequence
  Consensus CDR2 sequence for MSA/HSA binders.
XASXLQS

SEQ ID NO: 94 - protein, 9 aa, Artificial sequence
  Consensus CDR3 sequence for MSA/HSA binders.
QQXXXXPXT

SEQ ID NO: 95 - protein, 5 aa, Artificial sequence
  Sequence selected from diversified human antibody sequences.
SSYLN

SEQ ID NO: 96 - protein, 7 aa, Artificial sequence
  Sequence selected from diversified human antibody sequences.
RASPLQS

SEQ ID NO: 97 - protein, 9 aa, Artificial sequence
  Sequence selected from diversified human antibody sequences.
QQTYSVPPT

SEQ ID NO: 98 - protein, 5 aa, Artificial sequence
  Sequence selected from diversified human antibody sequences.
SSYLN

SEQ ID NO: 99 - protein, 7 aa, Artificial sequence
  Sequence selected from diversified human antibody sequences.
RASPLQS

SEQ ID NO: 100 - protein, 9 aa, Artificial sequence
  Sequence selected from diversified human antibody sequences.
QQTYRIPPT

SEQ ID NO: 101 - protein, 5 aa, Artificial sequence
  Sequence selected from diversified human antibody sequences.
FKSLK

SEQ ID NO: 102 - protein, 7 aa, Artificial sequence
  Sequence selected from diversified human antibody sequences.
NASYLQS

SEQ ID NO: 103 - protein, 9 aa, Artificial sequence
  Sequence selected from diversified human antibody sequences.
QQVVYWPVT

SEQ ID NO: 104 - protein, 5 aa, Artificial sequence
  Sequence selected from diversified human antibody sequences.
YYHLK

SEQ ID NO: 105 - protein, 7 aa, Artificial sequence
  Sequence selected from diversified human antibody sequences.
KASTLQS

SEQ ID NO: 106 - protein, 9 aa, Artificial sequence
  Sequence selected from diversified human antibody sequences.
QQVRKVPRT

SEQ ID NO: 107 - protein, 5 aa, Artificial sequence
  Sequence selected from diversified human antibody sequences.
RRYLK

SEQ ID NO: 108 - protein, 7 aa, Artificial sequence
  Sequence selected from diversified human antibody sequences.
QASVLQS

SEQ ID NO: 109 - protein, 9 aa, Artificial sequence
  Sequence selected from diversified human antibody sequences.
QQGLYPPIT

SEQ ID NO: 110 - protein, 5 aa, Artificial sequence
  Sequence selected from diversified human antobody sequences.
YNWLK

SEQ ID NO: 111 - protein, 7 aa, Artificial sequence
  Sequence selected from diversified human antibody sequences.
RASSLQS

SEQ ID NO: 112 - protein, 9 aa, Artificial sequence
  Sequence selected from diversified human antibody sequences.
QQNVVIPRT

SEQ ID NO: 113 - protein, 5 aa, Artificial sequence
  Sequence selected from diversified human antibody sequences.
LWHLR

SEQ ID NO: 114 - protein, 7 aa, Artificial sequence
  Sequence selected from diversified human antibody sequences.
HASLLQS

SEQ ID NO: 115 - protein, 9 aa, Artificial sequence
  Sequence selected from diversified human antibody sequences.
QQSAVYPKT

SEQ ID NO: 116 - protein, 5 aa, Artificial sequence
  Sequence selected from diversified human antibody sequences.
FRYLA

SEQ ID NO: 117 - protein, 7 aa, Artificial sequence
  Sequence selected from diversified human antibody sequences.
HASHLQS

SEQ ID NO: 118 - protein, 9 aa, Artificial sequence
  Sequence selected from diversified human antibody sequences.
QQRLLYPKT

SEQ ID NO: 119 - protein, 5 aa, Artificial sequence
  Sequence selected from diversified human antibody sequences.
FYHLA

SEQ ID NO: 120 - protein, 7 aa, Artificial sequence
  Sequence selected from diversified human antibody sequences.
PASKLQS

SEQ ID NO: 121 - protein, 9 aa, Artificial sequence
  Sequence selected from diversified human antibody sequences.
QQRARWPRT

SEQ ID NO: 122 - protein, 5 aa, Artificial sequence
  Sequence selected from diversified human antibody sequences.
IWHLN

SEQ ID NO: 123 - protein, 7 aa, Artificial sequence
  Sequence selected from diversified human antibody sequences.
RASRLQS

SEQ ID NO: 124 - protein, 9 aa, Artificial sequence
  Sequence selected from diversified human antibody sequences.
QQVARVPRT

SEQ ID NO: 125 - protein, 5 aa, Artificial sequence
  Sequence selected from diversified human antibody sequences.
YRYLR

SEQ ID NO: 126 - protein, 7 aa, Artificial sequence
  Sequence selected from diversified human antibody sequences.
KASSLQS

SEQ ID NO: 127 - protein, 9 aa, Artificial sequence
  Sequence selected from diversified human antibody sequences.
QQYVGYPRT

SEQ ID NO: 128 - protein, 5 aa, Artificial sequence
  Sequence selected from diversified human antibody sequences.
LKYLK

SEQ ID NO: 129 - protein, 7 aa, Artificial sequence
  Sequence selected from diversified human antibody sequences.
NASHLQS

SEQ ID NO: 130 - protein, 9 aa, Artificial sequence
  Sequence selected from diversified human antibody sequences.
QQTTYYPIT

SEQ ID NO: 131 - protein, 5 aa, Artificial sequence
  Sequence selected from diversified human antibody sequences.
LRYLR

SEQ ID NO: 132 - protein, 7 aa, Artificial sequence
  Sequence selected from diversified human antibody sequences.
KASWLQS

SEQ ID NO: 133 - protein, 9 aa, Artificial sequence
  Sequence selected from diversified human antibody sequences.
QQVLYYPQT

SEQ ID NO: 134 - protein, 5 aa, Artificial sequence
  Sequence selected from diversified human antibody sequences.
LRSLK

SEQ ID NO: 135 - protein, 7 aa, Artificial sequence
  Sequence selected from diversified human antibody sequences.
AASRLQS

SEQ ID NO: 136 - protein, 9 aa, Artificial sequence
  Sequence selected from diversified human antibody sequences.
QQVVYWPAT

SEQ ID NO: 137 - protein, 5 aa, Artificial sequence
  Sequence selected from diversified human antibody sequences.
FRHLK

SEQ ID NO: 138 - protein, 7 aa, Artificial sequence
  Sequence selected from diversified human antibody sequences.
AASRLQS

SEQ ID NO: 139 - protein, 9 aa, Artificial sequence
  Sequence selected from diversified human antibody sequence.
QQVALYPKT

SEQ ID NO: 140 - protein, 5 aa, Artificial sequence
  Sequence selected from diversified human antibody sequences.
RKYLR

SEQ ID NO: 141 - protein, 7 aa, Artificial sequence
  Sequence selected from diversified human antibody sequences.
TASSLQS

SEQ ID NO: 142 - protein, 9 aa, Artificial sequence
  Sequence selected from diversified human antibody sequences.
QQNLFWPRT

SEQ ID NO: 143 - protein, 5 aa, Artificial sequence
  Sequence selected from diversified human antibody sequences.
RRYLN

SEQ ID NO: 144 - protein, 7 aa, Artificial sequence
  Sequence selected from diversified human antibody sequences.
AASSLQS

SEQ ID NO: 145 - protein, 9 aa, Artificial sequence
  Sequence selected from diversified human antibody sequences.
QQMLFYPKT

SEQ ID NO: 146 - protein, 5 aa, Artificial sequence
  Sequence selected from diversified human antibody sequences.
IKHLK

SEQ ID NO: 147 - protein, 7 aa, Artificial sequence
  Sequence selected from diversified human antibody sequences.
GASRLQS

SEQ ID NO: 148 - protein, 9 aa, Artificial sequence
  Sequence selected from diversified human antibody sequences.
QQGARWPQT

SEQ ID NO: 149 - protein, 5 aa, Artificial sequence
  Sequence selected from diversified human antibody sequences.
YYHLK

SEQ ID NO: 150 - protein, 7 aa, Artificial sequence
  Sequence selected from diversified human antibody sequences.
KASTLQS

SEQ ID NO: 151 - protein, 9 aa, Artificial sequence
  Sequence selected from diversified human antibody sequences.
QQVRKVPRT

SEQ ID NO: 152 - protein, 5 aa, Artificial sequence
  Sequence selected from diversified human antibody sequences.
YKHLK

SEQ ID NO: 153 - protein, 7 aa, Artificial sequence
  Sequence selected from diversified human antibody sequences.
NASHLQS

SEQ ID NO: 154 - protein, 9 aa, Artificial sequence
  Sequence selected from diversified human antibody sequences.
QQVGRYPKT

SEQ ID NO: 155 - protein, 5 aa, Artificial sequence
  Sequence selected from diversified human antibody sequences.
FKSLK

SEQ ID NO: 156 - protein, 7 aa, Artificial sequence
  Sequence selected from diversified human antibody sequences.
NASYLQS

SEQ ID NO: 157 - protein, 9 aa, Artificial sequence
  Sequence selected from diversified human antibody sequences.
QQVVYWPVT

SEQ ID NO: 158 - protein, 6 aa, Artificial sequence
  Consensus VH CDR1 sequence.
XXYXXX

SEQ ID NO: 159 - protein, 17 aa, Artificial sequence
  Consensus VH CDR2 sequence for MSA/HSA binders.
XIXXXGXXTXYADSVKG

SEQ ID NO: 160 - protein, 11 aa, Artificial sequence
  Consensus sequence for VH CDR3 of MSA/HSA binders.
XXXXXXXXFDY

SEQ ID NO: 161 - protein, 6 aa, Artificial sequence
  Sequence selected from diversified human antibody sequences.
WVYQMD

SEQ ID NO: 162 - protein, 17 aa, Artificial sequence
  Sequence selected from diversified human antibody sequences.
SISAFGAKTLYADSVKG

SEQ ID NO: 163 - protein, 7 aa, Artificial sequence
  Sequence selected from diversified human antibody sequences.
LSGKFDY

SEQ ID NO: 164 - protein, 6 aa, Artificial sequence
  Sequence selected from diversified human antibody sequences.
WSYQMT

SEQ ID NO: 165 - protein, 18 aa, Artificial sequence
  Sequence selected from diversified human antibody sequences.
SISSFGSSTYLYADSVKG

SEQ ID NO: 166 - protein, 11 aa, Artificial sequence
  Sequence selected from diversified human antibody sequences.
GRDHNYSLFDY

SEQ ID NO: 167 - DNA, 27 bp, Artificial sequence
  Sequence encoding influenza hemaglutinin epitope tag.
tatccttatgatgttcctgattatgca

SEQ ID NO: 168 - protein, 9 aa, Artificial sequence
  Amino acid sequence of the influenza hemaglutinin epitope tag.
YPYDVPDYA

SEQ ID NO: 169 - DNA, 39 bp, Artificial sequence
  Forward oligonucleotide for the amplification and introduction of cloning sites onto the TAR1-5-19 sequence.
tggagcgcgtcgacggacatccagatgacccagtctcca

SEQ ID NO: 170 - DNA, 39 bp, Artificial sequence
  Reverse oligonucleotide for the amplification and introduction of cloning sites onto the TAR1-5-19 sequence.
ttagcagccggatccttattagcaccgtttgatttccac

SEQ ID NO: 171 - DNA, 357 bp, Artificial sequence
  Sequence encoding anti-TNF-alpha binding dAb TAR1-5-19 plus restriction sites for cloning and introduction of a C-terminal Cys residue.
tggagcgcgtcgacggacatccagatgacccagtctccatcctctctgtctgcatctgtaggagaccgtgtcaccatcacttgccgggcaagtcagagcattgatagttatttacattggtaccagcagaaaccagggaaagcccctaagctcctgatctatagtgcatccgagttgcaaagtggggtcccatcacgtttcagtggcagtggatctgggacagatttcactctcaccatcagcagtctgcaacctgaagattttgctacgtactactgtcaacaggttgtgtggcgtccttttacgttcggccaagggaccaaggtggaaatcaaacggtgctaataaggatccggc

SEQ ID NO: 172 - protein, 114 aa, Artificial sequence
  Amino acid sequence of TAR1-5-19 anti-TNF-alpha dAb encoded by nucleic acid construct of the immediately preceding SEQ ID.
WSASTDIQMTQSPSSLSASVGDRVTITCRASQSVKEFLWWYQQKPGKAPKLLIYMASNLQSGVPSRFSGSGSGTDFTLTISSLQPEDFATYYCQQKFKLPRTFGQGTKVEIKRC

SEQ ID NO: 173 - DNA, 22 bp, Artificial sequence
  Forward PCR oligo used to introduce cloning sites onto Vk sequence used for library preparation.
cggccatggcgtcaacggacat

SEQ ID NO: 174 - DNA, 23 bp, Artificial sequence
  Reverse PCR oligo used to introduce cloning site onto V kappa sequence used for library preparation.
atgtgcgctcgagcgtttgattt

SEQ ID NO: 175 - protein, 15 aa, Artificial sequence
  Modified form of the human IgGC1 hinge appended to C terminus of a dAb.
EPKSGDKTHTCPPCP

(19) United States
(12) Patent Application Publication
(10) Pub. No.: US 2009/0148434 A1
(43) Pub. Date: Jun. 11, 2009

(54) Antibody Compositions and Methods

(75) Inventors: Ian Tomlinson, Cambridge (GB); Amrik Basran, Cambridge (GB); Philip Jones, Cambridge (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: Domantis Limted

(21) Appl. No.: 12/186,281

(22) Filed: Aug. 5, 2008

Related U.S. Application Data
(62) Division of application No. 11/401,745, filed on Apr. 10, 2006.
Application No. 11/401,745 is a continuation of application No. PCT/GB2004/004253, filed on Oct. 8, 2004.
(60) Provisional application No. 60/535,076, filed on Jan. 8, 2004.
(60) Provisional application No. 60/509,613, filed on Oct. 8, 2003.

(30) Foreign Application Priority Data
Jun. 30, 2004 (GB) PCT/GB2004/002829

(51) Int. Cl. A61K 39/395; C07K 16/18

(52) U.S. Cl. 424/130.1; 530/387.1; 424/158.1

(57) ABSTRACT

Provided are concentrated preparations comprising single immunoglobulin variable domain polypeptides that bind target antigen with high affinity and are soluble at high concentration, without aggregation or precipitation, providing, for example, for increased storage stability and the ability to administer higher therapeutic doses.

This application is a continuation of PCT/GB2004/004253, filed Oct. 8, 2004, which claims priority to PCT/GB2004/002829, filed Jun. 30, 2004, U.S. provisional application No. 60/535,076, filed Jan. 8, 2004, and U.S. provisional application No. 60/509,613, filed Oct. 8, 2003. The disclosure of each of these priority applications is hereby incorporated by reference herein in its entirety.

### BACKGROUND OF THE INVENTION

Conventional antibodies are large multi-subunit protein molecules comprising at least four polypeptide chains. For example, human IgG has two heavy chains and two light chains that are disulfide bonded to form the functional antibody. The size of a conventional IgG is about 150 kD. Because of their relatively large size, complete antibodies (e.g., IgG, IgA, IgM, etc.) are limited in their therapeutic usefulness due to problems in, for example, tissue penetration. Considerable efforts have focused on identifying and producing smaller antibody fragments that retain antigen binding function and solubility.

The heavy and light polypeptide chains of antibodies comprise variable (V) regions that directly participate in antigen interactions, and constant (C) regions that provide structural support and function in non-antigen-specific interactions with immune effectors. The antigen binding domain of a conventional antibody is comprised of two separate domains: a heavy chain variable domain (V_{H}) and a light chain variable domain (V_{L}: which can be either V_{κ} or V_{λ}). The antigen binding site itself is formed by six polypeptide loops: three from the V_{H} domain (H1, H2 and H3) and three from the V_{L} domain (L1, L2 and L3). In vivo, a diverse primary repertoire of V genes that encode the V_{H} and V_{L} domains is produced by the combinatorial rearrangement of gene segments. C regions include the light chain C regions (referred to as C_{L} regions) and the heavy chain C regions (referred to as C_{H}1, C_{H}2 and C_{H}3 regions).

A number of smaller antigen binding fragments of naturally occurring antibodies have been identified following protease digestion. These include, for example, the “Fab fragment” (V_{L}-C_{L}-C_{H}1-V_{H}), “Fab′ fragment” (a Fab with the heavy chain hinge region) and “F(ab′)₂ fragment” (a dimer of Fab′ fragments joined by the heavy chain hinge region). Recombinant methods have been used to generate even smaller antigen-binding fragments, referred to as “single chain Fv” (variable fragment) or “scFv,” consisting of V_{L} and V_{H} joined by a synthetic peptide linker.

While the antigen binding unit of a naturally-occurring antibody (e.g., in humans and most other mammals) is generally known to be comprised of a pair of V regions (V_{L}/V_{H}), camelid species express a large proportion of fully functional, highly specific antibodies that are devoid of light chain sequences. The camelid heavy chain antibodies are found as homodimers of a single heavy chain, dimerized via their constant regions. The variable domains of these camelid heavy chain antibodies are referred to as V_{H}H domains and retain the ability, when isolated as fragments of the V_{H} chain, to bind antigen with high specificity ((Hamers-Casterman et al., 1993, Nature 363: 446-448; Gahroudi et al., 1997, FEBS Lett. 414: 521-526). Antigen binding single V_{H} domains have also been identified from, for example, a library of murine V_{H} genes amplified from genomic DNA from the spleens of immunized mice and expressed in E. coli (Ward et al., 1989, Nature 341: 544-546). Ward et al. named the isolated single V_{H} domains “dAbs,” for “domain antibodies.” The term “dAb” will refer herein to a single immunoglobulin variable domain (V_{H} or V_{L}) polypeptide that specifically binds antigen. A “dAb” binds antigen independently of other V domains; however, as the term is used herein, a “dAb” can be present in a homo- or heteromultimer with other V_{H} or V_{L} domains where the other domains are not required for antigen binding by the dAb, i.e., where the dAb binds antigen independently of the additional V_{H} or V_{L} domains.

Single immunoglobulin variable domains, for example, V_{H}H, are the smallest antigen-binding antibody unit known. For use in therapy, human antibodies are preferred, primarily because they are not as likely to provoke an immune response when administered to a patient. As noted above, isolated non-camelid V_{H} domains tend to be relatively insoluble and are often poorly expressed. Comparisons of camelid V_{H}H with the V_{H} domains of human antibodies reveals several key differences in the framework regions of the camelid V_{H}H domain corresponding to the V_{H}/V_{L} interface of the human V_{H} domains. Mutation of these residues of human V_{H}3 to more closely resemble the V_{H}H sequence (specifically Gly 44→Glu, Leu 45→Arg and Trp 47→Gly) has been performed to produce “camelized” human V_{H} domains that retain antigen binding activity (Davies & Riechmann, 1994, FEBS Lett. 339: 285-290) yet have improved expression and solubility. (Variable domain amino acid numbering used herein is consistent with the Kabat numbering convention (Kabat et al., 1991, Sequences of Immunological Interest, 5^{th} ed. U.S. Dept. Health & Human Services, Washington, D.C.)) WO 03/035694 (Muyldermans) reports that the Trp 103→Arg mutation improves the solubility of non-camelid V_{H} domains. Davies & Riechmann (1995, Biotechnology N.Y. 13: 475-479) also report production of a phage-displayed repertoire of camelized human V_{H} domains and selection of clones that bind hapten with affinities in the range of 100-400 nM, but clones selected for binding to protein antigen had weaker affinities.

WO 00/29004 (Plaskin et al.) and Reiter et al. (1999, J. Mol. Biol. 290: 685-698) describe isolated V_{H} domains of mouse antibodies expressed in E. coli that are very stable and bind protein antigens with affinity in the nanomolar range. WO 90/05144 (Winter et al.) describes a mouse V_{H} domain antibody fragment that binds the experimental antigen lysozyme with a dissociation constant of 19 nM.

WO 02/051870 (Entwistle et al.) describes human V_{H} single domain antibody fragments that bind experimental antigens, including a V_{H} domain that binds an scFv specific for a Brucella antigen with an affinity of 117 nM, and a V_{H} domain that binds an anti-FLAG IgG.

Tanha et al. (2001, J. Biol. Chem. 276: 24774-24780) describe the selection of camelized human V_{H} domains that bind two monoclonal antibodies used as experimental antigens and have dissociation constants in the micromolar range.

U.S. Pat. No. 6,090,382 (Salfeld et al.) describe human antibodies that bind human TNF-α with affinities of 10⁻⁸ M or less, have an off-rate (K_{off}) for dissociation of human TNF-α of 10⁻³ sec⁻¹ or less and neutralize human TNF-α activity in a standard L929 cell assay.

### SUMMARY OF THE INVENTION

The invention provides concentrated preparations comprising human single immunoglobulin variable domain polypeptides that bind target antigen with high affinity. The variable domain polypeptides of the subject preparations are significantly smaller than conventional antibodies and the V domain monomers are smaller even than scFv molecules, which can improve in vivo target access when applied to therapeutic approaches. The relatively small size and high binding affinity of these polypeptides also permits them to bind more target per unit mass than preparations of larger antibody molecules, permitting lower doses with improved efficacy.

The human single immunoglobulin variable domain polypeptides disclosed herein can be highly concentrated without the aggregation or precipitation often seen with non-camelid single domain antibodies, providing, for example, for relative ease in expression, increased storage stability and the ability to administer higher therapeutic doses. The relatively small size of human single immunoglobulin variable domain polypeptides described herein also provides flexibility with respect to the format of the binding polypeptide for particular uses. For example, due to their small size, the human single immunoglobulin variable domain polypeptides described herein can be fused or linked to, e.g., effectors, targeting molecules, or agents that increase biological half-life, while still resulting in a molecule of smaller size relative to similar arrangements made using conventional antibodies. Also encompassed are multimers of the subject polypeptides, such as homodimers and homotrimers, which exhibit increased avidity over monomeric forms, and heteromultimers which have additional functional properties conferred by their heteromeric component(s).

In one aspect, the invention encompasses a composition comprising a polypeptide comprising a single human immunoglobulin variable domain that binds a polypeptide antigen with a K_{d} of less than or equal to 100 nM, wherein the polypeptide is present at a concentration of at least 400 μM as determined by absorbance of light at 280 nm wavelength.

In one embodiment, the polypeptide is present at a concentration of 400 μM to 20 mM.

In another embodiment, the polypeptide antigen is a human polypeptide antigen.

In another embodiment, the single human immunoglobulin variable domain is a V_{H} domain.

In another embodiment, the polypeptide consists of a human immunoglobulin V domain.

In another embodiment, the immunoglobulin V domain is of non-human mammalian origin, and is, for example, a non-human mammalian V_{L} domain. Non-human mammals from which V_{L} domains can be derived include, as non-limiting examples, mouse, rat, cow, pig, goat, horse, monkey, etc.

In another aspect, the invention encompasses a composition comprising a polypeptide comprising a single immunoglobulin V_{H} domain that binds a polypeptide antigen with a K_{d} of less than or equal to 100 nM, wherein the residue at position 103 (per Kabat numbering) is an arginine, and wherein the polypeptide is present at a concentration of at least 400 μM as determined by absorbance of light at 280 nm wavelength. The V_{H} domain according to this aspect can be human or non-human, e.g., a camelid V_{H}H or other non-human species, e.g., mouse, rat, cow, pig, goat, horse, monkey, etc. In one embodiment, the polypeptide is present at a concentration of 400 μM to 20 mM. In another embodiment, the polypeptide antigen is a human polypeptide antigen.

In another embodiment, the amino acid residue at position 45 is a non-charged amino acid. In another embodiment, the amino acid at position 45 is a leucine.

In another embodiment, the amino acid residue at position 44 is a glycine.

In another embodiment, the amino acid residue at position 47 is a non-charged amino acid. In another embodiment, the amino acid residue at position 47 is a tryptophan.

In another embodiment, the amino acid residue at position 44 is a glycine and the amino acid residue at position 45 is a leucine.

In another embodiment, the amino acid residue at position 44 is a glycine and the amino acid residue at position 47 is a tryptophan.

In another embodiment, the amino acid residue at position 45 is a leucine and the amino acid residue at position 47 is a tryptophan.

In another embodiment, the amino acid residue at position 44 is a glycine, the amino acid residue at position 45 is a leucine and the amino acid residue at position 47 is a tryptophan.

In another embodiment, the single immunoglobulin variable domain comprises a universal framework. In another embodiment, the universal framework comprises a V_{H} framework selected from the group consisting of those encoded by human germline gene segments DP47, DP45 and DP38 or the V_{L} framework encoded by human germline gene segment DPK9.

In another embodiment, one or more framework (FW) regions of the immunoglobulin variable domain comprise (a) the amino acid sequence of a human framework region, (b) at least 8 contiguous amino acids of the amino acid sequence of a human framework region, or (c) an amino acid sequence encoded by a human germline antibody gene segment, wherein the framework regions are as defined by Kabat. For example, in one embodiment, the immunoglobulin variable domain comprises a FW2 region encoded by a human germline antibody gene segment.

In another embodiment, the amino acid sequence of one or more of the framework regions is the same as the amino acid sequence of a corresponding framework region encoded by a human germline antibody gene segment, or the amino acid sequences of one or more of the framework regions collectively comprise up to 5 amino acid differences relative to the amino acid sequence of the corresponding framework region encoded by a human germline antibody gene segment.

In another embodiment, the amino acid sequences of framework regions FW1, FW2, FW3 and FW4 are the same as the amino acid sequence of corresponding framework regions encoded by a human germline antibody gene segment, or the amino acid sequences of FW1, FW2, FW3 and FW4 collectively contain up to 1, 2, 3, 4, 5, 6, 7, 8, 9 or 10 amino acid differences relative to the sequences of corresponding framework regions encoded by the human germline antibody gene segment.

In another embodiment, the single human immunoglobulin variable domain is a V_{H} domain having the sequence encoded by germline V_{H} gene segment DP47 but which differs in sequence from that encoded by DP47 at one or more positions selected from the group consisting of H30, H31, H33, H35, H50, H52, H52a, H53, H55, H56, H58, H95, H97 and H98.

In another embodiment, the V_{H} domain comprises the sequence encoded by germline V_{H} gene segment DP47 but which differs in sequence from that encoded by DP47 at one or more positions selected from the group consisting of H30, H31, H32, H33, H35, H50, H52, H52a, H53, H54, H55, H56, H58, H94, H95, H96, H97, H08, H99, H100, H100a, H100b, H100c, H100d, H100e, H100f, H100g, H101, and H102.

In another embodiment, the V_{H} domain comprises the sequence encoded by germline V_{H} gene segment DP47 but which differs in sequence from that encoded by DP47 at one or more positions selected from the group consisting of H30, H31, H33, H35, H50, H52, H52a, H53, H55, H56, H58, H95, H96, H97, H08, H99, H100, H100a, H100b, H100c, H100d, H100e, and H100f.

In another embodiment, the single human immunoglobulin variable domain is a V_{H} domain having the sequence encoded by germline V_{H} gene segment DP47 but which differs in sequence from that encoded by DP47 at one or more positions selected from the group consisting of H30, H31, H33, H35, H50, H52, H52a, H53, H55, H56, H58, H95, H97, H98, H99, H100, H100a and H100b.

In another embodiment, the single human immunoglobulin variable domain is a V_{L} domain. In another embodiment, the polypeptide consists of a single human immunoglobulin V_{L} domain.

In another embodiment, the V_{L} domain is a V_{κ} domain.

In another embodiment, the V_{κ} domain comprises the sequence encoded by germline V_{κ} gene segment DPK9 but which differs in sequence from that encoded by DPK9 at one or more positions selected from the group consisting of L30, L31, L32, L34, L50, L53, L91, L92, L93, L94 and L96.

In another embodiment, the V_{κ} domain comprises the sequence encoded by germline V_{κ} gene segment DPK9 but which differs in sequence from that encoded by DPK9 at one or more positions selected from the group consisting of L28, L30, L31, L32, L34, L50, L51, L53, L91, L92, L93, L94, and L96.

In another embodiment, the composition further comprises a pharmaceutically acceptable carrier.

In another embodiment, the polypeptide binds the target antigen with a K_{d} of 100 nM to 50 pM.

In another embodiment, the polypeptide binds the antigen with a K_{d} of 30 nM to 50 pM.

In another embodiment, the polypeptide binds the target antigen with a K_{d} of 10 nM to 50 pM.

In any of the embodiments described herein, the antigen can be selected from, for example, the group including or consisting of human cytokines, cytokine receptors, enzymes, co-factors for enzymes and DNA binding proteins. In any of the embodiments described herein, preferred target antigens for the single domain immunoglobulin polypeptides include, but are not limited to, for example, TNF-α, p55 TNFR, EGFR, matrix metalloproteinase (MMP)-12, IgE, serum albumin, interferon γ, CEA and PDK1. Amino acid sequences for these target antigens are known to those of skill in the art. Given the amino acid sequence of the antigen, one of skill in the art can generate antigen for use in selecting immunoglobulin polypeptides that specifically bind the antigen. As examples, the sequence of human MMP-12 is described by Shariro et al., 1993, J. Biol. Chem. 268: 23824-23829 and in GenBank Accession No. P39900; the sequence of human TNF-α is reported by Shirai et al., 1985, Nature 313: 803-806 and in GenBank Accession No. P01375; the sequence of human p55 TNFR is described by Loetscher et al., 1990, Cell 61: 351-359 and in GenBank Accession No. P19438; the sequence of human serum albumin is at GenBank Accession No. AAU21642; a human IgE sequence is available at GenBank Accession No. CAA65057; the sequence of human interferon γ is at GenBank Accession No. CAA00226; the sequence of human carcinoembryonic antigen is at GenBank Accession No. AAA51971; and the sequence of human PDK1 is at GenBank Accession No. O15530. There are often also commercial sources for antigen polypeptides. It is further preferred, although not required, that these and other antigens be human antigens.

In another embodiment, the antigen is human TNF-α. In another embodiment, the polypeptide neutralizes human TNF-α in a standard L929 in vitro assay, with an IC₅₀ of 100 nM or less.

In another embodiment, the polypeptide comprises the sequence of TAR1-5-19 (SEQ ID NO: 16) or a sequence at least 90% similar to SEQ ID NO: 16.

In another embodiment, the antigen is human TNF-α receptor p55. In another embodiment, the polypeptide inhibits the cytotoxic effect of human TNF-α in a standard L929 in vitro assay, with an IC₅₀ of 100 nM or less.

In another embodiment, the polypeptide comprises the sequence of TAR2 (SEQ ID NO: 14) or a sequence at least 90% similar to SEQ ID NO: 14.

In another embodiment of each aspect of the concentrated single immunoglobulin variable domain compositions described herein, the single immunoglobulin variable domain polypeptide comprises a sequence selected from the group consisting of SEQ ID NO: 2, 4, 6, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 87, 89, 90 and 91.

The invention further encompasses a method of preparing a composition comprising a single human immunoglobulin variable domain polypeptide that binds a polypeptide antigen with a K_{d} of less than or equal to 100 nM, wherein the polypeptide is present at a concentration of at least 400 μM as determined by absorbance of light at 280 nm wavelength, the method comprising the steps of expressing a nucleic acid encoding a single immunoglobulin variable domain polypeptide in a host cell, wherein the polypeptide binds a polypeptide antigen with a kD of less than or equal to 100 nM, and concentrating the single immunoglobulin variable domain polypeptide to a concentration of at least 400 μM as determined by absorbance at A280.

In one embodiment, the nucleic acid comprises the sequence of one of SEQ ID NOs 1, 3, 5, 13, 15, 17, 19, 21, 23, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83 and 85 or a sequence at least 90% identical to one of these. Another embodiment encompasses a vector comprising such a nucleic acid.

The invention further encompasses a homomultimer of a single human immunoglobulin variable domain polypeptide that binds a human antigen with a K_{d} of less than or equal to 100 nM, wherein the polypeptide is present at a concentration of at least 400 μM.

In one embodiment, the homomultimer is a homodimer or a homotrimer.

In another embodiment, one or more monomers comprised by the homomultimer are linked via a free C terminal cysteine residue. In another embodiment, the monomers further comprise a linker peptide sequence, and the free cysteine residue is located at the C terminus of the linker peptide sequence. In another embodiment, monomers in such a homodimer are linked via disulfide bonds.

In another embodiment, the homomultimer is a homotrimer and the monomers in the homotrimer are chemically linked by thiol linkages with TMEA.

In another embodiment, the monomers of the homomultimer are specific for a multi-subunit target. In another embodiment, the target is human TNF-α.

The invention further encompasses a heteromultimer of a single immunoglobulin variable domain polypeptide that binds a polypeptide antigen with a K_{d} of less than or equal to 100 nM, wherein the polypeptide is present at a concentration of at least 400 μM. In one embodiment, the heteromultimer is a heterodimer or heterotrimer. In another embodiment, the single immunoglobulin variable domain polypeptide is a human single immunoglobulin variable domain polypeptide. In another embodiment, the polypeptide antigen is a human polypeptide antigen.

The invention further encompasses a composition comprising an extended release formulation comprising a single immunoglobulin variable domain. In one embodiment, the single immunoglobulin variable domain is a non-human mammalian single immunoglobulin variable domain, e.g., a camelid or other non-human species single immunoglobulin variable domain. In another embodiment, the single immunoglobulin variable domain is a human single immunoglobulin variable domain.

The invention further encompasses a method of treating or preventing a disease or disorder in an individual in need of such treatment, the method comprising administering to the individual a therapeutically effective amount of a composition comprising a polypeptide comprising a single human immunoglobulin variable domain that binds a polypeptide antigen with a K_{d} of less than or equal to 100 nM, wherein the polypeptide is present at a concentration of at least 400 μM.

In one embodiment, the single human immunoglobulin variable domain specifically binds a human polypeptide antigen. In another embodiment, the single human immunoglobulin variable domain specifically binds TNF-α or TNF-α p55 receptor.

The invention further encompasses a method of increasing the in vivo half-life of a composition comprising a polypeptide comprising a single human immunoglobulin variable domain that binds a polypeptide antigen with a K_{d} of less than or equal to 100 nM, wherein the polypeptide is present at a concentration of at least 400 μM, the method comprising covalently linking a polymer molecule to the composition.

In one embodiment, the polymer comprises a substituted or unsubstituted straight or branched chain polyalkylene, polyalkenylene or polyoxyalkylene polymer or a branched or unbranched polysaccharide.

In another embodiment, the polymer comprises a substituted or unsubstituted straight or branched chain polyethylene glycol or polyvinyl alcohol.

In another embodiment, the polymer comprises methoxy(polyethylene glycol).

In another embodiment, the polymer comprises polyethylene glycol. In another embodiment, the molecular weight of the polyethylene glycol is 5,000 to 50,000 kD.

The invention further encompasses a method of increasing the half-life of a single immunoglobulin variable domain polypeptide composition, the method comprising linking the single immunoglobulin variable domain to a second single immunoglobulin variable domain polypeptide that binds a polypeptide that increases the serum half-life of the construct. In one embodiment, the second single immunoglobulin variable domain polypeptide binds a serum albumin, e.g., human serum albumin.

The invention further encompasses a composition comprising a polypeptide comprising a single immunoglobulin variable domain that binds a polypeptide antigen with a K_{d} of less than or equal to 100 nM, wherein the polypeptide is present at a concentration of at least 400 μM, and wherein the polypeptide is further linked to a second single immunoglobulin variable domain polypeptide that binds a molecule that increases the half-life of the construct. In one embodiment, the second single immunoglobulin variable domain polypeptide binds a serum albumin, e.g., human serum albumin.

The invention further encompasses a composition comprising a polypeptide comprising a single human immunoglobulin variable domain that binds a polypeptide antigen with a K_{d} of less than or equal to 100 nM, wherein the polypeptide is present at a concentration of at least 400 μM, and wherein the polypeptide further comprises a covalently linked polymer molecule. In one embodiment, the polypeptide antigen is a human polypeptide antigen.

In one embodiment, the polymer is linked to the polypeptide comprising a single immunoglobulin variable domain via a cysteine or lysine residue comprised by the polypeptide. Due to potential effects on the overall folding or conformation of the variable domain, which in turn can affect the antigen binding affinity or specificity, it is preferred that polymer be attached at or near the amino or carboxy terminus of the variable domain polypeptide. Thus, in another embodiment, the cysteine or lysine residue is present at the C-terminus of the immunoglobulin variable domain polypeptide. In another embodiment, the cysteine or lysine residue has been added to the polypeptide comprising a single immunoglobulin variable domain. In another embodiment, the cysteine or lysine residue has been added at the amino or carboxy terminus of the polypeptide comprising a single immunoglobulin variable domain.

In another embodiment, the polymer comprises a substituted or unsubstituted straight or branched chain polyalkylene, polyalkenylene or polyoxyalkylene polymer or a branched or unbranched polysaccharide.

In another embodiment, the polymer comprises a substituted or unsubstituted straight or branched chain polyethylene glycol or polyvinyl alcohol.

In another embodiment, the polymer comprises methoxy(polyethylene glycol).

In another embodiment, the polymer comprises polyethylene glycol. In one embodiment, the molecular weight of the polyethylene glycol is 5,000 to 50,000 kD.

In another embodiment, the polypeptide has a hydrodynamic size of at least 24 kDa. In another embodiment, the polypeptide has a total PEG size of from 20 to 60 kDa.

In another embodiment, the polypeptide has a hydrodynamic size of at least 200 kDa. In another embodiment, the polypeptide has a total PEG size of from 20 to 60 kDa.

In another embodiment, the PEG-linked polypeptide retains at least 90% activity relative to the same polypeptide lacking the PEG molecule, wherein activity is measured by affinity of the polypeptide for a target ligand.

In one embodiment, the polypeptide has an increased in vivo half-life relative to the same polypeptide composition lacking covalently linked polyethylene glycol.

In another embodiment, the tα-half life of the polypeptide composition is increased by 10% or more. In another embodiment, the tα-half life of the polypeptide composition is increased by 50% or more. In another embodiment, the tα-half life of the polypeptide composition is increased by 2× or more. In another embodiment, the tα-half life of the polypeptide composition is increased by 10× or more. In another embodiment, the tα-half life of the polypeptide composition is increased by 50× or more.

In another embodiment, the tα-half life of the polypeptide composition is in the range of 30 minutes to 12 hours. In another embodiment, the tα-half life of the polypeptide composition is in the range of 1 to 6 hours.

In another embodiment, the tβ-half life of the polypeptide composition is increased by 10% or more. In another embodiment, the tα-half life of the polypeptide composition is increased by 50% or more. In another embodiment, the tα-half life of the polypeptide composition is increased by 2× or more. In another embodiment, the tα-half life of the polypeptide composition is increased by 10× or more. In another embodiment, the tα-half life of the polypeptide composition is increased by 50× or more.

In another embodiment, the tβ-half life is in the range of 12 to 60 hours. In another embodiment, the tβ-half life is in the range of 12 to 26 hours.

In another embodiment, the composition has an AUC value of 15 mg.min/ml to 150 mg.min/ml. In another embodiment, the composition has an AUC value of 15 mg.min/ml to 100 mg.min/ml. In another embodiment, the composition has an AUC value of 15 mg.min/ml to 75 mg.min/ml. In another embodiment, the composition has an AUC value of 15 mg.min/ml to 50 mg.min/ml.

The invention further encompasses a composition comprising a polypeptide comprising a single immunoglobulin V_{L} domain that binds a target antigen with a K_{d} of less than or equal to 100 nM, wherein the polypeptide is present at a concentration of at least 400 μM as determined by absorbance of light at 280 nm wavelength.

In one embodiment, the single immunoglobulin V_{L} domain is a human V_{L} domain.

In another embodiment, the target antigen is a human antigen.

In another embodiment, the composition further comprises a pharmaceutically acceptable carrier.

In another embodiment, the polypeptide comprises a homomultimer of the single immunoglobulin V_{L} domain. In another embodiment, the homomultimer is a homodimer or a homotrimer.

The invention further encompasses extended release parenteral or oral dosage formulations of the single immunoglobulin variable domain polypeptides and preparations described herein. In one embodiment, the dosage formulation is suitable for parenteral administration via a route selected from the group consisting of intravenous, intramuscular or intraperitoneal injection, implantation, rectal and transdermal administration. In another embodiment, implantation comprises intratumor implantation.

The invention further encompasses methods of treating a disease or disorder comprising administering an extended release dosage formulation of a single immunoglobulin variable domain polypeptide preparation as described herein.

### DEFINITIONS

As used herein, the term “domain” refers to a folded protein structure which retains its tertiary structure independently of the rest of the protein. Generally, domains are responsible for discrete functional properties of proteins, and in many cases may be added, removed or transferred to other proteins without loss of function of the remainder of the protein and/or of the domain.

By “single immunoglobulin variable domain” is meant a folded polypeptide domain which comprises sequences characteristic of immunoglobulin variable domains and which specifically binds an antigen (i.e., dissociation constant of 500 nM or less). A “single immunoglobulin variable domain” therefore includes complete antibody variable domains as well as modified variable domains, for example in which one or more loops have been replaced by sequences which are not characteristic of antibody variable domains or antibody variable domains which have been truncated or comprise N- or C-terminal extensions, as well as folded fragments of variable domains which retain a dissociation constant of 500 nM or less (e.g., 450 nM or less, 400 nM or less, 350 nM or less, 300 nM or less, 250 nM or less, 200 nM or less, 150 nM or less, 100 nM or less) and the target antigen specificity of the full-length domain. A “domain antibody” or “dAb” is equivalent to a “single immunoglobulin variable domain polypeptide” as the term is used herein.

The phrase “single immunoglobulin variable domain polypeptide” encompasses not only an isolated single immunoglobulin variable domain polypeptide, but also larger polypeptides that comprise one or more monomers of a single immunoglobulin variable domain polypeptide sequence. Such larger polypeptides comprising more thanone monomer of a single immunoglobulin variable domain polypeptide are in noted contrast to scFv polypeptides which comprise a V_{H} and a V_{L} domain that cooperatively bind an antigen molecule. The monomers in the polypeptides described herein can bind antigen independently of each other.

As used herein, the phrase “sequence characteristic of immunoglobulin variable domains” refers to an amino acid sequence that is homologous, over 20 or more (i.e., over at least 20), 25 or more, 30 or more, 35 or more, 40 or more, 45 or more, or even 50 or more contiguous amino acids, to a sequence comprised by an immunoglobulin variable domain sequence.

As used herein, the terms “homology” or “similarity” refer to the degree with which two nucleotide or amino acid sequences structurally resemble each other. As used herein, sequence “similarity” is a measure of the degree to which amino acid sequences share similar amino acid residues at corresponding positions in an alignment of the sequences. Amino acids are similar to each other where their side chains are similar. Specifically, “similarity” encompasses amino acids that are conservative substitutes for each other. A “conservative” substitution is any substitution that has a positive score in the blosum62 substitution matrix (Hentikoff and Hentikoff, 1992, Proc. Natl. Acad. Sci. USA 89: 10915-10919). By the statement “sequence A is n % similar to sequence B” is meant that n % of the positions of an optimal global alignment between sequences A and B consists of identical amino acids or conservative substitutions. Optimal global alignments can be performed using the following parameters in the Needleman-Wunsch alignment algorithm:

For polypeptides:
Substitution matrix: blosum62.
Gap scoring function: −A−B*LG, where A=11 (the gap penalty), B=1 (the gap length penalty) and LG is the length of the gap.

For nucleotide sequences:
Substitution matrix: 10 for matches, 0 for mismatches.
Gap scoring function: −A−B*LG where A=50 (the gap penalty), B=3 (the gap length penalty) and LG is the length of the gap.

Typical conservative substitutions are among Met, Val, Leu and Ile; among Ser and Thr; among the residues Asp, Glu and Asn; among the residues Gln, Lys and Arg; or aromatic residues Phe and Tyr.

As used herein, two sequences are “homologous” or “similar” to each other where they have at least 85% sequence similarity to each other when aligned using either the Needleman-Wunsch algorithm or the “BLAST 2 sequences” algorithm described by Tatusova & Madden, 1999, FEMS Microbiol Lett. 174:247-250. Where amino acid sequences are aligned using the “BLAST 2 sequences algorithm,” the Blosum 62 matrix is the default matrix.

As used herein, the terms “low stringency,” “medium stringency,” “high stringency,” or “very high stringency conditions” describe conditions for nucleic acid hybridization and washing. Guidance for performing hybridization reactions can be found in Current Protocols in Molecular Biology, John Wiley & Sons, N.Y. (1989), 6.3.1-6.3.6, which is incorporated herein by reference in its entirety. Aqueous and nonaqueous methods are described in that reference and either can be used. Specific hybridization conditions referred to herein are as follows: (1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); (2) medium stringency hybridization conditions in 6×SSC at about 45° C., followed by one or more washes in 0.2×SSC, 0.1% SDS at 60° C.; (3) high stringency hybridization conditions in 6×SSC at about 45° C., followed by one or more washes in 0.2×SSC, 0.1% SDS at 65° C.; and preferably (4) very high stringency hybridization conditions are 0.5M sodium phosphate, 7% SDS at 65° C., followed by one or more washes at 0.2×SSC, 1% SDS at 65° C.

As used herein, the phrase “specifically binds” refers to the binding of an antigen by an immunoglobulin variable domain with a dissociation constant (K_{d}) of 1 μM or lower as measured by surface plasmon resonance analysis using, for example, a BIAcore™ surface plasmon resonance system and BIAcore™ kinetic evaluation software (e.g., version 2.1). The affinity or K_{d} for a specific binding interaction is preferably about 500 nM or lower, more preferably about 300 nM or lower.

As used herein, the term “high affinity binding” refers to binding with a K_{d} of less than or equal to 100 nM.

As used herein, the phrase “human immunoglobulin variable domain” refers to a polypeptide having a sequence derived from a human germline immunoglobulin V region. A sequence is “derived from a human germline V region” when the sequence is either isolated from a human individual, isolated from a library of cloned human antibody gene sequences (or a library of human antibody V region gene sequences), or when a cloned human germline V region sequence was used to generate one or more diversified sequences (by random or targeted mutagenesis) that were then selected for binding to a desired target antigen. At a minimum, a human immunoglobulin variable domain has at least 85% amino acid similarity (including, for example, 87%, 90%, 93%, 95%, 97%, 99% or higher similarity) to a naturally-occurring human immunoglobulin variable domain sequence.

Alternatively, or in addition, “a human immunoglobulin variable domain” is a variable domain that comprises four human immunoglobulin variable domain framework regions (FW1-FW4), as framework regions are set forth by Kabat et al. (1991, supra). The “human immunoglobulin variable domain framework regions” encompass a) an amino acid sequence of a human framework region, and b) a framework region that comprises at least 8 contiguous amino acids of the amino acid sequence of a human framework region. A human immunoglobulin variable domain can comprise amino acid sequences of FW1-FW4 that are the same as the amino acid sequences of corresponding framework regions encoded by a human germline antibody gene segment, or it can also comprise a variable domain in which FW1-FW4 sequences collectively contain up to 10 amino acid sequence differences (e.g., up to 1, 2, 3, 4, 5, 6, 7, 8, 9 or 10 amino acid sequence differences) relative to the amino acid sequences of corresponding framework regions encoded by a human germline antibody gene segment.

A “human immunoglobulin variable domain” as defined herein has the capacity to specifically bind an antigen on its own, whether the variable domain is present as a single immunoglobulin variable domain alone, or as a single immunoglobulin variable domain in association with one or more additional polypeptide sequences. A “human immunoglobulin variable domain” as the term is used herein does not encompass a “humanized” immunoglobulin polypeptide, i.e., a non-human (e.g., mouse, camel, etc.) immunoglobulin that has been modified in the constant regions to render it less immunogenic in humans.

As used herein, the phrase “at a concentration of” means that a given polypeptide is dissolved in solution (preferably aqueous solution) at the recited mass or molar amount per unit volume. A polypeptide that is present “at a concentration of X” or “at a concentration of at least X” is therefore exclusive of both dried and crystallized preparations of a polypeptide.

As used herein, the term “repertoire” refers to a collection of diverse variants, for example polypeptide variants which differ in their primary sequence. A library used in the present invention will encompass a repertoire of polypeptides comprising at least 1000 members.

As used herein, the term “library” refers to a mixture of heterogeneous polypeptides or nucleic acids. The library is composed of members, each of which have a single polypeptide or nucleic acid sequence. To this extent, library is synonymous with repertoire. Sequence differences between library members are responsible for the diversity present in the library. The library may take the form of a simple mixture of polypeptides or nucleic acids, or may be in the form of organisms or cells, for example bacteria, viruses, animal or plant cells and the like, transformed with a library of nucleic acids. Preferably, each individual organism or cell contains only one or a limited number of library members. Advantageously, the nucleic acids are incorporated into expression vectors, in order to allow expression of the polypeptides encoded by the nucleic acids. In a preferred aspect, therefore, a library may take the form of a population of host organisms, each organism containing one or more copies of an expression vector containing a single member of the library in nucleic acid form which can be expressed to produce its corresponding polypeptide member. Thus, the population of host organisms has the potential to encode a large repertoire of genetically diverse polypeptide variants.

As used herein, the term “antigen” refers to a molecule that is bound by an antibody or a binding region (e.g., a variable domain) of an antibody. Typically, antigens are capable of raising an antibody response in vivo. An antigen can be a peptide, polypeptide, protein, nucleic acid, lipid, carbohydrate, or other molecule. Generally, an immunoglobulin variable domain is selected for target specificity against a particular antigen.

As used herein, the term “epitope” refers to a unit of structure conventionally bound by an immunoglobulin V_{H}/V_{L} pair. Epitopes define the minimum binding site for an antibody, and thus represent the target of specificity of an antibody. In the case of a single domain antibody, an epitope represents the unit of structure bound by a variable domain in isolation.

As used herein, the term “neutralizing,” when used in reference to a single immunoglobulin variable domain polypeptide as described herein, means that the polypeptide interferes with a measurable activity or function of the target antigen. A polypeptide is a “neutralizing” polypeptide if it reduces a measurable activity or function of the target antigen by at least 50%, and preferably at least 60%, 70%, 80%, 90%, 95% or more, up to and including 100% inhibition (i.e., no detectable effect or function of the target antigen). This reduction of a measurable activity or function of the target antigen can be assessed by one of skill in the art using standard methods of measuring one or more indicators of such activity or function. As an example, where the target is TNF-α, neutralizing activity can be assessed using a standard L929 cell killing assay or by measuring the ability of a single immunoglobulin variable domain to inhibit TNF-α-induced expression of ELAM-1 on HUVEC, which measures TNF-α-induced cellular activation.

As used herein, a “measurable activity or function of a target antigen” includes, but is not limited to, for example, cell signaling, enzymatic activity, binding activity, ligand-dependent internalization, cell killing, cell activation, promotion of cell survival, and gene expression. One of skill in the art can perform assays that measure such activities for a given target antigen.

As used herein, the term “agonist” when used in reference to a single immunoglobulin variable domain polypeptide as described herein means that the polypeptide enhances or activates a measurable function or activity of the target antigen. For example, when a single immunoglobulin variable domain that binds a cell surface receptor activates intracellular signaling by the receptor, enhances binding or signaling by a natural ligand, or enhances internalization of the receptor/ligand complex, the variable domain polypeptide is an agonist. An agonist causes an increase in a measurable activity of its target antigen by at least 50% relative to the absence of the agonist or, alternatively, relative to the increase caused by a natural ligand of the target antigen, and preferably at least 2-fold, 3-fold, 5-fold, 10-fold, 20-fold, 50-fold, 100-fold or more above such activity.

As used herein, the terms “homodimer,” “homotrimer”, “homotetramer”, and “homomultimer” refer to molecules comprising two, three or more (e.g., four, five, etc.) monomers of a given single immunoglobulin variable domain polypeptide sequence, respectively. For example, a homodimer could include two copies of the same V_{H} sequence. A “monomer” of a single immunoglobulin variable domain polypeptide is a single V_{H} or V_{L} sequence that specifically binds antigen. The monomers in a homodimer, homotrimer, homotetramer, or homomultimer can be linked either by expression as a fusion polypeptide, e.g., with a peptide linker between monomers, or, by chemically joining monomers after translation either to each other directly or through a linker by disulfide bonds, or by linkage to a di-, tri- or multivalent linking moiety. In one embodiment, the monomers in a homodimer, trimer, tetramer, or multimer can be linked by a multi-arm PEG polymer, wherein each monomer of the dimer, trimer, tetramer, or multimer is linked to a PEG moiety of the multi-arm PEG.

As used herein, the terms “heterodimer,” “heterotrimer” and “hetero-multimer” refer to molecules comprising two, three, or more (e.g., four, five, etc.) single immunoglobulin variable domains wherein at least one single immunoglobulin variable domain binds a different antigen than the other(s). For example, a heterodimer could comprise a single immunoglobulin V_{H} domain polypeptide that binds a given antigen, fused to another immunoglobulin V domain (e.g., another V_{H} domain) that binds a different antigen. The individual binding domains (monomers) can be linked together through expression as a fusion protein, either directly or through a peptide linker, or they can be chemically linked as described above for homomultimers. Likewise, the “monomers” in the heteromultimer can also be linked through expression as a single polypeptide or by chemical linkage.

As used herein, the term “polymer molecule” refers to a chemical moiety formed by the covalent chemical union of two or more (i.e., 3 or more, 4 or more, preferably 5, 10, 20, 50, 70, 90, 100 or more, often many more, e.g., 1000 or more) identical combining units. As the term is used herein, the term “polymer molecule” specifically excludes polypeptides or nucleic acids which are often referred to in the art as polymers—thus, a polypeptide fused to another polypeptide is not a polypeptide fused to a polymer. The term “polymer molecule” also encompasses co-polymer molecules.

As used herein, the term “half-life” refers to the time taken for the serum concentration of a ligand (e.g., a single immunoglobulin variable domain) to reduce by 50%, in vivo, for example due to degradation of the ligand and/or clearance or sequestration of the ligand by natural mechanisms. The ligands of the invention are stabilised in vivo and their half-life increased by binding to molecules which resist degradation and/or clearance or sequestration. Typically, such molecules are naturally occurring proteins which themselves have a long half-life in vivo. The half-life of a ligand is increased if its functional activity persists, in vivo, for a longer period than a similar ligand which is not specific for the half-life increasing molecule. Thus, a ligand specific for HSA and a target molecule is compared with the same ligand wherein the specificity for HSA is not present—it does not bind HSA but binds another molecule. For example, it may bind a second epitope on the target molecule. Typically, the half life is increased by 10%, 20%, 30%, 40%, 50% or more. Increases in the range of 2×, 3×, 4×, 5×, 10×, 20×, 30×, 40×, 50× or more of the half life are possible. Alternatively, or in addition, increases in the range of up to 30×, 40×, 50×, 60×, 70×, 80×, 90×, 100×, 150× of the half life are possible.

As used herein, the term “extended release” or the equivalent terms “controlled release” or “slow release” refer to drug formulations that release active drug, such as a polypeptide drug, over a period of time following administration to an individual. Extended release of polypeptide drugs, which can occur over a range of times, e.g., minutes, hours, days, weeks or longer, depending upon the drug formulation, is in contrast to standard formulations in which substantially the entire dosage unit is available for immediate absorption or immediate distribution via the bloodstream. Preferred extended release formulations result in a level of circulating drug from a single administration that is sustained, for example, for 8 hours or more, 12 hours or more, 24 hours or more, 36 hours or more, 48 hours or more, 60 hours or more, 72 hours or more 84 hours or more, 96 hours or more, or even, for example, for 1 week or 2 weeks or more, for example, 1 month or more.

As used herein, the phrase “generic ligand” refers to a ligand that binds to all members of a repertoire. A generic ligand is generally not bound through the antigen binding site of an antibody or variable domain. Non-limiting examples of generic ligands include protein A and protein L.

As used herein, the phrase “universal framework” refers to a single antibody framework sequence corresponding to the regions of an antibody conserved in sequence as defined by Kabat et al. (1991, supra) or corresponding to the human germline immunoglobulin repertoire or structure as defined by Chothia and Lesk, (1987) J. Mol. Biol. 196:910-917. The invention provides for the use of a single framework, or a set of such frameworks, which has been found to permit the derivation of virtually any binding specificity though variation in the hypervariable regions alone.

### BRIEF DESCRIPTION OF THE FIGURES

FIG. 1 shows the sequence of the dummy V_{H} diversified to generate library 1. The sequence is the V_{H} framework based on germline sequence DP47-JH4b. Positions where NNK randomization (N=A or T or C or G nucleotides; K=G or T nucleotides) has been incorporated into library 1 are indicated in bold underlined text. HCDRs 1-3 are indicated by underlining.

FIG. 2 shows the sequence of the dummy V_{H} diversified to generate library 2. The sequence is the V_{H} framework based on germline sequence DP47-JH4b. Positions where NNK randomization (N=A or T or C or G nucleotides; K=G or T nucleotides) has been incorporated into library 2 are indicated in bold underlined text. HCDRs 1-3 are indicated by underlining.

FIG. 3 shows the sequence of dummy Vκ diversified to generate library 3. The sequence is the V_{κ} framework based on germline sequence DP_{K}9-J_{K}1. Positions where NNK randomization (N=A or T or C or G nucleotides; K=G or T nucleotides) has been incorporated into library 3 are indicated in bold underlined text. LCDRs 1-3 are indicated by underlining.

FIG. 4 shows nucleotide and amino acid sequence of anti MSA dAbs MSA 16 and MSA 26.

FIGS. 5 and 6 show SPR analysis of MSA 16 and 26. Purified dAbs MSA16 and MSA26 were analysed by inhibition BIAcore™ surface plasmon resonance analysis to determine K_{d}. Briefly, the dAbs were tested to determine the concentration of dAb required to achieve 200RUs of response on a BIAcore CM5™ chip coated with a high density of MSA. Once the required concentrations of dAb had been determined, MSA antigen at a range of concentrations around the expected K_{d} was premixed with the dAb and incubated overnight. Binding of dAb to the MSA coated BIAcore™ chip in each of the premixes was then measured at a high flow-rate of 30 μl/minute.

FIG. 7 shows serum levels of MSA16 following injection. Serum half life of the dAb MSA16 was determined in mouse. MSA16 was dosed as single i.v. injections at approx 1.5 mg/kg into CD1 mice. Modeling with a 2 compartment model showed MSA16 had a t1/2α of 0.98 hr, a t1/2β of 36.5 hr and an AUC of 913 hr.mg/ml. MSA16 had a considerably lengthened half life compared with HEL4 (an anti-hen egg white lysozyme dAb) which had a t1/2α of 0.06 hr and a t1/2β of 0.34 hr.

FIG. 8 shows nucleotide and amino acid sequences of single immunoglobulin variable domain polypeptides HEL4 (binds hen egg lysozyme), TAR1-5-19 (binds TNF-α), and TAR2 (binds p55 TNFR).

FIG. 9 shows the results of a TNF receptor assay comparing TAR 1-5 dimers 1-6.

FIG. 10 shows the results of a TNF receptor assay comparing TAR1-5 dimer 4, TAR1-5-19 dimer 4 and TAR1-5-19 monomer.

FIG. 11 shows the results if a TNF receptor assay of TAR1-5-19 homodimers in different formats: dAb-linker-dAb format with 3U, 5U or 7U linker, Fab format and cysteine hinge linker format.

FIG. 12 shows the sequences of single immunoglobulin variable domains described in Example 5.

FIG. 13 shows a graph of the results of solubility studies of the anti-TNF-α dAb TAR1-5-19 under different buffer conditions. “Obs” is the observed concentration achieved at the various volumes shown, and “exp” is the expected concentration based on the amount of starting material.

FIG. 14 shows a graph of the results of solubility studies of the anti-TNFR1 dAb TAR2h-10-27 under different buffer conditions: Tar2a=TAR2h-10-27-cys reduced in Tris/Glycine plus 10% glycerol, pH4; Tar2b=TAR2h-10-27 wt in Tris/Glycine plus 10% glycerol, pH7; Tar2c=TAR2h-10-27Cys PEG 2×10 K in 50 mM Tris Acetate, pH4; Tar2d=TAR2h-10-27 wt in Tris/Glycine plus 10% glycerol, pH5; Tar2e=TAR2h-10-27Cys in 50 mM Tris Acetate, blocked i.e. non-PEGylated, and Tar2f=TAR2h-10-27Cys reduced in PBS, pH 7.2. “Obs” is the observed concentration achieved at the various volumes shown, and “exp” is the expected concentration based on the amount of starting material. Differences between observed and expected values indicate, in part, whether loss has occurred due to precipitation.

FIG. 15 shows the polynucleotide and amino acid sequences for the TAR2h-10-27 anti-TNFR1 dAb. It is noted that position 103 (Kabat numbering convention) is an arginine residue.

FIG. 16 shows the polynucleotide and amino acid sequences for the TAR4-10 and TAR4-116 anti-CD40L dAbs.

### DETAILED DESCRIPTION OF THE INVENTION

The invention relates to polypeptides comprising single immunoglobulin variable domains or multimers of such domains that have high binding affinity for specific target molecules or antigens. The invention also relates to high molarity preparations of such polypeptides. Single immunoglobulin V_{H} domains from camelid species (V_{H}H) are known to possess high affinity binding capacity and to be highly soluble relative to V domains of non-camelid species. However, camelid antibodies have limited therapeutic potential because they are themselves antigenic when administered to non-camelid individuals, e.g., humans. The invention provides human single immunoglobulin variable domains that possess high binding affinity and high solubility. These V domains are both V_{H} and V_{L} domains.

The invention also relates to V_{L} single immunoglobulin variable domains that possess high binding affinity and high solubility, and to V domain polypeptides modified to have high solubility, e.g., by alteration of V_{H} residues at positions 44, 45, 47 and 103 per the Kabat numbering convention.

### Preparation of Human Single Immunoglobulin Variable Domains:

Human single immunoglobulin variable domains are prepared in a number of ways. For each of these approaches, well-known methods of preparing (e.g., amplifying, mutating, etc.) and manipulating nucleic acid sequences are applicable.

One means is to amplify and express the V_{H} or V_{L} region of a heavy chain or light chain gene for a cloned antibody known to bind the desired antigen. The boundaries of V_{H} and V_{L} domains are set out by Kabat et al. (1991, supra). The information regarding the boundaries of the V_{H} and V_{L} domains of heavy and light chain genes is used to design PCR primers that amplify the V domain from a cloned heavy or light chain coding sequence encoding an antibody known to bind a given antigen. The amplified V domain is inserted into a suitable expression vector, e.g., pHEN-1 (Hoogenboom et al., 1991, Nucleic Acids Res. 19: 4133-4137) and expressed, either alone or as a fusion with another polypeptide sequence. The expressed V_{H} or V_{L} domain is then screened for high affinity binding to the desired antigen in isolation from the remainder of the heavy or light chain polypeptide. For all aspects of the present invention, screening for binding is performed as known in the art or as described herein below.

A repertoire of V_{H} or V_{L} domains is screened by, for example, phage display, panning against the desired antigen. Methods for the construction of bacteriophage display libraries and lambda phage expression libraries are well known in the art, and taught, for example, by: McCafferty et al., 1990, Nature 348: 552; Kang et al., 1991, Proc. Natl. Acad. Sci. U.S.A., 88: 4363; Clackson et al., 1991, Nature 352: 624; Lowman et al., 1991, Biochemistry 30: 10832; Burton et al., 1991, Proc. Natl. Acad. Sci. U.S.A. 88: 10134; Hoogenboom et al., 1991, Nucleic Acids Res. 19: 4133; Chang et al., 1991, J. Immunol. 147: 3610; Breitling et al., 1991, Gene 104: 147; Marks et al., 1991, J. Mol. Biol. 222: 581; Barbas et al., 1992, Proc. Natl. Acad. Sci. U.S.A. 89: 4457; Hawkins and Winter (1992) J. Immunol., 22: 867; Marks et al. (1992) J. Biol. Chem., 267: 16007; and Lerner et al. (1992) Science, 258: 1313. scFv phage libraries are taught, for example, by Huston et al., 1988, Proc. Natl. Acad. Sci. U.S.A. 85: 5879-5883; Chaudhary et al., 1990, Proc. Natl. Acad. Sci. U.S.A. 87: 1066-1070; McCafferty et al., 1990, supra; Clackson et al., 1991, supra; Marks et al., 1991, supra; Chiswell et al., 1992, Trends Biotech. 10: 80; and Marks et al., 1992, supra. Various embodiments of scFv libraries displayed on bacteriophage coat proteins have been described. Refinements of phage display approaches are also known, for example as described in WO96/06213 and WO92/01047 (Medical Research Council et al.) and WO97/08320 (Morphosys, supra).

The repertoire of V_{H} or V_{L} domains can be a naturally-occurring repertoire of immunoglobulin sequences or a synthetic repertoire. A naturally-occurring repertoire is one prepared, for example, from immunoglobulin-expressing cells harvested from one or more individuals. Such repertoires can be “naïve,” i.e., prepared, for example, from human fetal or newborn immunoglobulin-expressing cells, or rearranged, i.e., prepared from, for example, adult human B cells. Natural repertoires are described, for example, by Marks et al., 1991, J. Mol. Biol. 222: 581 and Vaughan et al., 1996, Nature Biotech. 14: 309. If desired, clones identified from a natural repertoire, or any repertoire, for that matter, that bind the target antigen are then subjected to mutagenesis and further screening in order to produce and select variants with improved binding characteristics.

Synthetic repertoires of single immunoglobulin variable domains are prepared by artificially introducing diversity into a cloned V domain. Synthetic repertoires are described, for example, by Hoogenboom & Winter, 1992, J. Mol. Biol. 227: 381; Barbas et al., 1992, Proc. Natl. Acad. Sci. U.S.A. 89: 4457; Nissim et al., 1994, EMBO J. 13: 692; Griffiths et al., 1994, EMBO J. 13: 3245; DeKriuf et al., 1995, J. Mol. Biol. 248: 97; and WO 99/20749.

The antigen binding domain of a conventional antibody comprises two separate regions: a heavy chain variable domain (V_{H}) and a light chain variable domain (V_{L}: which can be either V_{κ} or V_{λ}). The antigen binding site of such an antibody is formed by six polypeptide loops: three from the V_{H} domain (H1, H2 and H3) and three from the V_{L} domain (L1, L2 and L3). The boundaries of these loops are described, for example, in Kabat et al. (1991, supra). A diverse primary repertoire of V genes that encode the V_{H} and V_{L} domains is produced in vivo by the combinatorial rearrangement of gene segments. The V_{H} gene is produced by the recombination of three gene segments, V_{H}, D and J_{H}. In humans, there are approximately 51 functional V_{H} segments (Cook and Tomlinson (1995) Immunol Today 16: 237), 25 functional D segments (Corbett et al. (1997) J. Mol. Biol. 268: 69) and 6 functional J_{H} segments (Ravetch et al. (1981) Cell 27: 583), depending on the haplotype. The V_{H} segment encodes the region of the polypeptide chain which forms the first and second antigen binding loops of the V_{H} domain (H1 and H2), while the V_{H}, D and J_{H} segments combine to form the third antigen binding loop of the V_{H} domain (H3).

The V_{L} gene is produced by the recombination of only two gene segments, V_{L} and J_{L}. In humans, there are approximately 40 functional V_{κ} segments (Schäble and Zachau (1993) Biol. Chem. Hoppe-Seyler 374: 1001), 31 functional V_{λ} segments (Williams et al. (1996) J. Mol. Biol. 264: 220; Kawasaki et al. (1997) Genome Res. 7: 250), 5 functional J_{κ} segments (Hieter et al. (1982) J. Biol. Chem. 257: 1516) and 4 functional J_{λ} segments (Vasicek and Leder (1990) J. Exp. Med. 172: 609), depending on the haplotype. The V_{L} segment encodes the region of the polypeptide chain which forms the first and second antigen binding loops of the V_{L} domain (L1 and L2), while the V_{L} and J_{L} segments combine to form the third antigen binding loop of the V_{L} domain (L3). Antibodies selected from this primary repertoire are believed to be sufficiently diverse to bind almost all antigens with at least moderate affinity. High affinity antibodies are produced in vivo by “affinity maturation” of the rearranged genes, in which point mutations are generated and selected by the immune system on the basis of improved binding.

Analysis of the structures and sequences of antibodies has shown that five of the six antigen binding loops (H1, H2, L1, L2, L3) possess a limited number of main-chain conformations or canonical structures (Chothia and Lesk (1987) J. Mol. Biol. 196: 901; Chothia et al. (1989) Nature 342: 877). The main-chain conformations are determined by (i) the length of the antigen binding loop, and (ii) particular residues, or types of residue, at certain key position in the antigen binding loop and the antibody framework. Analysis of the loop lengths and key residues has enabled us to the predict the main-chain conformations of H1, H2, L1, L2 and L3 encoded by the majority of human antibody sequences (Chothia et al. (1992) J. Mol. Biol. 227: 799; Tomlinson et al. (1995) EMBO J. 14: 4628; Williams et al. (1996) J. Mol. Biol. 264: 220). Although the H3 region is much more diverse in terms of sequence, length and structure (due to the use of D segments), it also forms a limited number of main-chain conformations for short loop lengths which depend on the length and the presence of particular residues, or types of residue, at key positions in the loop and the antibody framework (Martin et al. (1996) J. Mol. Biol. 263: 800; Shirai et al. (1996) FEBS Letters 399: 1.

While, according to one embodiment of the invention, diversity can be added to synthetic repertoires at any site in the CDRs of the various antigen-binding loops, this approach results in a greater proportion of V domains that do not properly fold and therefore contribute to a lower proportion of molecules with the potential to bind antigen. An understanding of the residues contributing to the main chain conformation of the antigen-binding loops permits the identification of specific residues to diversify in a synthetic repertoire of V_{H} or V_{L} domains. That is, diversity is best introduced in residues that are not essential to maintaining the main chain conformation. As an example, for the diversification of loop L2, the conventional approach would be to diversify all the residues in the corresponding CDR (CDR2) as defined by Kabat et al. (1991, supra), some seven residues. However, for L2, it is known that positions 50 and 53 are diverse in naturally occurring antibodies and are observed to make contact with the antigen. The preferred approach would be to diversify only those two residues in this loop. This represents a significant improvement in terms of the functional diversity required to create a range of antigen binding specificities.

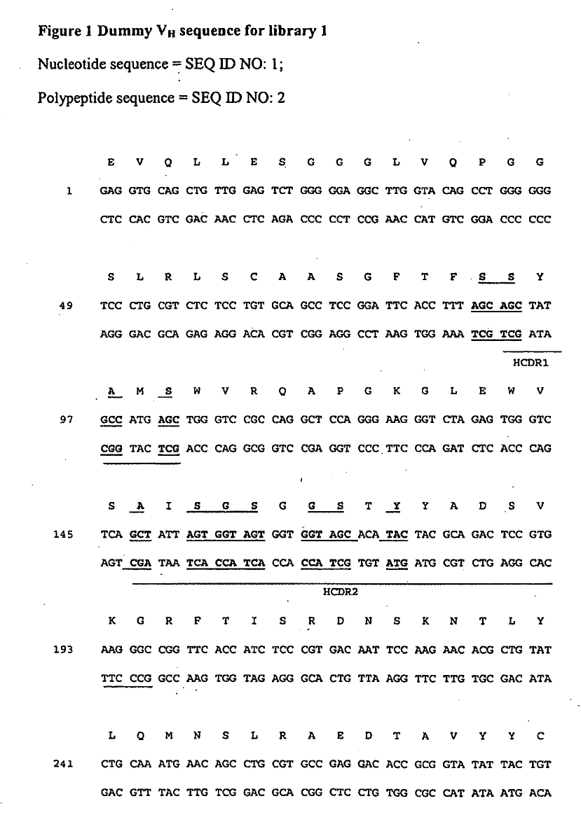

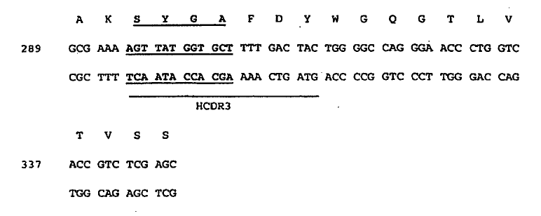

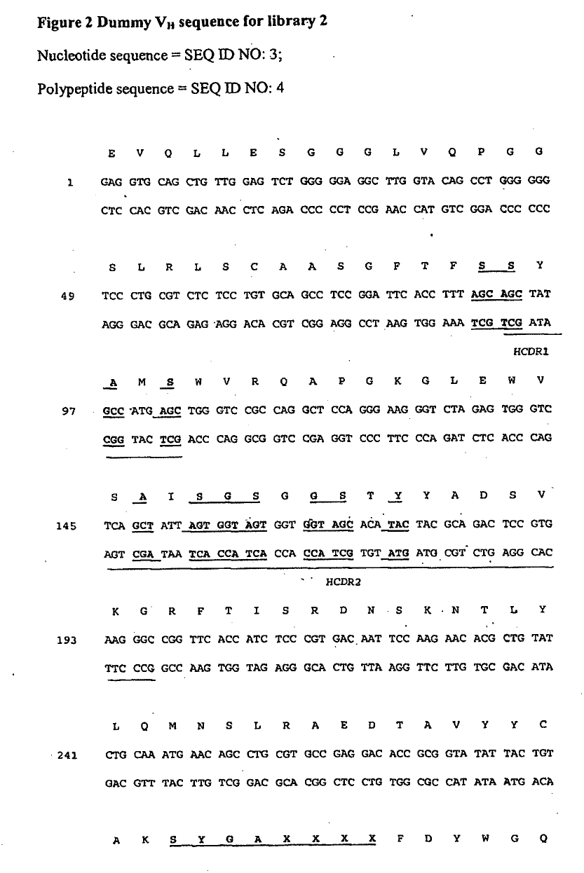

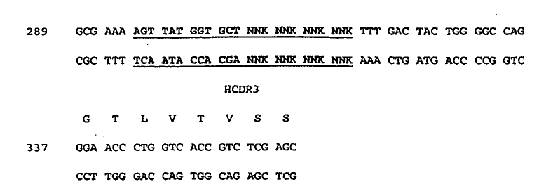

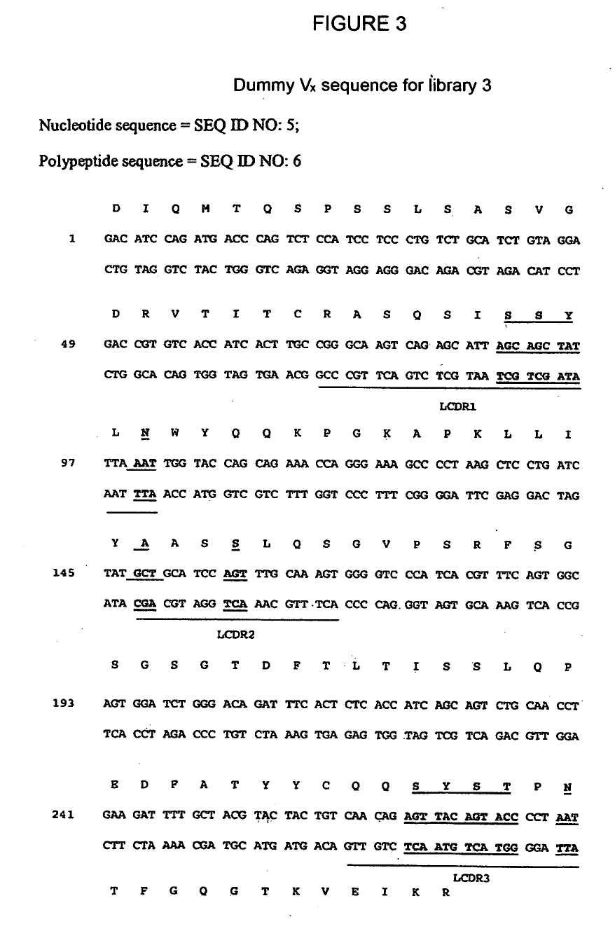

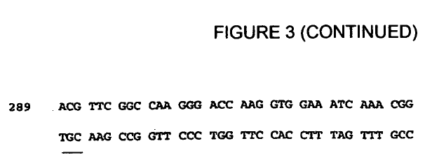

In one aspect, synthetic variable domain repertoires are prepared in V_{H} or Vκ backgrounds, based on artificially diversified germline V_{H} or Vκ sequences. For example, the V_{H} domain repertoire is based on cloned germline V_{H} gene segments V3-23/DP47 (Tomlinson et al., 1992, J. Mol. Biol. 227: 7768) and JH4b (see FIGS. 1 and 2). The V_{κ} domain repertoire is based, for example, on germline V_{κ} gene segments O2/O12/DPK9 (Cox et al., 1994, Eur. J. Immunol. 24: 827) and J_{κ}1 (see FIG. 3). Diversity is introduced into these or other gene segments by, for example, PCR mutagenesis. Diversity can be randomly introduced, for example, by error prone PCR (Hawkins, et al., 1992, J. Mol. Biol. 226: 889) or chemical mutagenesis. As discussed above, however it is preferred that the introduction of diversity is targeted to particular residues. It is further preferred that the desired residues are targeted by introduction of the codon NNK using mutagenic primers (using the IUPAC nomenclature, where N=G, A, T or C, and K=G or T), which encodes all amino acids and the TAG stop codon. Other codons which achieve similar ends are also of use, including the NNN codon (which leads to the production of the additional stop codons TGA and TAA), DVT codon ((A/G/T) (A/G/C)T), DVC codon ((A/G/T)(A/G/C)C), and DVY codon ((A/G/T)(A/G/C)(C/T). The DVT codon encodes 22% serine and 11% tyrosine, aspargine, glycine, alanine, aspartate, threonine and cysteine, which most closely mimics the distribution of amino acid residues for the antigen binding sites of natural human antibodies. Repertoires are made using PCR primers having the selected degenerate codon or codons at each site to be diversified. PCR mutagenesis is well known in the art; however, considerations for primer design and PCR mutagenesis useful in the methods of the invention are discussed below in the section titled “PCR Mutagenesis.”

In one aspect, diversity is introduced into the sequence of human germline V_{H} gene segments V3-23/DP47 (Tomlinson et al., 1992, J. Mol. Biol. 227: 7768) and JH4b using the NNK codon at sites H30, H31, H33, H35, H50, H52, H52a, H53, H55, H56, H58, H95, H97 and H98, corresponding to diversity in CDRs 1, 2 and 3, as shown in FIG. 1.

In another aspect, diversity is also introduced into the sequence of human germline V_{H} gene segments V3-23/DP47 and JH4b, for example, using the NNK codon at sites H30, H31, H33, H35, H50, H52, H52a, H53, H55, H56, H58, H95, H97, H98, H99, H100, H100a and H100b, corresponding to diversity in CDRs 1, 2 and 3, as shown in FIG. 2.

In another aspect, diversity is introduced into the sequence of human germline V_{κ} gene segments O2/O12/DPK9 and J_{κ}1, for example, using the NNK codon at sites L30, L31, L32, L34, L50, L53, L91, L92, L93, L94 and L96, corresponding to diversity in CDRs 1, 2 and 3, as shown in FIG. 3.

Diversified repertoires are cloned into phage display vectors as known in the art and as described, for example, in WO 99/20749. In general, the nucleic acid molecules and vector constructs required for the performance of the present invention are available in the art and are constructed and manipulated as set forth in standard laboratory manuals, such as Sambrook et al. (1989). Molecular Cloning: A Laboratory Manual, Cold Spring Harbor, USA.

The manipulation of nucleic acids in the present invention is typically carried out in recombinant vectors. As used herein, “vector” refers to a discrete element that is used to introduce heterologous DNA into cells for the expression and/or replication thereof. Methods by which to select or construct and, subsequently, use such vectors are well known to one of skill in the art. Numerous vectors are publicly available, including bacterial plasmids, bacteriophage, artificial chromosomes and episomal vectors. Such vectors may be used for simple cloning and mutagenesis; alternatively, as is typical of vectors in which repertoire (or pre-repertoire) members of the invention are carried, a gene expression vector is employed. A vector of use according to the invention is selected to accommodate a polypeptide coding sequence of a desired size, typically from 0.25 kilobase (kb) to 40 kb in length. A suitable host cell is transformed with the vector after in vitro cloning manipulations. Each vector contains various functional components, which generally include a cloning (or “polylinker”) site, an origin of replication and at least one selectable marker gene. If a given vector is an expression vector, it additionally possesses one or more of the following: enhancer element, promoter, transcription termination and signal sequences, each positioned in the vicinity of the cloning site, such that they are operatively linked to the gene encoding a polypeptide repertoire member according to the invention.

Both cloning and expression vectors generally contain nucleic acid sequences that enable the vector to replicate in one or more selected host cells. Typically in cloning vectors, this sequence is one that enables the vector to replicate independently of the host chromosomal DNA and includes origins of replication or autonomously replicating sequences. Such sequences are well known for a variety of bacteria, yeast and viruses. The origin of replication from the plasmid pBR322 is suitable for most Gram-negative bacteria, the 2 micron plasmid origin is suitable for yeast, and various viral origins (e.g. SV 40, adenovirus) are useful for cloning vectors in mammalian cells. Generally, the origin of replication is not needed for mammalian expression vectors unless these are used in mammalian cells able to replicate high levels of DNA, such as COS cells.

Advantageously, a cloning or expression vector also contains a selection gene also referred to as selectable marker. This gene encodes a protein necessary for the survival or growth of transformed host cells grown in a selective culture medium. Host cells not transformed with the vector containing the selection gene will therefore not survive in the culture medium. Typical selection genes encode proteins that confer resistance to antibiotics and other toxins, e.g. ampicillin, neomycin, methotrexate or tetracycline, complement auxotrophic deficiencies, or supply critical nutrients not available in the growth media.

Because the replication of vectors according to the present invention is most conveniently performed in E. coli, an E. coli-selectable marker, for example, the β-lactamase gene that confers resistance to the antibiotic ampicillin, is of use. These can be obtained from E. coli plasmids, such as pBR322 or a pUC plasmid such as pUC18 or pUC19.

Expression vectors usually contain a promoter that is recognized by the host organism and is operably linked to the coding sequence of interest. Such a promoter may be inducible or constitutive. The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences.

Promoters suitable for use with prokaryotic hosts include, for example, the β-lactamase and lactose promoter systems, alkaline phosphatase, the tryptophan (trp) promoter system and hybrid promoters such as the tac promoter. Promoters for use in bacterial systems will also generally contain a Shine-Dalgarno sequence operably linked to the coding sequence.

In libraries or repertoires as described herein, the preferred vectors are expression vectors that enable the expression of a nucleotide sequence corresponding to a polypeptide library member. Thus, selection is performed by separate propagation and expression of a single clone expressing the polypeptide library member or by use of any selection display system. As described above, a preferred selection display system uses bacteriophage display. Thus, phage or phagemid vectors can be used. Preferred vectors are phagemid vectors, which have an E. coli origin of replication (for double stranded replication) and also a phage origin of replication (for production of single-stranded DNA). The manipulation and expression of such vectors is well known in the art (Hoogenboom and Winter (1992) supra; Nissim et al. (1994) supra). Briefly, the vector contains a β-lactamase or other selectable marker gene to confer selectivity on the phagemid, and a lac promoter upstream of a expression cassette that consists (N to C terminal) of a pelB leader sequence (which directs the expressed polypeptide to the periplasmic space), a multiple cloning site (for cloning the nucleotide version of the library member), optionally, one or more peptide tags (for detection), optionally, one or more TAG stop codons and the phage protein pIII. Using various suppressor and non-suppressor strains of E. coli and with the addition of glucose, iso-propyl thio-β-D-galactoside (IPTG) or a helper phage, such as VCS M13, the vector is able to replicate as a plasmid with no expression, produce large quantities of the polypeptide library member only, or produce phage, some of which contain at least one copy of the polypeptide-pIII fusion on their surface.

An example of a preferred vector is the pHEN1 phagemid vector (Hoogenboom et al., 1991, Nucl. Acids Res. 19: 4133-4137; sequence is available, e.g., as SEQ ID NO: 7 in WO 03/031611), in which the production of pIII fusion protein is under the control of the LacZ promoter, which is inhibited in the presence of glucose and induced with IPTG. When grown in suppressor strains of E. coli, e.g., TG1, the gene III fusion protein is produced and packaged into phage, while growth in non-suppressor strains, e.g., HB2151, permits the secretion of soluble fusion protein into the bacterial periplasm and into the culture medium. Because the expression of gene III prevents later infection with helper phage, the bacteria harboring the phagemid vectors are propagated in the presence of glucose before infection with VCSM13 helper phage for phage rescue.

Construction of vectors according to the invention employs conventional ligation techniques. Isolated vectors or DNA fragments are cleaved, tailored, and re-ligated in the form desired to generate the required vector. If desired, sequence analysis to confirm that the correct sequences are present in the constructed vector is performed using standard methods. Suitable methods for constructing expression vectors, preparing in vitro transcripts, introducing DNA into host cells, and performing analyses for assessing expression and function are known to those skilled in the art. The presence of a gene sequence in a sample is detected, or its amplification and/or expression quantified by conventional methods, such as Southern or Northern analysis, Western blotting, dot blotting of DNA, RNA or protein, in situ hybridization, immunocytochemistry or sequence analysis of nucleic acid or protein molecules. Those skilled in the art will readily envisage how these methods may be modified, if desired.

PCR Mutagenesis:

The primer is complementary to a portion of a target molecule present in a pool of nucleic acid molecules used in the preparation of sets of nucleic acid repertoire members encoding polypeptide repertoire members. Most often, primers are prepared by synthetic methods, either chemical or enzymatic. Mutagenic oligonucleotide primers are generally 15 to 100 nucleotides in length, ideally from 20 to 40 nucleotides, although oligonucleotides of different length are of use.

Typically, selective hybridization occurs when two nucleic acid sequences are substantially complementary (at least about 65% complementary over a stretch of at least 14 to 25 nucleotides, preferably at least about 75%, more preferably at least about 85% or 90% complementary). See Kanehisa, 1984, Nucleic Acids Res. 12: 203, incorporated herein by reference. As a result, it is expected that a certain degree of mismatch at the priming site is tolerated. Such mismatch may be small, such as a mono-, di- or tri-nucleotide. Alternatively, it may comprise nucleotide loops, which are defined herein as regions in which mismatch encompasses an uninterrupted series of four or more nucleotides.

Overall, five factors influence the efficiency and selectivity of hybridization of the primer to a second nucleic acid molecule. These factors, which are (i) primer length, (ii) the nucleotide sequence and/or composition, (iii) hybridization temperature, (iv) buffer chemistry and (v) the potential for steric hindrance in the region to which the primer is required to hybridize, are important considerations when non-random priming sequences are designed.

There is a positive correlation between primer length and both the efficiency and accuracy with which a primer will anneal to a target sequence; longer sequences have a higher melting temperature (T_{M}) than do shorter ones, and are less likely to be repeated within a given target sequence, thereby minimizing promiscuous hybridization. Primer sequences with a high G-C content or that comprise palindromic sequences tend to self-hybridize, as do their intended target sites, since unimolecular, rather than bimolecular, hybridization kinetics are generally favored in solution; at the same time, it is important to design a primer containing sufficient numbers of G-C nucleotide pairings to bind the target sequence tightly, since each such pair is bound by three hydrogen bonds, rather than the two that are found when A and T bases pair. Hybridization temperature varies inversely with primer annealing efficiency, as does the concentration of organic solvents, e.g. formamide, that might be included in a hybridization mixture, while increases in salt concentration facilitate binding. Under stringent hybridization conditions, longer probes hybridize more efficiently than do shorter ones, which are sufficient under more permissive conditions. Stringent hybridization conditions for primers typically include salt concentrations of less than about 1M, more usually less than about 500 mM and preferably less than about 200 mM. Hybridization temperatures range from as low as 0° C. to greater than 22° C., greater than about 30° C., and (most often) in excess of about 37° C. Longer fragments may require higher hybridization temperatures for specific hybridization. As several factors affect the stringency of hybridization, the combination of parameters is more important than the absolute measure of any one alone.

Primers are designed with these considerations in mind. While estimates of the relative merits of numerous sequences may be made mentally by one of skill in the art, computer programs have been designed to assist in the evaluation of these several parameters and the optimization of primer sequences. Examples of such programs are “PrimerSelect” of the DNAStar™ software package (DNAStar, Inc.; Madison, Wis.) and OLIGO 4.0 (National Biosciences, Inc.). Once designed, suitable oligonucleotides are prepared by a suitable method, e.g. the phosphoramidite method described by Beaucage and Carruthers, 1981, Tetrahedron Lett. 22: 1859) or the triester method according to Matteucci and Caruthers, 1981, J. Am. Chem. Soc. 103: 3185, both incorporated herein by reference, or by other chemical methods using either a commercial automated oligonucleotide synthesizer or, for example, VLSIPS™ technology.

PCR is performed using template DNA (at least 1 fg; more usefully, 1-1000 ng) and at least 25 pmol of oligonucleotide primers; it may be advantageous to use a larger amount of primer when the primer pool is heavily heterogeneous, as each sequence is represented by only a small fraction of the molecules of the pool, and amounts become limiting in the later amplification cycles. A typical reaction mixture includes: 2 μl of DNA, 25 pmol of oligonucleotide primer, 2.5 μl of 10×PCR buffer 1 (Perkin-Elmer), 0.4 μl of 1.25 μM dNTP, 0.15 μl (or 2.5 units) of Taq DNA polymerase (Perkin Elmer) and deionized water to a total volume of 25 μl. Mineral oil is overlaid and the PCR is performed using a programmable thermal cycler.

The length and temperature of each step of a PCR cycle, as well as the number of cycles, is adjusted in accordance to the stringency requirements in effect. Annealing temperature and timing are determined both by the efficiency with which a primer is expected to anneal to a template and the degree of mismatch that is to be tolerated; obviously, when nucleic acid molecules are simultaneously amplified and mutagenized, mismatch is required, at least in the first round of synthesis. In attempting to amplify a population of molecules using a mixed pool of mutagenic primers, the loss, under stringent (high-temperature) annealing conditions, of potential mutant products that would only result from low melting temperatures is weighed against the promiscuous annealing of primers to sequences other than the target site. The ability to optimize the stringency of primer annealing conditions is well within the knowledge of one of skill in the art. An annealing temperature of between 30° C. and 72° C. is used. Initial denaturation of the template molecules normally occurs at between 92° C. and 99° C. for 4 minutes, followed by 20-40 cycles consisting of denaturation (94-99° C. for 15 seconds to 1 minute), annealing (temperature determined as discussed above; 1-2 minutes), and extension (72° C. for 1-5 minutes, depending on the length of the amplified product). Final extension is generally for 4 minutes at 72° C., and may be followed by an indefinite (0-24 hour) step at 4° C.

Screening Single Immunoglobulin Variable Domains for Antigen Binding:

Following expression of a repertoire of single immunoglobulin variable domains on the surface of phage, selection is performed by contacting the phage repertoire with immobilized target antigen, washing to remove unbound phage, and propagation of the bound phage, the whole process frequently referred to as “panning.” Alternatively, phage are pre-selected for the expression of properly folded member variants by panning against an immobilized generic ligand (e.g., protein A or protein L) that is only bound by folded members. This has the advantage of reducing the proportion of non-functional members, thereby increasing the proportion of members likely to bind a target antigen. Pre-selection with generic ligands is taught in WO 99/20749. The screening of phage antibody libraries is generally described, for example, by Harrison et al., 1996, Meth. Enzymol. 267: 83-109.

Screening is commonly performed using purified antigen immobilized on a solid support, for example, plastic tubes or wells, or on a chromatography matrix, for example Sepharose™ (Pharmacia). Screening or selection can also be performed on complex antigens, such as the surface of cells (Marks et al., 1993, BioTechnology 11: 1145; de Kruif et al., 1995, Proc. Natl. Acad. Sci. U.S.A. 92: 3938). Another alternative involves selection by binding biotinylated antigen in solution, followed by capture on streptavidin-coated beads.

In a preferred aspect, panning is performed by immobilizing antigen (generic or specific) on tubes or wells in a plate, e.g., Nunc MAXISORP™ immunotube 8 well strips. Wells are coated with 150 μl of antigen (100 μg/ml in PBS) and incubated overnight. The wells are then washed 3 times with PBS and blocked with 400 μl PBS-2% skim milk (2% MPBS) at 37° C. for 2 hr. The wells are rinsed 3 times with PBS and phage are added in 2% MPBS. The mixture is incubated at room temperature for 90 minutes and the liquid, containing unbound phage, is removed. Wells are rinsed 10 times with PBS-0.1% tween 20, and then 10 times with PBS to remove detergent. Bound phage are eluted by adding 200 μl of freshly prepared 100 mM triethylamine, mixing well and incubating for 10 min at room temperature. Eluted phage are transferred to a tube containing 100 μl of 1M Tris-HCl, pH 7.4 and vortexed to neutralize the triethylamine. Exponentially-growing E. coli host cells (e.g., TG1) are infected with, for example, 150 ml of the eluted phage by incubating for 30 min at 37° C. Infected cells are spun down, resuspended in fresh medium and plated in top agarose. Phage plaques are eluted or picked into fresh cultures of host cells to propagate for analysis or for further rounds of selection. One or more rounds of plaque purification are performed if necessary to ensure pure populations of selected phage. Other screening approaches are described by Harrison et al., 1996, supra.

Following identification of phage expressing a single immunoglobulin variable domain that binds a desired target, if a phagemid vector such as pHEN1 has been used, the variable domain fusion protein are easily produced in soluble form by infecting non-suppressor strains of bacteria, e.g., HB2151 that permit the secretion of soluble gene III fusion protein. Alternatively, the V domain sequence can be sub-cloned into an appropriate expression vector to produce soluble protein according to methods known in the art.

Purification and Concentration of Single Immunoglobulin Variable Domains:

Single immunoglobulin variable domain polypeptides secreted into the periplasmic space or into the medium of bacteria are harvested and purified according to known methods (Harrison et al., 1996, supra). Skerra & Pluckthun (1988, Science 240: 1038) and Breitling et al. (1991, Gene 104: 147) describe the harvest of antibody polypeptides from the periplasm, and Better et al. (1988, Science 240: 1041) describes harvest from the culture supernatant. Purification can also be achieved by binding to generic ligands, such as protein A or Protein L. Alternatively, the variable domains can be expressed with a peptide tag, e.g., the Myc, HA or 6×-His tags, which facilitates purification by affinity chromatography.

Polypeptides are concentrated by several methods well known in the art, including, for example, ultrafiltration, diafiltration and tangential flow filtration. The process of ultrafiltration uses semi-permeable membranes and pressure to separate molecular species on the basis of size and shape. The pressure is provided by gas pressure or by centrifugation. Commercial ultrafiltration products are widely available, e.g., from Millipore (Bedford, Mass.; examples include the Centricon™ and Microcon™ concentrators) and Vivascience (Hannover, Germany; examples include the Vivaspin™ concentrators). By selection of a molecular weight cutoff smaller than the target polypeptide (usually ⅓ to ⅙ the molecular weight of the target polypeptide, although differences of as little as 10 kD can be used successfully), the polypeptide is retained when solvent and smaller solutes pass through the membrane. Thus, a molecular weight cutoff of about 5 kD is useful for concentration of single immunoglobulin variable domain polypeptides described herein.

Diafiltration, which uses ultrafiltration membranes with a “washing” process, is used where it is desired to remove or exchange the salt or buffer in a polypeptide preparation. The polypeptide is concentrated by the passage of solvent and small solutes through the membrane, and remaining salts or buffer are removed by dilution of the retained polypeptide with a new buffer or salt solution or water, as desired, accompanied by continued ultrafiltration. In continuous diafiltration, new buffer is added at the same rate that filtrate passes through the membrane. A diafiltration volume is the volume of polypeptide solution prior to the start of diafiltration—using continuous diafiltration, greater than 99.5% of a fully permeable solute can be removed by washing through six diafiltration volumes with the new buffer. Alternatively, the process can be performed in a discontinuous manner, wherein the sample is repeatedly diluted and then filtered back to its original volume to remove or exchange salt or buffer and ultimately concentrate the polypeptide. Equipment for diafiltration and detailed methodologies for its use are available, for example, from Pall Life Sciences (Ann Arbor, Mich.) and Sartorius AG/Vivascience (Hannover, Germany).

Tangential flow filtration (TFF), also known as “cross-flow filtration,” also uses ultrafiltration membrane. Fluid containing the target polypeptide is pumped tangentially along the surface of the membrane. The pressure causes a portion of the fluid to pass through the membrane while the target polypeptide is retained above the filter. In contrast to standard ultrafiltration, however, the retained molecules do not accumulate on the surface of the membrane, but are carried along by the tangential flow. The solution that does not pass through the filter (containing the target polypeptide) can be repeatedly circulated across the membrane to achieve the desired degree of concentration. Equipment for TFF and detailed methodologies for its use are available, for example, from Millipore (e.g., the ProFlux M12™ Benchtop TFF system and the Pellicon™ systems), Pall Life Sciences (e.g., the Minim™ Tangential Flow Filtration system).

Protein concentration is measured in a number of ways that are well known in the art. These include, for example, amino acid analysis, absorbance at 280 nm, the “Bradford” and “Lowry” methods, and SDS-PAGE. The most accurate method is total hydrolysis followed by amino acid analysis by HPLC, concentration is then determined then comparison with the known sequence of the single immunoglobulin variable domain polypeptide. While this method is the most accurate, it is expensive and time-consuming. Protein determination by measurement of UV absorbance at 280 nm faster and much less expensive, yet relatively accurate and is preferred as a compromise over amino acid analysis. Absorbance at 280 nm was used to determine protein concentrations reported in the Examples described herein.

“Bradford” and “Lowry” protein assays (Bradford, 1976, Anal. Biochem. 72: 248-254; Lowry et al., 1951, J. Biol. Chem. 193: 265-275) compare sample protein concentration to a standard curve most often based on bovine serum albumin (BSA). These methods are less accurate, tending to underestimate the concentration of single immunoglobulin variable domains. Their accuracy could be improved, however, by using a V_{H} or V_{κ} single domain polypeptide as a standard.

An additional protein assay method is the bicinchoninic acid assay described in U.S. Pat. No. 4,839,295 (incorporated herein by reference) and marketed by Pierce Biotechnology (Rockford, Ill.) as the “BCA Protein Assay” (e.g., Pierce Catalog No. 23227).

The SDS-PAGE method uses gel electrophoresis and Coomassie Blue staining in comparison to known concentration standards, e.g., known amounts of a single immunoglobulin variable domain polypeptide. Quantitation can be done by eye or by densitometry.

Single immunoglobulin variable domain antigen-binding polypeptides described herein retain solubility at high concentration (e.g., at least 4.8 mg (˜400 μM) in aqueous solution (e.g., PBS), and preferably at least 5 mg/ml (˜417 μM), 10 mg/ml (˜833 μM), 20 mg/ml (˜1.7 mM), 25 mg/ml (˜2.1 mM), 30 mg/ml (˜2.5 mM), 35 mg/ml (˜2.9 mM), 40 mg/ml (˜3.3 mM), 45 mg/ml (˜3.75 mM), 50 mg/ml (˜4.2 mM), 55 mg/ml (˜4.6 mM) 60 mg/ml (˜5.0 mM), 65 mg/ml (˜5.4 mM), 70 mg/ml (˜5.8 mM), 75 mg/ml (˜6.3 mM), 100 mg/ml (˜8.33 mM), 150 mg/ml (˜12.5 mM), 200 mg/ml (˜16.7 mM), 240 mg/ml (˜20 mM) or higher). One structural feature that promotes high solubility is the relatively small size of the single immunoglobulin variable domain polypeptides. A full length conventional four chain antibody, e.g., IgG is about 150 kD in size. In contrast, single immunoglobulin variable domains, which all have a general structure comprising 4 framework (FW) regions and 3 CDRs, have a size of approximately 12 kD, or less than 1/10 the size of a conventional antibody. Similarly, single immunoglobulin variable domains are approximately ½ the size of an scFv molecule (˜26 kD), and approximately ⅕ the size of a Fab molecule (˜60 kD). It is preferred that the size of a single immunoglobulin variable domain-containing structure disclosed herein is 100 kD or less, including structures of, for example, about 90 kD or less, 80 kD or less, 70 kD or less, 60 kD or less, 50 kD or less, 40 kD or less, 30 kD or less, 20 kD or less, down to and including about 12 kD, or a single immunoglobulin variable domain in isolation.

The solubility of a polypeptide is primarily determined by the interactions of the amino acid side chains with the surrounding solvent. Hydrophobic side chains tend to be localized internally as a polypeptide folds, away from the solvent-interacting surfaces of the polypeptide. Conversely, hydrophilic residues tend to be localized at the solvent-interacting surfaces of a polypeptide. Generally, polypeptides having a primary sequence that permits the molecule to fold to expose more hydrophilic residues to the aqueous environment are more soluble than one that folds to expose fewer hydrophilic residues to the surface. Thus, the arrangement and number of hydrophobic and hydrophilic residues is an important determinant of solubility. Other parameters that determine polypeptide solubility include solvent pH, temperature, and ionic strength. In a common practice, the solubility of polypeptides can be maintained or enhanced by the addition of glycerol (e.g., ˜10% v/v) to the solution.

As discussed above, specific amino acid residues have been identified in conserved residues of human V_{H} domains that vary in the V_{H} domains of camelid species, which are generally more soluble than human V_{H} domains. These include, for example, Gly 44 (Glu in camelids), Leu 45 (Arg in camelids) and Trp 47 (Gly in camelids). Amino acid residue 103 of V_{H} is also implicated in solubility, with mutation from Trp to Arg tending to confer increased V_{H} solubility.

In preferred aspects of the invention, single immunoglobulin variable domain polypeptides are based on the DP47 germline V_{H} gene segment or the DPK9 germline V_{κ} gene segment. Examples of single immunoglobulin variable domain polypeptides based on these germline gene segments that have high solubility are provided herein. Thus, these germline gene segments are capable, particularly when diversified at selected structural locations described herein, of producing specific binding single immunoglobulin variable domain polypeptides that are highly soluble. In particular, the four framework regions, which are preferably not diversified, can contribute to the high solubility of the resulting proteins.

It is expected that a single immunoglobulin variable domain that is highly homologous to one having a known high solubility will also tend to be highly soluble. Thus, as one means of prediction or recognition that a given single immunoglobulin variable domain would have the high solubility recited herein, one can compare the sequence of a single immunoglobulin variable domain polypeptide to one or more single immunoglobulin variable domain polypeptides having known solubility. Thus, when a single immunoglobulin variable domain polypeptide is identified that has high binding affinity but unknown solubility, comparison of its amino acid sequence with that of one or more (preferably more) single immunoglobulin variable domain polypeptides known to have high solubility (e.g., a dAb sequence disclosed herein) can permit prediction of its solubility. While it is not an absolute predictor, where there is a high degree of similarity to a known highly soluble sequence, e.g., 90-95% or greater similarity, and particularly where there is a high degree of similarity with respect to hydrophilic amino acid residues, or residues likely to be exposed at the solvent interface, it is more likely that a newly identified binding polypeptide will have solubility similar to that of the known highly soluble sequence.

Molecular modeling software can also be used to predict the solubility of a polypeptide sequence relative to that of a polypeptide of known solubility. For example, the substitution or addition of a hydrophobic residue at the solvent-exposed surface, relative to a molecule of known solubility that has a less hydrophobic or even hydrophilic residue exposed in that position is expected to decrease the relative solubility of the polypeptide. Similarly, the substitution or addition of a more hydrophilic residue at such a location is expected to increase the relative solubility. That is, a change in the net number of hydrophilic or hydrophobic residues located at the surface of the molecule (or the overall hydrophobic or hydrophilic nature of the surface-exposed residues) relative to a single immunoglobulin variable domain polypeptide structure with known solubility can predict the relative solubility of a single immunoglobulin variable domain polypeptide.

Alternatively, or in conjunction with such prediction, one can determine limits of a single immunoglobulin variable domain polypeptide's solubility by simply concentrating the polypeptide.

Affinity Determination:

Isolated single immunoglobulin variable domain-containing polypeptides as described herein have affinities (dissociation constant, K_{d},=K_{off}/Kₒₙ) of at least 300 nM or less, and preferably at least 300 nM-50 pM, 200 nM-50 pM, and more preferably at least 100 nM-50 pM, 75 nM-50 pM, 50 nM-50 pM, 25 nM-50 pM, 10 nM-50 pM, 5 nM-50 pM, 1 nM-50 pM, 950 pM-50 pM, 900 pM-50 pM, 850 pM-50 pM, 800 pM-50 pM, 750 pM-50 pM, 700 pM-50 pM, 650 pM-50 pM, 600 pM-50 pM, 550 pM-50 pM, 500 pM-50 pM, 450 pM-50 pM, 400 pM-50 pM, 350 pM-50 pM, 300 pM-50 pM, 250 pM-50 pM, 200 pM-50 pM, 150 pM-50 pM, 100 pM-50 pM, 90 pM-50 pM, 80 pM-50 pM, 70 pM-50 pM, 60 pM-50 pM, or even as low as 50 pM.

The antigen-binding affinity of a variable domain polypeptide can be conveniently measured by SPR using the BIAcore system (Pharmacia Biosensor, Piscataway, N.J.). In this method, antigen is coupled to the BIAcore chip at known concentrations, and variable domain polypeptides are introduced. Specific binding between the variable domain polypeptide and the immobilized antigen results in increased protein concentration on the chip matrix and a change in the SPR signal. Changes in SPR signal are recorded as resonance units (RU) and displayed with respect to time along the Y axis of a sensorgram. Baseline signal is taken with solvent alone (e.g., PBS) passing over the chip. The net difference between baseline signal and signal after completion of variable domain polypeptide injection represents the binding value of a given sample. To determine the off rate (K_{off}), on rate (Kₒₙ) and dissociation rate (K_{d}) constants, BIAcore kinetic evaluation software (e.g., version 2.1) is used.

High affinity is dependent upon the complementarity between a surface of the antigen and the CDRs of the antibody or antibody fragment. Complementarity is determined by the type and strength of the molecular interactions possible between portions of the target and the CDR, for example, the potential ionic interactions, van der Waals attractions, hydrogen bonding or other interactions that can occur. CDR3 tends to contribute more to antigen binding interactions than CDRs 1 and 2, probably due to its generally larger size, which provides more opportunity for favorable surface interactions. (See, e.g., Padlan et al., 1994, Mol. Immunol. 31: 169-217; Chothia & Lesk, 1987, J. Mol. Biol. 196: 904-917; and Chothia et al., 1985, J. Mol. Biol. 186: 651-663.) High affinity indicates single immunoglobulin variable domain/antigen pairings that have a high degree of complementarity, which is directly related to the structures of the variable domain and the target.

The structures conferring high affinity of a single immunoglobulin variable domain polypeptide for a given antigen can be highlighted using molecular modeling software that permits the docking of an antigen with the polypeptide structure. Generally, a computer model of the structure of a single immunoglobulin variable domain of known affinity can be docked with a computer model of a polypeptide or other target antigen of known structure to determine the interaction surfaces. Given the structure of the interaction surfaces for such a known interaction, one can then predict the impact, positive or negative, of conservative or less-conservative substitutions in the variable domain sequence on the strength of the interaction, thereby permitting the rational design of improved binding molecules.

Multimeric Forms of Single Immunoglobulin Variable Domains:

In one aspect, a single immunoglobulin variable domain as described herein is multimerized, as for example, homodimers, homotrimers or higher order homomultimers. Multimerization can increase the strength of antigen binding through the avidity effect, wherein the strength of binding is related to the sum of the binding affinities of the multiple binding sites.

Homomultimers are prepared through expression of single immunoglobulin variable domains fused, for example, through a peptide linker, leading to the configuration dAb-linker-dAb or a higher multiple of that arrangement. The homomultimers can also be linked to additional moieties, e.g., a polypeptide sequence that increases serum half-life or another effector moiety, e.g., a toxin or targeting moiety. Any linker peptide sequence can be used to generate homomultimers, e.g., a linker sequence as would be used in the art to generate an scFv. One commonly useful linker comprises repeats of the peptide sequence (Gly₄Ser)ₙ, wherein n=1 to about 10. For example, the linker can be (Gly₄Ser)₃, (Gly₄Ser)₅, (Gly₄Ser)₇ or another multiple of the (Gly₄Ser) sequence.

An alternative to the expression of multimers as monomers linked by peptide sequences is linkage of the monomeric single immunoglobulin variable domains post-translationally through, for example, disulfide bonding or other chemical linkage. For example, a free cysteine is engineered, e.g., at the C-terminus of the monomeric polypeptide, permits disulfide bonding between monomers. In this aspect or others requiring a free cysteine, the cysteine is introduced by including a cysteine codon (TGT, TGC) into a PCR primer adjacent to the last codon of the dAb sequence (for a C-terminal cysteine, the sequence in the primer will actually be the reverse complement, i.e., ACA or GCA, because it will be incorporated into the downstream PCR primer) and immediately before one or more stop codons. If desired, a linker peptide sequence, e.g., (Gly₄Ser)ₙ is placed between the dAb sequence and the free cysteine. Expression of the monomers having a free cysteine residue results in a mixture of monomeric and dimeric forms in approximately a 1:1 mixture. Dimers are separated from monomers using gel chromatography, e.g., ion-exchange chromatography with salt gradient elution.

Alternatively, an engineered free cysteine is used to couple monomers through thiol linkages to a multivalent chemical linker, such as a trimeric maleimide molecule (e.g., Tris[2-maleimidoethyl]amine, TMEA) or a bi-maleimide PEG molecule (available from, for example, Nektar (Shearwater).

Target Antigens

Target antigens for single immunoglobulin variable domain polypeptides as described herein are polypeptide antigens, preferably human polypeptide antigens related to a disease or disorder. That is, target antigens as described herein are therapeutically relevant targets. A “therapeutically relevant target” is one which, when bound by a single immunoglobulin variable domain or other antibody polypeptide that binds target antigen and acts as an antagonist or agonist of that target's activity, has a beneficial effect on the human individual in which the target is bound. A “beneficial effect” is demonstrated by at least a 10% improvement in one or more clinical indicia of a disease or disorder, or, alternatively, where a prophylactic use of the single immunoglobulin variable domain polypeptide is desired, by an increase of at least 10% in the time before symptoms of the targeted disease or disorder are observed, relative to an individual not treated with the single immunoglobulin variable domain polypeptide preparation. Non-limiting examples of antigens that are suitable targets for single immunoglobulin variable domain polypeptides as described herein include cytokines, cytokine receptors, enzymes, enzyme co-factors, or DNA binding proteins. Suitable cytokines and growth factors include but are not limited to: ApoE, Apo-SAA, BDNF, Cardiotrophin-1, EGF, EGF receptor, ENA-78, Eotaxin, Eotaxin-2, Exodus-2, FGF-acidic, FGF-basic, fibroblast growth factor-10, FLT3 ligand, Fractalkine (CX3C), GDNF, G-CSF, GM-CSF, GF-β1, insulin, IFN-g, IGF-I, IGF-II, IL-1α, IL-1β, IL-2, IL-3, IL-4, IL-5, IL-6, IL-7, IL-8 (72 a.a.), IL-8 (77 a.a.), IL-9, IL-10, IL-11, IL-12, IL-13, IL-15, IL-16, IL-17, IL-18 (IGIF), Inhibin α, Inhibin β, IP-10, keratinocyte growth factor-2 (KGF-2), KGF, Leptin, LIF, Lymphotactin, Mullerian inhibitory substance, monocyte colony inhibitory factor, monocyte attractant protein, M-CSF, MDC (67 a.a.), MDC (69 a.a.), MCP-1 (MCAF), MCP-2, MCP-3, MCP-4, MDC (67 a.a.), MDC (69 a.a.), MIG, MIP-1α, MIP-1β, MIP-3α, MIP-3β, MIP-4, myeloid progenitor inhibitor factor-1 (MPIF-1), NAP-2, Neurturin, Nerve growth factor, β-NGF, NT-3, NT-4, Oncostatin M, PDGF-AA, PDGF-AB, PDGF-BB, PF-4, RANTES, SDF1α, SDF1β, SCF, SCGF, stem cell factor (SCF), TARC, TACE recognition site, TGF-α, TGF-β, TGF-β 2, TGF-β 3, tumor necrosis factor (TNF), TNF-α, TNF-β, TNF receptor I (p55), TNF receptor II, TNIL-1, TPO, VEGF, VEGF receptor 1, VEGF receptor 2, VEGF receptor 3, GCP-2, GRO/MGSA, GRO-β, GRO-γ, HCC1, 1-309, HER 1, HER 2, HER 3 and HER 4. Cytokine receptors include receptors for each of the foregoing cytokines, e.g., IL-1R, IL-6R, IL-10R, IL-18R, etc. It will be appreciated that this list is by no means exhaustive.

In one aspect, a single immunoglobulin variable domain is linked to another single immunoglobulin variable domain to form a homodimer or heterodimer in which each individual domain is capable of binding its cognate antigen. Fusing single immunoglobulin variable domains as homodimers can increase the efficiency of target binding. e.g., through the avidity effect. Fusing single immunoglobulin variable domains as heterodimers, wherein each monomer binds a different target antigen, can produce a dual-specific ligand capable, for example, of bridging the respective target antigens. Such dual specific ligands may be used to target cytokines and other molecules which cooperate synergistically in therapeutic situations in the body of an organism. Thus, there is provided a method for synergising the activity of two or more cytokines, comprising administering a dual specific single immunoglobulin variable domain heterodimer capable of binding to the two or more cytokines. In this aspect, the dual specific ligand may be any dual specific ligand, including a ligand composed of complementary and/or non-complementary domains. For example, this aspect relates to combinations of V_{H} domains and V_{L} domains, V_{H} domains only and V_{L} domains only.

Preferably, the cytokines bound by the dual specific single immunoglobulin variable domain heterodimer of this aspect of the invention are selected from the following list:

| Pairing | Evidence for therapeutic impact |
|---|---|
| TNF/TGF-β | TGF-β and TNF when injected into the |
| | ankle joint of mouse collagen induced |
| | arthritis model significantly enhanced joint |
| | inflammation. In non-collagen challenged |
| | mice there was no effect. |
| TNF/IL-1 | TNF and IL-1 synergize in the pathology of |
| | uveitis. |
| | TNF and IL-1 synergize in the pathology of |
| | malaria (hypoglycaemia, NO). |
| | TNF and IL-1 synergize in the induction of |
| | polymorphonuclear (PMN) cells migration |
| | in inflammation. |
| | IL-1 and TNF synergize to induce PMN |
| | infiltration into the mouse peritoneum. |
| | IL-1 and TNF synergize to induce the |
| | secretion of IL-1 by endothelial cells. |
| | Important in inflammation. |
| | IL-1 or TNF alone induced some cellular |
| | infiltration into rabbit knee synovium. IL-1 |
| | induced PMNs, TNF - monocytes. |
| | Together they induced a more severe |
| | infiltration due to increased PMNs. |
| | Circulating myocardial depressant |
| | substance (present in sepsis) is low levels |
| | of IL-1 and TNF acting synergistically. |
| TNF/IL-2 | References relating to synergisitic |
| | activation of killer T-cells. |
| TNF/IL-3 | |
| TNF/IL-4 | IL-4 and TNF synergize to induce VCAM |
| | expression on endothelial cells. Implied to |
| | have a role in asthma. Same for synovium - |
| | implicated in RA. |
| | TNF and IL-4 synergize to induce IL-6 |
| | expression in keratinocytes. |
| TNF/IL-6 | |
| TNF/IL-8 | TNF and IL-8 synergized with PMNs to |
| | activate platelets. Implicated in Acute |
| | Respiratory Distress Syndrome. |
| TNF/IL-10 | IL-10 induces and synergizes with TNF in |
| | the induction of HIV expression in |
| | chronically infected T-cells. |
| TNF/IL-12 | |
| TNF/IFN-γ | MHC induction in the brain. |
| | Synergize in anti-viral response/IFN-b |
| | induction. |
| | Neutrophil activation/respiratory burst. |
| | Endothelial cell activation |
| | Toxicities noted when patients treated with |
| | TNF/IFN-γ as anti-viral therapy (will find |
| | out more). |
| | Fractalkine expression by human |
| | astrocytes. |
| | Many papers on inflammatory responses - |
| | i.e. LPS, also macrophage activation. |
| | Anti-TNF and anti-IFN-γ synergize to |
| | protect mice from lethal endotoxemia. |
| TGF-β/IL-1 | Prostaglndin synthesis by osteoblasts |
| | IL-6 production by intestinal epithelial cells |
| | (inflammation model) |
| | Stimulates IL-11 and IL-6 in lung fibroblasts |
| | (inflammation model) |
| | IL-6 and IL-8 production in the retina |
| TGF-β/IL-6 | Chondrocarcoma proliferation |
| IL-1/IL-2 | B-cell activation |
| | LAK cell activation |
| | T-cell activation |
| IL-1/IL-3 | |
| IL-1/IL-4 | B-cell activation |
| | IL-4 induces IL-1 expression in endothelial |
| | cell activation. |
| IL-1/IL-6 | B cell activation |
| | T cell activation (can replace accessory |
| | cells) |
| | IL-1 induces IL-6 expression |
| | C3 and serum amyloid expression (acute |
| | phase response) |
| | HIV expression |
| | Cartilage collagen breakdown. |
| IL-1/IL-8 | |
| IL-1/IL-10 | |
| IL-1/IFN-g | |
| IL-2/IL-3 | T-cell proliferation |
| | B cell proliferation |
| IL-2/IL-4 | B-cell proliferation |
| | T-cell proliferation |
| IL-2/IL-5 | B-cell proliferation/Ig secretion |
| | IL-5 induces IL-2 receptors on B-cells |
| IL-2/IL-6 | Development of cytotoxic T-cells |
| IL-2/IL-7 | |
| IL-2/IL-10 | B-cell activation |
| IL-2/IL-12 | |
| IL-2/IL-15 | |
| IL-2/IFN-γ | Ig secretion by B-cells |
| | IL-2 induces IFN-g expression by T-cells |
| IL-2/IFN-α/β | |
| IL-3/IL-4 | Synergize in mast cell growth |
| IL-3/IL-5 | |
| IL-3/IL-6 | |
| IL-3/IFN-γ | |
| IL-4/IL-5 | Enhanced mast cell histamine etc. secretion |
| | in response to IgE |
| IL-4/IL-6 | |
| IL-4/IL-10 | |
| IL-4/IL-12 | |
| IL-4/IL-13 | |
| IL-4/IFN-γ | |
| IL-4/SCF | Mast cell proliferation |
| IL-5/IL-6 | |
| IL-5/IFN-γ | |
| IL-6/IL-10 | |
| IL-6/IL-11 | |
| IL-6/IFN-γ | |
| IL-10/IL-12 | |
| IL-10/IFN-γ | |
| IL-12/IL-18 | |
| IL-12/IFN-γ | IL-12 induces IFN-g expression by B and |
| | T-cells as part of immune stimulation. |
| IL-18/IFN-γ | |
| Anti-TNF/anti- | Synergistic therapeutic effect in DBA/1 |
| CD4 | arthritic mice. |

The amino acid and nucleotide sequences for the target antigens listed above and others are known and available to those of skill in the art. Standard methods of recombinant protein expression are used by one of skill in the art to express and purify these and other antigens where necessary, e.g., to pan for single immunoglobulin variable domains that bind the target antigen.

Functional Assays

Single immunoglobulin variable domains as described herein have neutralizing activity (e.g., antagonizing activity) or agonizing activity towards their target antigens. The activity (whether neutralizing or agonizing) of a single immunoglobulin variable domain polypeptide as described herein is measured relative to the activity of the target antigen in the absence of the polypeptide in any accepted assay for such activity. For example, if the target antigen is an enzyme, an in vivo or in vitro functional assay that monitors the activity of that enzyme is used to monitor the activity or effect of a single immunoglobulin variable domain polypeptide.

Where, for example, the target antigen is a receptor, e.g., a cytokine receptor, activity is measured in terms of reduced or increased ligand binding to the receptor or in terms of reduced or increased signaling activity by the receptor in the presence of the single immunoglobulin variable domain polypeptide. Receptor signaling activity is measured by monitoring, for example, receptor conformation, co-factor or partner polypeptide binding, GDP for GTP exchange, a kinase, phosphatase or other enzymatic activity possessed by the activated receptor, or by monitoring a downstream result of such activity, such as expression of a gene (including a reporter gene) or other effect, including, for example, cell death, DNA replication, cell adhesion, or secretion of one or more molecules normally occurring as a result of receptor activation.

Where the target antigen is, for example, a cytokine or growth factor, activity is monitored by assaying binding of the cytokine to its receptor or by monitoring the activation of the receptor, e.g., by monitoring receptor signaling activity as discussed above. An example of a functional assay that measures a downstream effect of a cytokine is the L929 cell killing assay for TNF-α activity, which is well known in the art (see, for example, U.S. Pat. No. 6,090,382). The following L929 cytotoxicity assay is referred to herein as the “standard” L929 cytotoxicity assay. Anti-TNF single immunoglobulin variable domains (“anti-TNF dAbs”) are tested for the ability to neutralize the cytotoxic activity of TNF on mouse L929 fibroblasts (Evans, T. (2000) Molecular Biotechnology 15, 243-248). Briefly, L929 cells plated in microtiter plates are incubated overnight with anti-TNF dAbs, 100 pg/ml TNF and 1 mg/ml actinomycin D (Sigma, Poole, UK). Cell viability is measured by reading absorbance at 490 nm following an incubation with [3-(4,5-dimethylthiazol-2-yl)-5-(3-carboxymethoxyphenyl)-2-(4-sulfophenyl)-2H-tetrazolium (Promega, Madison, USA). Anti-TNF dAb activity leads to a decrease in TNF cytotoxicity and therefore an increase in absorbance compared with the TNF only control. A single immunoglobulin variable domain polypeptide described herein that is specific for TNF-α or TNF-α receptor has an IC₅₀ of 500 nM or less in this standard L929 cell assay, preferably 50 nM or less, 5 nM or less, 500 pM or less, 200 pM or less, 100 pM or less or even 50 pM.

Assays for the measurement of receptor binding by a ligand, e.g., a cytokine, are known in the art. As an example, anti-TNF dAbs can be tested for the ability to inhibit the binding of TNF to recombinant TNF receptor 1 (p55). Briefly, Maxisorp plates are incubated overnight with 30 mg/ml anti-human Fc mouse monoclonal antibody (Zymed, San Francisco, USA). The wells are washed with phosphate buffered saline (PBS) containing 0.05% Tween-20 and then blocked with 1% BSA in PBS before being incubated with 100 ng/ml TNF receptor 1 Fc fusion protein (R&D Systems, Minneapolis, USA). Anti-TNF dAb is mixed with TNF which is added to the washed wells at a final concentration of 10 ng/ml. TNF binding is detected with 0.2 mg/ml biotinylated anti-TNF antibody (HyCult biotechnology, Uben, Netherlands) followed by 1 in 500 dilution of horse radish peroxidase labelled streptavidin (Amersham Biosciences, UK) and incubation with TMB substrate (KPL, Gaithersburg, Md.). The reaction is stopped by the addition of HCl and the absorbance is read at 450 nm. Anti-TNF dAb inhibitory activity leads to a decrease in TNF binding and therefore to a decrease in absorbance compared with the TNF only control.

As an alternative when evaluating the effect of a single immunoglobulin variable domain polypeptide on the p55 TNF-α receptor, the following HeLa cell assay based on the induction of IL-8 secretion by TNF in HeLa cells can be used (method is adapted from that of Akeson, L. et al (1996) Journal of Biological Chemistry 271, 30517-30523, describing the induction of IL-8 by IL-1 in HUVEC; here we look at induction by human TNF alpha and we use HeLa cells instead of the HUVEC cell line). Briefly, HeLa cells plated in microtitre plates are incubated overnight with dAb and 300 pg/ml TNF. Following incubation, the supernatant is aspirated off the cells and the IL-8 concentration is measured via a sandwich ELISA (R&D Systems). Anti-TNFR1 dAb activity leads to a decrease in IL-8 secretion into the supernatant compared with the TNF only control.

Similar functional assays for the activity of other ligands (cytokines, growth factors, etc.) or their receptors are known to those of skill in the art and can be employed to evaluate the antagonistic or agonistic effect of single immunoglobulin variable domain polypeptides.

Increasing the In Vivo Half-Life of Single Immunoglobulin Variable Domain Polypeptides:

Increased half-life is useful in in vivo applications of immunoglobulins, especially antibodies and most especially antibody fragments of small size. Such fragments (Fvs, Fabs, scFvs, dAbs) suffer from rapid clearance from the body; thus, while they are able to reach most parts of the body rapidly, and are quick to produce and easier to handle, their in vivo applications have been limited by their only brief persistence in vivo.

In one aspect, a single immunoglobulin variable domain polypeptide as described herein is stabilized in vivo by fusion with a moiety that binds a protein or polypeptide antigen or epitope that can act to increase the in vivo half-life of the ligand. The protein or polypeptide antigen or epitope that can act to increase half-life is referred to herein as an “effector group.” One way to achieve stabilization of a single immunoglobulin variable domain polypeptide is to prepare a fusion of two or more single immunoglobulin variable domain polypeptides wherein at least one of the variable domain polypeptides binds an effector group and at least one of the remaining single immunoglobulin variable domain polypeptides in the fusion binds a therapeutically relevant target. Thus, the molecule of this aspect is at least a dual-specific ligand, comprising at least one single immunoglobulin variable domain specific for a therapeutically relevant target and at least one single immunoglobulin variable domain specific for a protein or polypeptide that increases the in vivo half-life of the ligand. The complex of such a dual-specific single immunoglobulin variable domain-containing polypeptide with the polypeptide effector group that increases half-life is referred to herein as a “dAb-effector group” composition. Examples of effector groups according to this aspect are described herein below.

Antigens or epitopes which increase the half-life of a ligand as described herein are advantageously present on proteins or polypeptides found in an organism in vivo. Examples, include extracellular matrix proteins, blood proteins, and proteins present in various tissues in the organism. The proteins act to reduce or prevent the rate of ligand clearance from the blood, for example by acting as bulking agents, or by anchoring the ligand to a desired site of action. Methods for pharmacokinetic analysis and determination of ligand half-life will be familiar to those skilled in the art. Details may be found in Kenneth, A et al: Chemical Stability of Pharmaceuticals: A Handbook for Pharmacists and in Peters et al, Pharmacokinetc analysis: A Practical Approach (1996). Reference is also made to “Pharmacokinetics”, M Gibaldi & D Perron, published by Marcel Dekker, 2 Rev. ex edition (1982), which describes pharmacokinetic parameters such as t alpha and t beta half lives and area under the curve (AUC).

Half lives (t1/2 alpha and t1/2 beta) and AUC can be determined from a curve of serum concentration of ligand against time. The WinNonlin analysis package (available from Pharsight Corp., Mountain View, Calif. 94040, USA) can be used, for example, to model the curve. In a first phase (the alpha phase) the ligand is undergoing mainly distribution in the patient, with some elimination. A second phase (beta phase) is the terminal phase when the ligand has been distributed and the serum concentration is decreasing as the ligand is cleared from the patient. The tα half life is the half life of the first phase and the tβ half life is the half life of the second phase. Thus, advantageously, the present invention provides a dAb-containing composition, e.g., a dAb-effector group composition, having a tα half-life in the range of 15 minutes or more. In one embodiment, the lower end of the range is 30 minutes, 45 minutes, 1 hour, 2 hours, 3 hours, 4 hours, 5 hours, 6 hours, 7 hours, 10 hours, 11 hours or 12 hours. In addition, or alternatively, a dAb-containing composition, e.g., a dAb-effector group composition, will have a tα half life in the range of up to and including 12 hours. In one embodiment, the upper end of the range is 11, 10, 9, 8, 7, 6 or 5 hours. An example of a suitable range is 1 to 6 hours, 2 to 5 hours or 3 to 4 hours.

Advantageously, the present invention provides a dAb containing composition, e.g. a dAb-effector group composition, comprising a ligand according to the invention having a tβ half-life in the range of 2.5 hours or more. In one embodiment, the lower end of the range is 3 hours, 4 hours, 5 hours, 6 hours, 7 hours, 10 hours, 11 hours, or 12 hours. In addition, or alternatively, a dAb containing composition, e.g. a dAb-effector group composition has a tβ half-life in the range of up to and including 21 days. In one embodiment, the upper end of the range is 12 hours, 24 hours, 2 days, 3 days, 5 days, 10 days, 15 days or 20 days. Advantageously a dAb containing composition according to the invention will have a tβ half life in the range 12 to 60 hours. In a further embodiment, it will be in the range 12 to 48 hours. In a further embodiment still, it will be in the range 12 to 26 hours.

In addition, or alternatively to the above criteria, the present invention provides a dAb containing composition comprising a ligand according to the invention having an AUC value (area under the curve) in the range of 1 mg.min/ml or more. In one embodiment, the lower end of the range is 5, 10, 15, 20, 30, 100, 200 or 300 mg.min/ml. In addition, or alternatively, a ligand or composition according to the invention has an AUC in the range of up to 600 mg.min/ml. In one embodiment, the upper end of the range is 500, 400, 300, 200, 150, 100, 75 or 50 mg.min/ml. Advantageously a ligand according to the invention will have an AUC in the range selected from the group consisting of the following: 15 to 150 mg.min/ml, 15 to 100 mg.min/ml, 15 to 75 mg.min/ml, and 15 to 50 mg.min/ml.

Antigens Capable of Increasing Ligand Half-Life

The dual specific ligands according to the invention are capable of binding to one or more molecules which can increase the half-life of the ligand in vivo. Typically, such molecules are polypeptides which occur naturally in vivo and which resist degradation or removal by endogenous mechanisms which remove unwanted material from the organism. For example, the molecule which increases the half-life of the organism may be selected from the following:

Proteins from the extracellular matrix; for example collagen, laminins, integrins and fibronectin. Collagens are the major proteins of the extracellular matrix. About 15 types of collagen molecules are currently known, found in different parts of the body, e.g. type I collagen (accounting for 90% of body collagen) found in bone, skin, tendon, ligaments, cornea, internal organs or type II collagen found in cartilage, invertebral disc, notochord, vitreous humour of the eye;

Proteins found in blood, including:

Plasma proteins such as fibrin, α-2 macroglobulin, serum albumin, fibrinogen A, fibrinogen B, serum amyloid protein A, heptaglobin, profilin, ubiquitin, uteroglobulin and β-2-microglobulin;

Enzymes and inhibitors such as plasminogen, lysozyme, cystatin C, alpha-1-antitrypsin and pancreatic trypsin inhibitor. Plasminogen is the inactive precursor of the trypsin-like serine protease plasmin. It is normally found circulating through the blood stream. When plasminogen becomes activated and is converted to plasmin, it unfolds a potent enzymatic domain that dissolves the fibrinogen fibers that entgangle the blood cells in a blood clot. This is called fibrinolysis;

Immune system proteins, such as IgE, IgG, IgM;

Transport proteins such as retinol binding protein, α-1 microglobulin;

Defensins such as beta-defensin 1, Neutrophil defensins 1, 2 and 3;

Proteins found at the blood brain barrier or in neural tissues, such as melanocortin receptor, myelin, ascorbate transporter;

Transferrin receptor specific ligand-neuropharmaceutical agent fusion proteins (see U.S. Pat. No. 5,977,307); brain capillary endothelial cell receptor, transferrin, transferrin receptor, insulin, insulin-like growth factor 1 (IGF 1) receptor, insulin-like growth factor 2 (IGF 2) receptor, insulin receptor;

Proteins localised to the kidney, such as polycystin, type IV collagen, organic anion transporter K1, Heymann's antigen;

Proteins localised to the liver, for example alcohol dehydrogenase, G250;

Blood coagulation factor X;

α1 antitrypsin;

HNF 1α;

Proteins localised to the lung, such as secretory component (binds IgA);

Proteins localised to the heart, e.g., HSP 27 (this is associated with dilated cardiomyopathy);

Proteins localised to the skin, for example keratin;

Bone specific proteins, such as bone morphogenic proteins (BMPs), which are a subset of the transforming growth factor β superfamily that demonstrate osteogenic activity. Examples include BMP-2, -4, -5, -6, -7 (also referred to as osteogenic protein (OP-1) and -8 (OP-2);

Tumour specific proteins, including human trophoblast antigen, herceptin receptor, oestrogen receptor, cathepsins e.g. cathepsin B (found in liver and spleen);

Disease-specific proteins, such as antigens expressed only on activated T-cells, including (but not limited to):

LAG-3 (lymphocyte activation gene), osteoprotegerin ligand (OPGL) see Nature 402, 304-309; 1999, OX40 (a member of the TNF receptor family, expressed on activated T cells and the only costimulatory T cell molecule known to be specifically up-regulated in human T cell leukaemia virus type-I (HTLV-I)-producing cells.) See J. Immunol. 2000 Jul. 1; 165(1):263-70; Metalloproteases (associated with arthritis/cancers), including CG6512 Drosophila, human paraplegin, human FtsH, human AFG3L2, murine ftsH; angiogenic growth factors, including acidic fibroblast growth factor (FGF-1), basic fibroblast growth factor (FGF-2), Vascular endothelial growth factor/vascular permeability factor (VEGF/VPF), transforming growth factor-a (TGF a), tumor necrosis factor-alpha (TNF-α), angiogenin, interleukin-3 (IL-3), interleukin-8 (IL-8), platelet-derived endothelial growth factor (PD-ECGF), placental growth factor (PIGF), midkine platelet-derived growth factor-BB (PDGF), and fractalkine;

Stress proteins (heat shock proteins)—HSPs are normally found intracellularly. When they are found extracellularly, it is an indicator that a cell has died and spilled out its contents. This unprogrammed cell death (necrosis) only occurs when as a result of trauma, disease or injury and therefore in vivo, extracellular HSPs trigger a response from the immune system that will fight infection and disease. A dual specific ligand which binds to extracellular HSP can be localized to a disease site;

Proteins involved in Fc transport:

Brambell receptor (also known as FcRB). This Fc receptor has two functions, both of which are potentially useful for delivery. The functions are 1) the transport of IgG from mother to child across the placenta, and 2) the protection of IgG from degradation thereby prolonging its serum half life of IgG. It is thought that the receptor recycles IgG from endosome.

### In Vivo Stabilization Using Polymeric Stabilizing Moieties:

In another aspect, a single immunoglobulin variable domain polypeptide containing composition is stabilized in vivo by linkage or association with a (non-polypeptide) polymeric stabilizing moiety. Examples of this type of stabilization are described, for example, in WO99/64460 (Chapman et al.) and EP1,160,255 (King et al.), each of which is incorporated herein by reference. Specifically, these references describe the use of synthetic or naturally-occurring polymer molecules, such as polyalkylene, polyalkenylenes, polyoxyalkylenes or polysaccharides, to increase the in vivo half-life of immunoglobulin polypeptides. A typical example of a stabilizing moiety is polyethylene glycol, or PEG, a polyalkylene. The process of linking PEG to an immunoglobulin polypeptide is described in these references and is referred to herein as “PEGylation.” As described therein, an immunoglobulin polypeptide can be PEGylated randomly, as by attachment of PEG to lysine or other amino acids on the surface of the protein, or site-specifically, e.g., through PEG attachment to an artificially introduced surface cysteine residue. Depending upon the immunoglobulin, it may be preferred to use a non-random method of polymer attachment, because random attachment, by attaching in or near the antigen-binding site or sites on the molecule often alters the affinity or specificity of the molecule for its target antigen.

It is preferred that the addition of PEG or another polymer does not interfere with the antigen-binding affinity or specificity of the antibody variable domain polypeptide. By “does not interfere with the antigen-binding affinity or specificity” is meant that the PEG-linked antibody single variable domain has an IC50 or ND50 which is no more than 10% greater than the IC50 or ND50, respectively, of a non-PEG-linked antibody variable domain having the same antibody single variable domain. In the alternative, the phrase “does not interfere with the antigen-binding affinity or specificity” means that the PEG-linked form of an antibody single variable domain retains at least 90% of the antigen binding activity of the non-PEGylated form of the polypeptide.

The PEG or other polymer useful to increase the in vivo half-life is generally about 5,000 to 50,000 Daltons in size, e.g., about 5,000 kD-10,000 kD, 5,000 kD-15,000 kD, 5,000 kD-20,000 kD, 5,000-25,000 kD, 5,000-30,000 kD, 5,000 kD-35,000 kD, 5,000 kD-40,000 kD, or about 5,000 kD-45,000. The choice of polymer size depends upon the intended use of the complex. For example, where it is desired to penetrate solid tissue, e.g., a tumor, it is advantageous use a smaller polymer, on the order or about 5,000 kD. Where, instead, it is desired to maintain the complex in circulation, larger polymers, e.g., 25,000 kD to 40,000 kD or more can be used.

Homologous Sequences:

The invention encompasses single immunoglobulin variable domain clones and clones with substantial sequence similarity or homology to them that also bind target antigen with high affinity and are soluble at high concentration. As used herein, “substantial” sequence similarity or homology is at least 85% similarity or homology.

Calculations of “homology” or “sequence identity” between two sequences (the terms are used interchangeably herein) are performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “homology” is equivalent to amino acid or nucleic acid “identity”). The percent identity between the two sequences is a function of the number of identical positions shared by the sequences, taking into account the number of gaps, and the length of each gap, which need to be introduced for optimal alignment of the two sequences.

As used herein, sequence “similarity” is a measure of the degree to which amino acid sequences share similar amino acid residues at corresponding positions in an alignment of the sequences. Amino acids are similar to each other where their side chains are similar. Specifically, “similarity” encompasses amino acids that are conservative substitutes for each other. A “conservative” substitution is any substitution that has a positive score in the blosum62 substitution matrix (Hentikoff and Hentikoff, 1992, Proc. Natl. Acad. Sci. USA 89: 10915-10919). By the statement “sequence A is n % similar to sequence B” is meant that n % of the positions of an optimal global alignment between sequences A and B consists of identical amino acids or conservative substitutions. Optimal global alignments can be performed using the following parameters in the Needleman-Wunsch alignment algorithm:

For polypeptides:
Substitution matrix: blosum62.
Gap scoring function: −A−B*LG, where A=11 (the gap penalty), B=1 (the gap length penalty) and LG is the length of the gap.

For nucleotide sequences:
Substitution matrix: 10 for matches, 0 for mismatches.
Gap scoring function: −A−B*LG where A=50 (the gap penalty), B=3 (the gap length penalty) and LG is the length of the gap.

Typical conservative substitutions are among Met, Val, Leu and Ile; among Ser and Thr; among the residues Asp, Glu and Asn; among the residues Gln, Lys and Arg; or aromatic residues Phe and Tyr. In calculating the degree (most often as a percentage) of similarity between two polypeptide sequences, one considers the number of positions at which identity or similarity is observed between corresponding amino acid residues in the two polypeptide sequences in relation to the entire lengths of the two molecules being compared.

Alternatively, the BLAST (Basic Local Alignment Search Tool) algorithm is employed for sequence alignment, with parameters set to default values. The BLAST algorithm “BLAST 2 Sequences” is available at the world wide web site (“www”) of the National Center for Biotechnology Information (“.ncbi”), of the National Library of Medicine (“.nlm”) of the National Institutes of Health (“nih”) of the U.S. government (“.gov”), in the “/blast/” directory, sub-directories “b12seq/b12.html.” This algorithm aligns two sequences for comparison and is described by Tatusova & Madden, 1999, FEMS Microbiol Lett. 174:247-250.

An additional measure of homology or similarity is the ability to hybridize under highly stringent hybridization conditions. Thus, a first sequence encoding a single immunoglobulin variable domain polypeptide is substantially similar to a second coding sequence if the first sequence hybridizes to the second sequence (or its complement) under highly stringent hybridization conditions (such as those described by SAMBROOK et al., Molecular Cloning, Laboratory Manuel, Cold Spring, Harbor Laboratory press, New York). “Highly stringent hybridization conditions” refer to hybridization in 6×SSC at about 45° C., followed by one or more washes in 0.2×SSC, 0.1% SDS at 65° C. “Very highly stringent hybridization conditions” refer to hybridization in 0.5M sodium phosphate, 7% SDS at 65° C., followed by one or more washes at 0.2×SSC, 1% SDS at 65° C.

### Uses of Single Immunoglobulin Variable Domain Polypeptides:

Single immunoglobulin variable domain polypeptides as described herein are useful for a variety of in vivo and in vitro diagnostic, and therapeutic and prophylactic applications. For example, the polypeptides can be incorporated into immunoassays (e.g., ELISAs, RIA, etc.) for the detection of their target antigens in biological samples. Single immunoglobulin variable domain polypeptides can also be of use in, for example, Western blotting applications and in affinity chromatography methods. Such techniques are well known to those of skill in the art.

A very important field of use for single immunoglobulin variable domain polypeptides is the treatment or prophylaxis of diseases or disorders related to the target antigen. Essentially any disease or disorder that is a candidate for treatment or prophylaxis with an antibody preparation is a candidate for treatment or prophylaxis with a single immunoglobulin variable domain polypeptide as described herein. The high binding affinity, human sequence origin, small size and high solubility of the single immunoglobulin variable domain polypeptides described herein render them superior to, for example, full length antibodies or even, for example, scFv for the treatment or prophylaxis of human disease.

Among the diseases or disorders treatable or preventable using the single immunoglobulin variable domain polypeptides described herein are, for example, inflammation, sepsis (including, for example, septic shock, endotoxic shock, Gram negative sepsis and toxic shock syndrome), allergic hypersensitivity, cancer or other hyperproliferative disorders, autoimmune disorders (including, for example, diabetes, rheumatoid arthritis, multiple sclerosis, lupus erythematosis, myasthenia gravis, scleroderma, Crohn's disease, ulcerative colitis, Hashimoto's disease, Graves' disease, Sjögren's syndrome, polyendocrine failure, vitiligo, peripheral neuropathy, graft-versus-host disease, autoimmune polyglandular syndrome type I, acute glomerulonephritis, Addison's disease, adult-onset idiopathic hypoparathyroidism (AOIH), alopecia totalis, amyotrophic lateral sclerosis, ankylosing spondylitis, autoimmune aplastic anemia, autoimmune hemolytic anemia, Behcet's disease, Celiac disease, chronic active hepatitis, CREST syndrome, dermatomyositis, dilated cardiomyopathy, eosinophilia-myalgia syndrome, epidermolisis bullosa acquisita (EBA), giant cell arteritis, Goodpasture's syndrome, Guillain-Barré syndrome, hemochromatosis, Henoch-Schönlein purpura, idiopathic IgA nephropathy, insulin-dependent diabetes mellitus (IDDM), juvenile rheumatoid arthritis, Lambert-Eaton syndrome, linear IgA dermatosis, myocarditis, narcolepsy, necrotizing vasculitis, neonatal lupus syndrome (NLE), nephrotic syndrome, pemphigoid, pemphigus, polymyositis, primary sclerosing cholangitis, psoriasis, rapidly-progressive glomerulonephritis (RPGN), Reiter's syndrome, stiff-man syndrome and thyroiditis), effects of infectious disease (e.g., by limiting inflammation, cachexia or cytokine-mediated tissue damage), transplant rejection and graft versus host disease, pulmonary disorders (e.g., respiratory distress syndrome, shock lung, chronic pulmonary inflammatory disease, pulmonary sarcoidosis, pulmonary fibrosis and silicosis), cardiac disorders (e.g., ischemia of the heart, heart insufficiency), inflammatory bone disorders and bone resorption disease, hepatitis (including alcoholic hepatitis and viral hepatitis), coagulation disturbances, reperfusion injury, keloid formation, scar tissue formation and pyrexia.

Cancers can be treated, for example, by targeting one or more molecules, e.g., cytokines or growth factors, cell surface receptors or antigens, or enzymes, necessary for the growth and/or metabolic activity of the tumor, or, for example, by using a single immunoglobulin variable domain polypeptide specific for a tumor-specific or tumor-enriched antigen to target a liked cytotoxic or apoptosis-inducing agent to the tumor cells. Other diseases or disorders, e.g., inflammatory or autoimmune disorders, can be treated in a similar manner, by targeting one or more mediators of the pathology with a neutralizing single immunoglobulin variable domain polypeptide as described herein. Most commonly, such mediators will be, for example, endogenous cytokines (e.g., TNF-α) or their receptors that mediate inflammation or other tissue damage.

Pharmaceutical Compositions, Dosage and Administration

The single immunoglobulin variable domain polypeptides of the invention can be incorporated into pharmaceutical compositions suitable for administration to a subject. Typically, the pharmaceutical composition comprises a single immunoglobulin variable domain polypeptide and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. The term “pharmaceutically acceptable carrier” excludes tissue culture medium comprising bovine or horse serum. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable substances include minor amounts of auxiliary substances such as wetting or emulsifying agents, preservatives or buffers, which enhance the shelf life or effectiveness of the single immunoglobulin variable domain polypeptide.

The compositions as described herein may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application. Typical preferred compositions are in the form of injectable or infusible solutions, such as compositions similar to those used for passive immunization of humans with other antibodies. The preferred mode of administration is parenteral (e.g., intravenous, subcutaneous, intraperitoneal, intramuscular).

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the active compound in the required amount in an appropriate solvent with one or a combination of ingredients enumerated above, as required, followed by filter sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants.

The single immunoglobulin variable domain polypeptides described herein can be administered by a variety of methods known in the art, although for many therapeutic applications, the preferred route/mode of administration is intravenous injection or infusion. The polypeptide can also be administered by intramuscular or subcutaneous injection. Preparations according to the invention include concentrated solutions of the single immunoglobulin variable domain, e.g., solutions of at least 5 mg/ml (˜417 μM) in aqueous solution (e.g., PBS), and preferably at least 10 mg/ml (˜833 μM), 20 mg/ml (˜1.7 mM), 25 mg/ml (˜2.1 mM), 30 mg/ml (˜2.5 mM), 35 mg/ml (˜2.9 mM), 40 mg/ml (˜3.3 mM), 45 mg/ml (˜3.75 mM), 50 mg/ml (˜4.2 mM), 55 mg/ml (˜4.6 mM) 60 mg/ml (˜5.0 mM), 65 mg/ml (˜5.4 mM), 70 mg/ml (˜5.8 mM), 75 mg/ml (˜6.3 mM), 100 mg/ml (˜8.33 mM), 150 mg/ml (˜12.5 mM), 200 mg/ml (˜16.7 mM) or higher. In some embodiments, preparations can be, for example, 250 mg/ml (˜20.8 mM), 300 mg/ml (˜25 mM), 350 mg/ml (29.2 mM) or even higher, but be diluted down to 200 mg/ml or below prior to use.

As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Single immunoglobulin variable domains are well suited for formulation as extended release preparations due, in part, to their small size—the number of moles per dose can be significantly higher than the dosage of, for example, full sized antibodies. Biodegradable, biocompatible polymers can be used, such as ethylene vinyl acetate, polyanhydrides, polyglycolic acid, collagen, polyorthoesters, and polylactic acid. Prolonged absorption of injectable compositions can be brought about by including in the composition an agent that delays absorption, for example, monostearate salts and gelatin. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978. Additional methods applicable to the controlled or extended release of polypeptide agents such as the single immunoglobulin variable domain polypeptides disclosed herein are described, for example, in U.S. Pat. Nos. 6,306,406 and 6,346,274, as well as, for example, in U.S. Patent Application Nos. US20020182254 and US20020051808, all of which are incorporated herein by reference.

In certain embodiments, a single immunoglobulin variable domain polypeptide may be orally administered, for example, with an inert diluent or an assimilable edible carrier. The compound (and other ingredients, if desired) may also be enclosed in a hard or soft shell gelatin capsule, compressed into tablets, or incorporated directly into the individual's diet. For oral therapeutic administration, the compounds may be incorporated with excipients and used in the form of ingestible tablets, buccal tablets, troches, capsules, elixirs, suspensions, syrups, wafers, and the like. To administer a compound of the invention by other than parenteral administration, it may be necessary to coat the compound with, or co-administer the compound with, a material to prevent its inactivation.

Additional active compounds can also be incorporated into the compositions. In certain embodiments, a single immunoglobulin variable domain polypeptide is coformulated with and/or coadministered with one or more additional therapeutic agents. For example, a single immunoglobulin variable domain polypeptide may be coformulated and/or coadministered with one or more additional antibodies that bind other targets (e.g., antibodies that bind other cytokines or that bind cell surface molecules), or, for example, one or more cytokines. Such combination therapies may utilize lower dosages of the administered therapeutic agents, thus avoiding possible toxicities or complications associated with the various monotherapies.

The pharmaceutical compositions of the invention may include a “therapeutically effective amount” or a “prophylactically effective amount” of a single immunoglobulin variable domain polypeptide. A “therapeutically effective amount” refers to an amount effective, at dosages and for periods of time necessary, to achieve the desired therapeutic result. A therapeutically effective amount of the single immunoglobulin variable domain polypeptide may vary according to factors such as the disease state, age, sex, and weight of the individual, and the ability of the single immunoglobulin variable domain polypeptide to elicit a desired response in the individual. A therapeutically effective amount is also one in which any toxic or detrimental effects of the antibody or antibody portion are outweighed by the therapeutically beneficial effects. A “prophylactically effective amount” refers to an amount effective, at dosages and for periods of time necessary, to achieve the desired prophylactic result. Typically, because a prophylactic dose is used in subjects prior to or at an earlier stage of disease, the prophylactically effective amount will be less than the therapeutically effective amount.

Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation. It is advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier.

A non-limiting range for a therapeutically or prophylactically effective amount of a single immunoglobulin variable domain polypeptide is 0.1-20 mg/kg, more preferably 1-10 mg/kg. It is to be noted that dosage values may vary with the type and severity of the condition to be alleviated. It is to be further understood that for any particular subject, specific dosage regimens should be adjusted over time according to the individual need and the professional judgment of the administering clinician.

The efficacy of treatment with a single immunoglobulin variable domain polypeptide as described herein is judged by the skilled clinician on the basis of improvement in one or more symptoms or indicators of the disease state or disorder being treated. An improvement of at least 10% (increase or decrease, depending upon the indicator being measured) in one or more clinical indicators is considered “effective treatment,” although greater improvements are preferred, such as 20%, 30%, 40%, 50%, 75%, 90%, or even 100%, or, depending upon the indicator being measured, more than 100% (e.g., two-fold, three-fold, ten-fold, etc., up to and including attainment of a disease-free state. Indicators can be physical measurements, e.g., enzyme, cytokine, growth factor or metabolite levels, rate of cell growth or cell death, or the presence or amount of abnormal cells. One can also measure, for example, differences in the amount of time between flare-ups of symptoms of the disease or disorder (e.g., for remitting/relapsing diseases, such as multiple sclerosis). Alternatively, non-physical measurements, such as a reported reduction in pain or discomfort or other indicator of disease status can be relied upon to gauge the effectiveness of treatment. Where non-physical measurements are made, various clinically acceptable scales or indices can be used, for example, the Crohn's Disease Activity Index, or CDAI (Best et al., 1976, Gastroenterology 70: 439), which combines both physical indicators, such as hematocrit and the number of liquid or very soft stools, among others, with patient-reported factors such as the severity of abdominal pain or cramping and general well-being, to assign a disease score.

As the term is used herein, “prophylaxis” performed using a composition as described herein is “effective” if the onset or severity of one or more symptoms is delayed or reduced by at least 10%, or abolished, relative to such symptoms in a similar individual (human or animal model) not treated with the composition.

Accepted animal models of human disease can be used to assess the efficacy of a single immunoglobulin variable domain polypeptide as described herein for treatment or prophylaxis of a disease or disorder. Examples of such disease models include, for example: a guinea pig model for allergic asthma as described by Savoie et al., 1995, Am. J. Respir. Cell Biol. 13: 133-143; an animal model for multiple sclerosis, experimental autoimmune encephalomyelitis (EAE), which can be induced in a number of species, e.g., guinea pig (Suckling et al., 1984, Lab. Anim. 18: 36-39), Lewis rat (Feurer et al., 1985, J. Neuroimmunol. 10: 159-166), rabbits (Brenner et al., 1985, Isr. J. Med. Sci. 21: 945-949), and mice (Zamvil et al., 1985, Nature 317: 355-358); animal models known in the art for diabetes, including models for both insulin-dependent diabetes mellitus (IDDM) and non-insulin-dependent diabetes mellitus (NIDDM)—examples include the non-obese diabetic (NOD) mouse (e.g., Li et al., 1994, Proc. Natl. Acad. Sci. U.S.A. 91: 11128-11132), the BB/Drat (Okwueze et al., 1994, Am. J. Physiol. 266: R572-R577), the Wistar fatty rat (Jiao et al., 1991, Int. J. Obesity 15: 487-495), and the Zucker diabetic fatty rat (Lee et al., 1994, Proc. Natl. Acad. Sci. U.S.A. 91” 10878-10882); animal models for prostate disease (Loweth et al., 1990, Vet. Pathol. 27: 347-353), models for atherosclerosis (numerous models, including those described by Chao et al., 1994, J. Lipid Res. 35: 71-83; Yoshida et al., 1990, Lab. Anim. Sci. 40: 486-489; and Hara et al., 1990, Jpn. J. Exp. Med. 60: 315-318); nephrotic syndrome (Ogura et al., 1989, Lab. Anim. 23: 169-174); autoimmune thyroiditis (Dietrich et al., 1989, Lab. Anim. 23: 345-352); hyperuricemia/gout (Wu et al., 1994, Proc. Natl. Acad. Sci. U.S.A. 91: 742-746), gastritis (Engstrand et al., 1990, Infect. Immunity 58: 1763-1768); proteinuria/kidney glomerular defect (Hyun et al., 1991, Lab. Anim. Sci. 41:442-446); food allergy (e.g., Ermel et al., 1997, Lab. Anim. Sci. 47: 40-49; Knippels et al., 1998, Clin. Exp. Allergy 28: 368-375; Adel-Patient et al., 2000, J. Immunol. Meth. 235: 21-32; Kitagawa et al., 1995, Am. J. Med. Sci. 310: 183-187; Panush et al., 1990, J. Rheumatol. 17: 285-290); rheumatoid disease (Mauri et al., 1997, J. Immunol. 159: 5032-5041; Saegusa et al., 1997, J. Vet. Med. Sci. 59: 897-903; Takeshita et al., 1997, Exp. Anim. 46: 165-169); osteoarthritis (Rothschild et al., 1997, Clin. Exp. Rheumatol. 15: 45-51; Matyas et al., 1995, Arthritis Rheum. 38: 420-425); lupus (Walker et al., 1983, Vet. Immunol. Immunopathol. 15: 97-104; Walker et al., 1978, J. Lab. Clin. Med. 92: 932-943); and Crohn's disease (Dieleman et al., 1997, Scand. J. Gastroenterol. Supp. 223: 99-104; Anthony et al., 1995, Int. J. Exp. Pathol. 76: 215-224; Osborne et al., 1993, Br. J. Surg. 80: 226-229). Other animal models are known to those skilled in the art.

Whereas the single immunoglobulin variable domain polypeptides described herein must bind a human antigen with high affinity, where one is to evaluate its effect in an animal model system, the polypeptide must cross-react with one or more antigens in the animal model system, preferably at high affinity. One of skill in the art can readily determine if this condition is satisfied for a given animal model system and a given single immunoglobulin variable domain polypeptide. If this condition is satisfied, the efficacy of the single immunoglobulin variable domain polypeptide can be examined by administering it to an animal model under conditions which mimic a disease state and monitoring one or more indicators of that disease state for at least a 10% improvement.

### EXAMPLES

### Example 1

### Selection of a Collection of Single Domain Antibodies (dAbs) Directed Against Human Serum Albumin (HSA) and Mouse Serum Albumin (MSA)

The generation of a library of V_{H} or V_{L} sequences with diversity at specified residues is described in WO 99/20749, which is incorporated herein by reference. For the identification of single domain antibodies specific for HSA and MSA, the same approach was used to generate the following three different libraries, each based on a single human framework for V_{H} or V_{κ}, with side chain diversity encoded by NNK codons incorporated into CDRs 1, 2 and 3:

V_{H} (see FIGS. 1 and 2: sequence of dummy V_{H} based on V3-23/DP47 and JH4b) or Vκ (see FIG. 3: sequence of dummy Vκ based on o12/o2/DPK9 and Jk1) with side chain diversity encoded by NNK codons incorporated in complementarity determining regions (CDR1, CDR2 and CDR3).
Library 1 (V_{H}, based on V3-23/DP47 and JH4b; see FIG. 1):

| | |
|---|---|
| Diversity at positions: | H30, H31, H33, H35, H50, H52, H52a, |
| | H53, H55, H56, H58, H95, H97, H98. |
| Library size: | 6.2 × 10⁹ |

Library 2 (V_{H}, based on V3-23/DP47 and JH4b; see FIG. 2):

| | |
|---|---|
| Diversity at positions: | H30, H31, H33, H35, H50, H52, H52a, |
| | H53, H55, H56, H58, H95, |
| | H97, H98, H99, H100, H100a, H100b. |
| Library size: | 4.3 × 10⁹ |

Library 3 (Vκ, based on O12/O2/DPK9 and J_{κ}1; see FIG. 3):

| | |
|---|---|
| Diversity at positions: | L30, L31, L32, L34, L50, L53, |
| | L91, L92, L93, L94, L96 |
| Library size: | 2 × 10⁹ |

The V_{H} and Vκ libraries have been preselected for binding to generic ligands protein A and protein L respectively so that the majority of clones in the unselected libraries are functional. The sizes of the libraries shown above correspond to the sizes after preselection.

Two rounds of selection were performed on serum albumin using each of the libraries separately. For each selection, antigen was coated on immunotube (nunc) in 4 ml of PBS at a concentration of 100 μg/ml. In the first round of selection, each of the three libraries was panned separately against HSA (Sigma) and MSA (Sigma). In the second round of selection, phage from each of the six first round selections was panned against (i) the same antigen again (e.g. 1^{st} round MSA, 2^{nd} round MSA) and (ii) against the reciprocal antigen (e.g. 1^{st} round MSA, 2^{nd} round HSA) resulting in a total of twelve 2^{nd} round selections. In each case, after the second round of selection 48 clones were tested for binding to HSA and MSA. Soluble dAb fragments were produced as described for scFv fragments by Harrison et al, Methods Enzymol. 1996; 267:83-109 and standard ELISA protocol was followed (Hoogenboom et al., 1991, Nucleic Acids Res. 19: 4133) except that 2% tween PBS was used as a blocking buffer and bound dAbs were detected with either protein L-HRP (Sigma) (for the Vκs) and protein A-HRP (Amersham Pharmacia Biotech) (for the V_{H}s).

dAbs that gave a signal above background indicating binding to MSA, HSA or both were tested in ELISA insoluble form for binding to plastic alone but all were specific for serum albumin. Clones were then sequenced (see table below) revealing that 21 unique dAb sequences had been identified. The minimum similarity between the Vκ dAb clones selected was 86.25% ((69/80)×100—the result when all the diversified residues are different, e.g. clones 24 and 34). The minimum similarity between the V_{H} dAb clones selected was 94% ((127/136)×100).

Next, the serum albumin binding dAbs were tested for their ability to capture biotinylated antigen from solution. The ELISA protocol (as above) was followed except that the ELISA plate was coated with 1 μg/ml protein L (for the Vκ clones) and 1 μg/ml protein A (for the V_{H} clones). Soluble dAb was captured from solution as in the protocol and detection was with biotinylated MSA or HSA and streptavidin HRP. The biotinylated MSA and HSA had been prepared according to the manufacturer's instructions, with the aim of achieving an average of 2 biotins per serum albumin molecule. Twenty four clones were identified that captured biotinylated MSA from solution in the ELISA (Table 1). Two of these (clones 2 and 38 below) also captured biotinylated HSA. Next, the dAbs were tested for their ability to bind MSA coated on a CM5™ Biacore surface plasmon resonance (SPR) chip. Eight clones were found that bound MSA on the Biacore chip.

TABLE 1

| dAb (all | | | | | | |
|---|---|---|---|---|---|---|
| capture | | | | | Binds | Captures |
| biotinylated | | | | | MSA in | biotinylated |
| MSA) | H or κ | CDR1 | CDR2 | CDR3 | biacore? | HSA? |
| Vκ library 3 | | XXXLX | XASXLQS | QQXXXXPXT | | |
| template | κ | SEQ ID NO: 92 | SEQ ID NO: 93 | SEQ ID NO: 94 | | |
| (dummy) | | SSYLN | RASPLQS | QQTYSVPPT | | |
| 2, 4, 7, 41, | κ | SEQ ID NO 95 | SEQ ID NO: 96 | SEQ ID NO: 97 | | ✓all 4 bind |
| | | SSYLN | RASPLQS | QQTYRIPPT | | |
| 38, 54 | κ | SEQ ID NO: 98 | SEQ ID NO: 99 | SEQ ID NO: 100 | | ✓both bind |
| | | FKSLK | NASYLQS | QQVVYWPVT | | |
| 46, 47, 52, 56 | κ | SEQ ID NO: 101 | SEQ ID NO: 102 | SEQ ID NO: 103 | | |
| | | YYHLK | KASTLQS | QQVRKVPRT | | |
| 13, 15 | κ | SEQ ID NO: 104 | SEQ ID NO: 105 | SEQ ID NO: 106 | | |
| | | RRYLK | QASVLQS | QQGLYPPIT | | |
| 30, 35 | κ | SEQ ID NO: 107 | SEQ ID NO: 108 | SEQ ID NO: 109 | | |
| | | YNWLK | RASSLQS | QQNVVIPRT | | |
| 19, | κ | SEQ ID NO: 110 | SEQ ID NO: 111 | SEQ ID NO: 112 | | |
| | | LWHLR | HASLLQS | QQSAVYPKT | | |
| 22, | κ | SEQ ID NO: 113 | SEQ ID NO: 114 | SEQ ID NO: 115 | | |
| | | FRYLA | HASHLQS | QQRLLYPKT | | |
| 23, | κ | SEQ ID NO: 116 | SEQ ID NO: 117 | SEQ ID NO: 118 | | |
| | | FYHLA | PASKLQS | QQRARWPRT | | |
| 24, | κ | SEQ ID NO: 119 | SEQ ID NO: 120 | SEQ ID NO: 121 | | |
| | | IWHLN | RASRLQS | QQVARVPRT | | |
| 31, | κ | SEQ ID NO: 122 | SEQ ID NO: 123 | SEQ ID NO: 124 | | |
| | | YRYLR | KASSLQS | QQYVGYPRT | | |
| 33, | κ | SEQ ID NO: 125 | SEQ ID NO: 126 | SEQ ID NO: 127 | | |
| | | LKYLK | NASHLQS | QQTTYYPIT | | |
| 34, | κ | SEQ ID NO: 128 | SEQ ID NO: 129 | SEQ ID NO: 130 | | |
| | | LRYLR | KASWLQS | QQVLYYPQT | | |
| 53, | κ | SEQ ID NO: 131 | SEQ ID NO: 132 | SEQ ID NO: 133 | | |
| | | LRSLK | AASRLQS | QQVVYWPAT | | |
| 11, | κ | SEQ ID NO: 134 | SEQ ID NO: 135 | SEQ ID NO: 136 | ✓ | |
| | | FRHLK | AASRLQS | QQVALYPKT | | |
| 12, | κ | SEQ ID NO: 137 | SEQ ID NO: 138 | SEQ ID NO: 139 | ✓ | |
| | | RKYLR | TASSLQS | QQNLFWPRT | | |
| 17, | κ | SEQ ID NO: 140 | SEQ ID NO: 141 | SEQ ID NO: 142 | ✓ | |
| | | RRYLN | AASSLQS | QQMLFYPKT | | |
| 18, | κ | SEQ ID NO: 143 | SEQ ID NO: 144 | SEQ ID NO: 145 | ✓ | |
| | | IKHLK | GASRLQS | QQGARWPQT | | |
| 16,21 | κ | SEQ ID NO: 146 | SEQ ID NO: 147 | SEQ ID NO: 148 | ✓ | |
| | | YYHLK | KASTLQS | QQVRKVPRT | | |
| 25,26 | κ | SEQ ID NO: 149 | SEQ ID NO: 150 | SEQ ID NO: 151 | ✓ | |
| | | YKHLK | NASHLQS | QQVGRYPKT | | |
| 27, | κ | SEQ ID NO: 152 | SEQ ID NO: 153 | SEQ ID NO: 154 | ✓ | |
| | | FKSLK | NASYLQS | QQVVYWPVT | | |
| 55, | κ | SEQ ID NO: 155 | SEQ ID NO: 156 | SEQ ID NO: 157 | ✓ | |
| VH library 1 | | XXYXXX | XIXXXGXXTXYADSVKG | XXXX(XXXX)FDY | | |
| (and 2) | H | SEQ ID NO: 158 | SEQ ID NO: 159 | SEQ ID NO: 160 | | |
| template | | WVYQMD | SISAFGAKTLYADSVKG | LSGKFDY | | |
| (dummy) | | | | | | |
| 8, 10 | H | SEQ ID NO: 161 | SEQ ID NO: 162 | SEQ ID NO: 163 | | |
| | | WSYQMT | SISSFSSSTLYADSVKS | GRDHNYSLFDY | | |
| 36, | H | SEQ ID NO: 164 | SEQ ID NO: 165 | SEQ ID NO: 166 | | |

In all cases the frameworks were identical to the frameworks in the corresponding dummy sequence, with diversity in the CDRs as indicated in the table above.

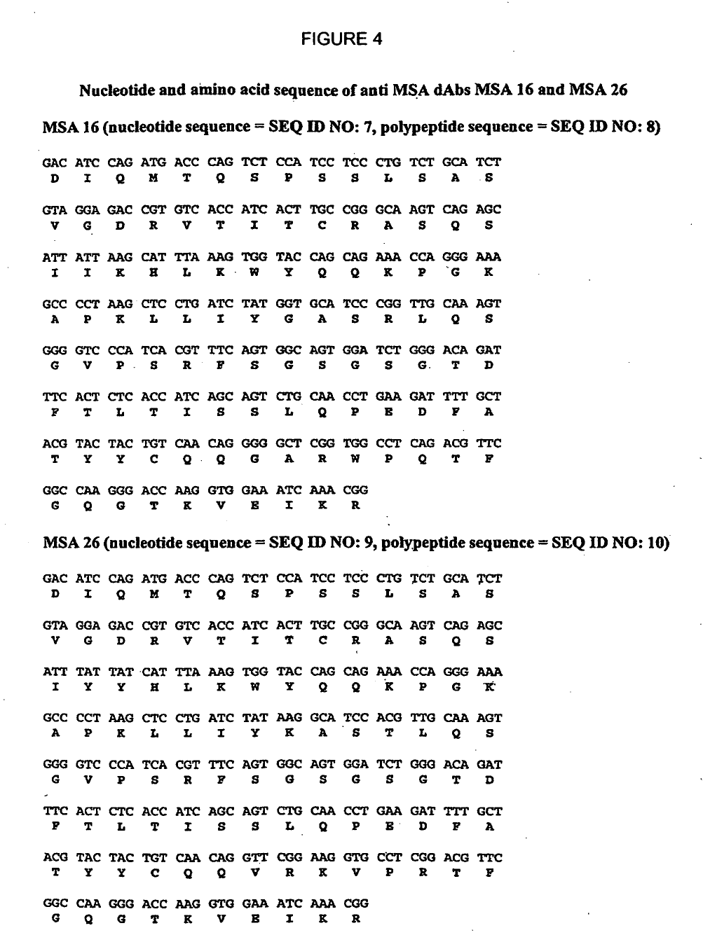

Of the eight clones that bound MSA on the Biacore chip, two clones that are highly expressed in E. coli (clones MSA16 and MSA26) were chosen for further study (see Example 2). Full nucleotide and amino acid sequences for MSA16 and 26 are given in FIG. 4.

### Example 2

### Determination of Affinity and Serum Half-Life in Mouse of MSA-Binding dAbs MSA16 and MSA26

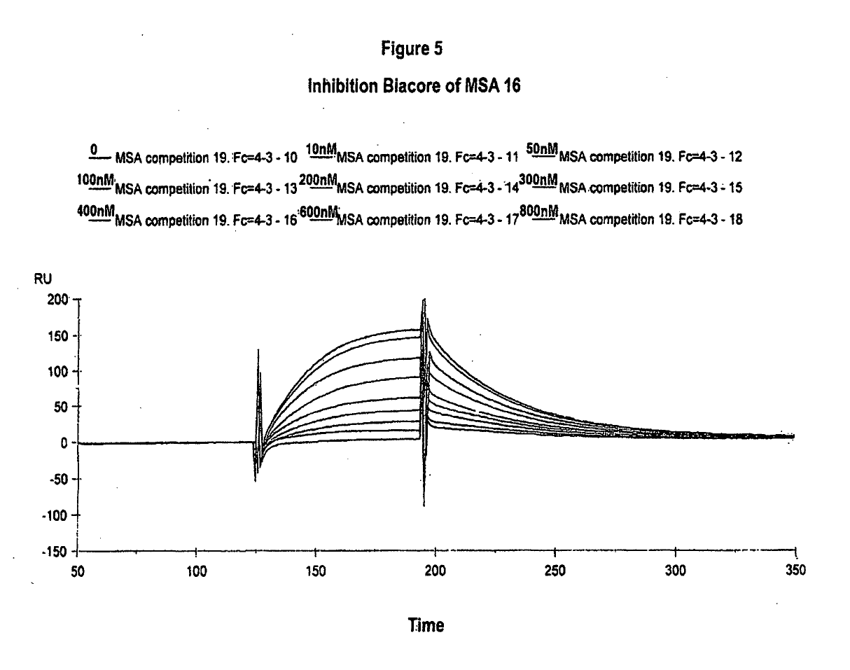

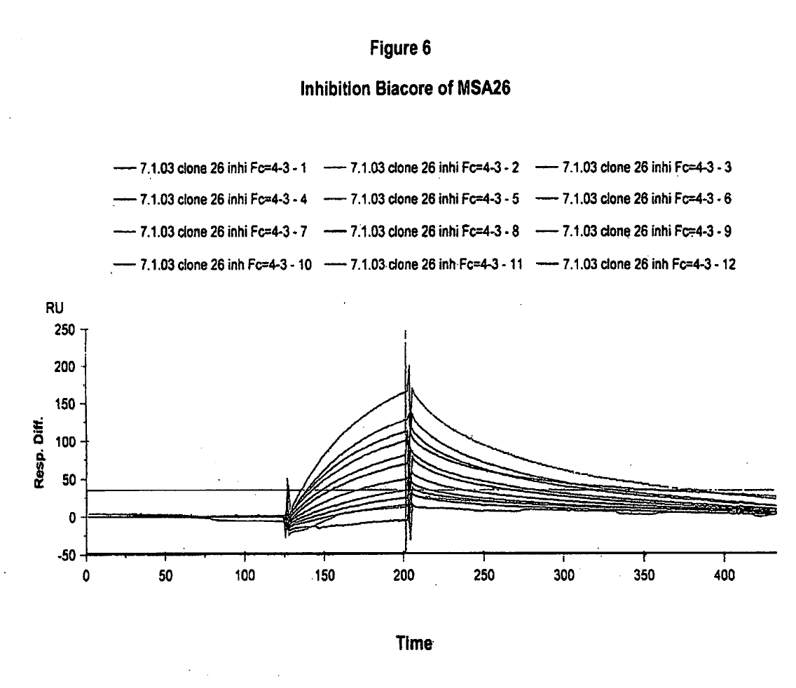

dAbs MSA16 and MSA26 were expressed in the periplasm of E. coli and purified using batch absorption to protein L-agarose affinity resin (Affitech, Norway) followed by elution with glycine at pH 2.2. The purified dAbs were then analysed by inhibition surface plasmon resonance to determine K_{d}. Briefly, purified MSA16 and MSA26 were tested to determine the concentration of dAb required to achieve 200RUs of response on a Biacore CM5™ SPR chip coated with a high density of MSA. Once the required concentrations of dAb had been determined, MSA antigen at a range of concentrations around the expected K_{d} was premixed with the dAb and incubated overnight. Binding of dAb to the MSA coated SPR chip in each of the premixes was then measured at a high flow-rate of 30 μl/minute. The resulting curves were used to create Klotz plots, which gave an estimated K_{d} of 200 nM for MSA16 (FIG. 5) and 70 nM for MSA 26 (FIG. 6).

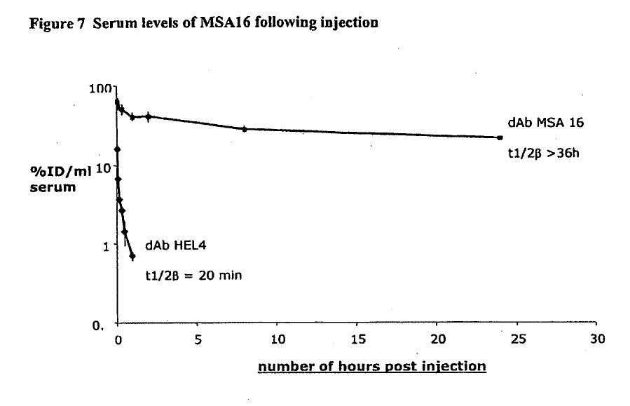

Next, clones MSA16 and MSA26 were cloned into an expression vector with the HA tag (nucleic acid sequence: TATCCTTATGATGTTCCTGATTATGCA (SEQ ID NO: 167) and amino acid sequence: YPYDVPDYA (SEQ ID NO: 168)) and 2-10 mg quantities were expressed in E. coli and purified from the supernatant with protein L-agarose affinity resin (Affitech, Norway) and eluted with glycine at pH 2.2. Serum half life of the dAbs was determined in mouse. MSA26 and MSA16 were dosed as single i.v. injections at approx 1.5 mg/kg into CD1 mice. Analysis of serum levels was by goat anti-HA (Abcam, UK) capture and protein L-HRP (invitrogen) detection ELISA which was blocked with 4% Marvel. Washing was with 0.05% Tween PBS. Standard curves of known concentrations of dAb were set up in the presence of 1× mouse serum to ensure comparability with the test samples. Modeling with a 2 compartment model showed MSA-26 had a t1/2α of 0.16 hr, a t1/2β of 14.5 hr and an area under the curve (AUC) of 465 hr.mg/ml (data not shown) and MSA-16 had a t1/2α of 0.98 hr, a t1/2β of 36.5 hr and an AUC of 913 hr.mg/ml (FIG. 7). Both anti-MSA clones had considerably lengthened half life compared with HEL4 (an anti-hen egg white lysozyme dAb) which had a t1/2α of 0.06 hr, and a t1/2β of 0.34 hr.

### Example 3

### Identification of Single Immunoglobulin Variable Domain Polypeptides Specific for Hen Egg Lysozyme, TNF-α and TNF Receptor

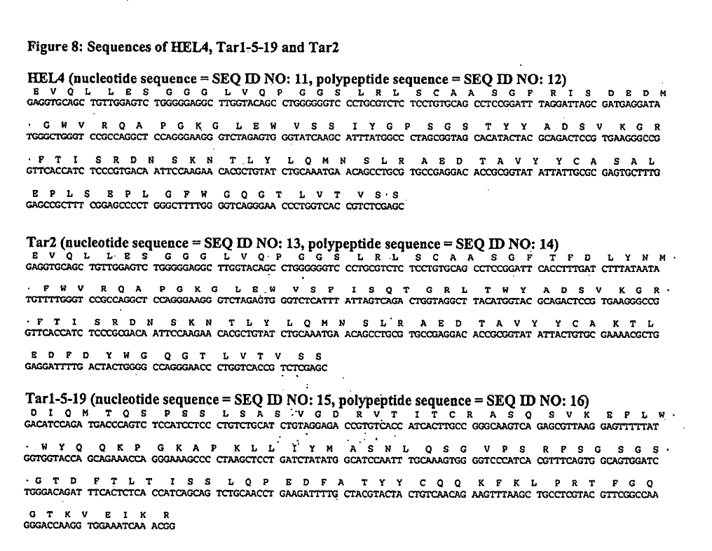

A number of single immunoglobulin variable domain polypeptides that bind hen egg lysozyme (HEL), TNF-α and TNF Receptor (p55) were identified from dAb libraries similar to those described in Example 1. The HEL-specific and TNF Receptor dAbs were identified from a DP47-based V_{H} library, and the TNF-α dAbs were identified from a V_{κ} library based on DPK9. Representative nucleic acid and amino acid sequences are provided in FIG. 8.

### Example 4

### Dimerization of TNF-α Specific Single Immunoglobulin Variable Domain Polypeptide

Homodimers of the single immunoglobulin variable domain polypeptides described herein can increase the antigen binding strength of the polypeptides, most likely through the avidity effect. This was investigated by homodimerization of the TAR1-5-19 dAb isolated as described above and provided in FIG. 8.

The TAR1-5-19 dAb was engineered to have a free cysteine at its C terminus. Expression of the cysteine-modified dAb in E. coli resulted in a mixture of monomeric and dimeric (disulfide-bonded) forms.

The following oligonucleotides were used to specifically PCR TAR1-5-19 with a SalI and BamHI sites for cloning and also to introduce a C-terminal cysteine residue

| Forward primer | |
|---|---|
| 5′-TGGAGCGCGTCGACGGACATCCAGATGACCCAGTCTCCA-3′ | (SEQ ID NO: 169) |
| Reverse primer | |
| 5′-TTAGCAGCCGGATCCTTATTAGCACCGTTTGATTTCCAC-3′ | (SEQ ID NO: 170) |
| 10 SalI | |
| 10 -------- | |

| | |
|---|---|
| | Trp Ser Ala Ser Thr Asp* Ile Gln Met Thr Gln Ser Pro Ser Ser Leu Ser Ala Ser Val |
| 1 | TGG AGC GCG TCG ACG GAC ATC CAG ATG ACC CAG TCT CCA TCC TCT CTG TCT GCA TCT GTA |
| | ACC TCG CGC AGC TGC CTG TAG GTC TAC TGG GTC AGA GGT AGG AGA GAC AGA CGT AGA CAT |
| | Gly Asp Arg Val Thr Ile Thr Cys Arg Ala Ser Gln Ser Ile Asp Ser Tyr Leu His Trp |
| 61 | GGA GAC CGT GTC ACC ATC ACT TGC CGG GCA AGT CAG AGC ATT GAT AGT TAT TTA CAT TGG |
| | CCT CTG GCA CAG TGG TAG TGA ACG GCC CGT TCA GTC TCG TAA CTA TCA ATA AAT GTA ACC |
| | Tyr Gln Gln Lys Pro Gly Lys Ala Pro Lys Leu Leu Ile Tyr Ser Ala Ser Glu Leu Gln |
| 121 | TAC CAG CAG AAA CCA GGG AAA GCC CCT AAG CTC CTG ATC TAT AGT GCA TCC GAG TTG CAA |
| | ATG GTC GTC TTT GGT CCC TTT CGG GGA TTC GAG GAC TAG ATA TCA CGT AGG CTC AAC GTT |
| | Ser Gly Val Pro Ser Arg Phe Ser Gly Ser Gly Ser Gly Thr Asp Phe Thr Leu Thr Ile |
| 181 | AGT GGG GTC CCA TCA CGT TTC AGT GGC AGT GGA TCT GGG ACA GAT TTC ACT CTC ACC ATC |
| | TCA CCC CAG GGT AGT GCA AAG TCA CCG TCA CCT AGA CCC TGT CTA AAG TGA GAG TGG TAG |
| | Ser Ser Leu Gln Pro Glu Asp Phe Ala Thr Tyr Tyr Cys Gln Gln Val Val Trp Arg Pro |
| 241 | AGC AGT CTG CAA CCT GAA GAT TTT GCT ACG TAC TAC TGT CAA CAG GTT GTG TGG CGT CCT |
| | TCG TCA GAC GTT GGA CTT CTA AAA CGA TGC ATG ATG ACA GTT GTC CAA CAC ACC GCA GGA |
| BamHI | |
| -------- | |
| | Phe Thr Phe Gly Gln Gly Thr Lys Val Glu Ile Lys Arg Cys *** *** Gly Ser Gly |
| 301 | TTT ACG TTC GGC CAA GGG ACC AAG GTG GAA ATC AAA CGG TGC TAA TAA GGA TCC GGC |
| | AAA TGC AAG CCG GTT CCC TGG TTC CAC CTT TAG TTT GCC ACG ATT ATT CCT AGG CCG |

(SEQ ID Nos: 171 (nucleotide), 172 (amino acid);
*start of TAR1-5-19CYS sequence)

The PCR reaction (50 μl volume) was set up as follows: 200 μM dNTP's, 0.4 μM of each primer, 5 μl of 10× PfuTurbo buffer (Stratagene), 100 ng of template plasmid (encoding TAR1-5-19), 1 μl of PfuTurbo enzyme (Stratagene) and the volume adjusted to 50 μl using sterile water. The following PCR conditions were used: initial denaturing step 94° C. for 2 mins, then 25 cycles of 94° C. for 30 secs, 64° C. for 30 sec and 72° C. for 30 sec. A final extension step was also included of 72° C. for 5 mins. The PCR product was purified and digested with SalI and BamHI and ligated into the vector which had also been cut with the same restriction enzymes. Correct clones were verified by DNA sequencing.

Expression and Purification of TAR1-5-19CYS

TAR1-5-19CYS vector was transformed into BL21 (DE3) pLysS chemically competent cells (Novagen) following the manufacturer's protocol. Cells carrying the dAb plasmid were selected using 100 μg/mL carbenicillin and 37 μg/mL chloramphenicol. Cultures were set up in 2 L baffled flasks containing 500 mL of terrific broth (Sigma-Aldrich), 100 μg/mL carbenicillin and 37 μg/mL chloramphenicol. The cultures were grown at 30° C. at 200 rpm to an O.D.₆₀₀ of 1-1.5 and then induced with 1 mM IPTG (isopropyl-β-D-thiogalactopyranoside, from Melford Laboratories). The expression of the dAb was allowed to continue for 12-16 hrs at 30° C. It was found that most of the dAb was present in the culture media. Therefore, the cells were separated from the media by centrifugation (8,000×g for 30 mins), and the supernatant was used to purify the dAb. Per litre of supernatant, 30 mL of Protein L agarose (Affitech) was added and the dAb allowed to batch bind with stirring for 2 hours. The resin was then allowed to settle under gravity for a further hour before the supernatant was siphoned off. The agarose was then packed into a XK 50 column (Amersham Phamacia) and was washed with 10 column volumes of PBS. The bound dAb was eluted with 100 mM glycine pH 2.0 and protein containing fractions were then neutralized by the addition of ⅕ volume of 1 M Tris pH 8.0. Per litre of culture supernatant, 20 mg of pure protein was isolated, which contained a 50:50 ratio of monomer to dimer.

Separation of TAR1-5-19CYS Monomer from the TAR1-5-19CYS Dimer

Cation exchange chromatography was used to separate monomers from homodimers. Prior to cation exchange separation, the mixed monomer/dimer sample was buffer exchanged into 50 mM sodium acetate buffer pH 4.0 using a PD-10 column (Amersham Pharmacia), following the manufacturer's guidelines. The sample was then applied to a 1 mL Resource S cation exchange column (Amersham Pharmacia), which had been pre-equilibrated with 50 mM sodium acetate pH 4.0. The monomer and dimer were separated using the following salt gradient in 50 mM sodium acetate pH 4.0:
150 to 200 mM sodium chloride over 15 column volumes
200 to 450 mM sodium chloride over 10 column volumes
450 to 1000 mM sodium chloride over 15 column volumes
   Fractions containing dimer only were identified using SDS-PAGE and then pooled and the pH increased to 8 by the addition of ⅕ volume of 1M Tris pH 8.0.

In Vitro Functional Binding Assay: TNF Receptor Assay and Cell Assay

The affinity of the dimer for human TNFα was determined using the TNF receptor and cell assay. IC₅₀ in the receptor assay was approximately 0.3-0.8 nM; ND₅₀ in the cell assay was approximately 3-8 nM.

Other possible TAR1-5-19CYS dimer formats include, for example, PEG dimers and custom synthetic maleimide dimers. Nektar (Shearwater) offer a range of bi-maleimide PEGs [mPEG2-(MAL)2 or mPEG-(MAL)2] which would allow the monomer to be formatted as a dimer, with a small linker separating the dAbs and both being linked to a PEG ranging in size from 5 to 40 kDa. It has been shown that the 5 kDa mPEG-(MAL)2 (i.e., [TAR1-5-19]-Cys-maleimide-PEG×2, wherein the maleimides are linked together in the dimer) has an affinity in the TNF receptor assay of ˜1-3 nM (data not shown). Alternatively the dimer can also be produced using TMEA (Tris[2-maleimidoethyl]amine) (Pierce Biotechnology) or other bi-functional linkers.

As another alternative, one can produce the disulphide-linked dimer using a chemical coupling procedure using 2,2′-dithiodipyridine (Sigma Aldrich) and the reduced monomer. Addition of a polypeptide linker or hinge to the C-terminus of the dAb. A small linker, either (Gly₄Ser)ₙ where n=1 to 10, e.g., 1, 2, 3, 4, 5, 6 or 7, an immunoglobulin (e.g., IgG) hinge region or random peptide sequence (e.g., selected from a library of random peptide sequences) can be engineered between the dAb and the terminal cysteine residue. This could then be used to make dimers as described herein above.

### Example 5

### Additional Studies on Single Immunoglobulin Variable Domain Homodimers

Dimerization was investigated where V_{H} and V_{κ} homodimers were created in a dAb-linker-dAb format using flexible polypeptide linkers. Vectors were created in the dAb linker-dAb format containing glycine-serine linkers of different lengths 3U:(Gly₄Ser)₃, 5U:(Gly₄Ser)₅, 7U:(Gly₄Ser)₇. Dimer libraries were created using guiding dAbs upstream of the linker: TAR1-5 (V_{K}), TAR1-27 (V_{K}), TAR2 (V_{H}) or TAR1h-6 (V_{K}; also referred to as DOM1h-6) and a library of corresponding second dAbs after the linker. Using this method, novel dimeric dAbs were selected. The effect of dimerization on antigen binding was determined by ELISA and BIAcore studies and in the cell and receptor assays. Dimerization of both TAR1-5 and TAR1-27 resulted in significant improvement in binding affinity and neutralisation levels.

### Methods

A. Library Generation

1. Vectors

pEDA3U, pEDA5U and pEDA7U vectors were designed to introduce the different linker lengths compatible with the dAb-linker-dAb format. For pEDA3U, sense and anti-sense 73-base pair oligo linkers were annealed using a slow annealing program (95° C.-5 mins, 80° C.-10 mins, 70° C.-15 mins, 56° C.-15 mins, 42° C. until use) in buffer containing 0.1MNaCl, 10 mM Tris-HCl pH7.4 and cloned using the Xho1 and Not1 restriction sites. The linkers encompassed 3 (Gly₄Ser) units and a stuffer region housed between Sal1 and Not1 cloning sites. In order to reduce the possibility of monomeric dAbs being selected for by phage display, the stuffer region was designed to include 3 stop codons, a Sac1 restriction site and a frame shift mutation to put the region out of frame when no second dAb was present. For pEDA5U and 7U due to the length of the linkers required, overlapping oligo-linkers were designed for each vector, annealed and elongated using Klenow. The fragment was then purified and digested using the appropriate enzymes before cloning using the Xho1 and Not1 restriction sites.

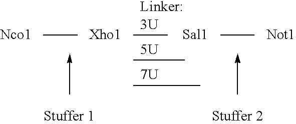

2. Library Preparation

The N-terminal V gene corresponding to the guiding dAb was cloned upstream of the linker using Nco1 and Xho1 restriction sites. V_{H} genes have existing compatible sites, however cloning V_{κ} genes required the introduction of suitable restriction sites. This was achieved by using modifying PCR primers (VK-DLIBF: 5′ CGGCCATGGCGTCAACGGACAT (SEQ ID NO: 173); VKXho1R: 5′ ATGTGCGCTCGAGCGTTTGATTT 3′ (SEQ ID NO: 174)) in 30 cycles of PCR amplification using a 2:1 mixture of SuperTaq (HTBiotechnology Ltd) and pfu turbo (Stratagene). This maintained the Nco1 site at the 5′ end while destroying the adjacent Sal1 site and introduced the Xho1 site at the 3′ end. 5 guiding dAbs were cloned into each of the 3 dimer vectors: TAR1-5 (V_{κ}), TAR1-27 (V_{κ}), TAR2 (V_{H}), TAR2h-6 (V_{κ}; also referred to as DOM1h-6) and TAR2h-7 (V_{κ}; also referred to as DOM1h-7). All constructs were verified by sequence analysis.

Having cloned the guiding dAbs upstream of the linker in each of the vectors (pEDA3U, 5U and 7U): TAR1-5 (V_{κ}), TAR1-27 (V_{κ}), TAR2 (V_{H}) or TAR2h-6 (V_{κ}) a library of corresponding second dAbs were cloned after the linker. To achieve this, the complimentary dAb libraries were PCR amplified from phage recovered from round 1 selections of either a V_{κ} library against TNF-α (at approximately 1×10⁶ diversity after round 1) when TAR1-5 or TAR1-27 are the guiding dAbs, or a V_{H} or V_{κ} library against p55 TNFR (both at approximately 1×10⁵ diversity after round 1) when TAR2 or TAR2h 6 respectively are the guiding dAbs. For V_{κ} libraries PCR amplification was conducted using primers in 30 cycles of PCR amplification using a 2:1 mixture of SuperTaq and pfu turbo. V_{H} libraries were PCR amplified using primers in order to introduce a Sal1 restriction site at the 5′ end of the gene. The dAb library PCRs were digested with the appropriate restriction enzymes, ligated into the corresponding vectors downstream of the linker, using Sal1/Not1 restriction sites and electroporated into freshly prepared competent TG1 cells.

The titres achieved for each library are as follows:
TAR1-5: pEDA3U=4×10⁸, pEDA5U=8×10⁷, pEDA7U=1×10⁸
TAR1-27: pEDA3U=6.2×10⁸, pEDA5U=1×10⁸, pEDA7U=1×10⁹
TAR2: pEDA3U=4×10⁷, pEDA5U=2×10⁸, pEDA7U=8×10⁷
TAR2h-6: pEDA3U=7.4×10⁸, pEDA5U=1.2×10⁸, pEDA7U=2.2×10⁸

B. Selections

1. TNF-α

Selections were conducted using human TNFα passively coated on immunotubes. The resulting anti-TNF-α Abs are referred using the nomenclature prefix “TAR1.” Briefly, Immunotubes are coated overnight with 1-4 mls of the required antigen. The immunotubes were then washed 3 times with PBS and blocked with 2% milk powder in PBS for 1-2 hrs and washed a further 3 times with PBS. The phage solution is diluted in 2% milk powder in PBS and incubated at room temperature for 2 hrs. The tubes are then washed with PBS and the phage eluted with 1 mg/ml trypsin-PBS. Three selection strategies were investigated for the TAR1-5 dimer libraries. The first round selections were carried out in immunotubes using human TNFα coated at 1 μg/ml or 20 μg/ml with 20 washes in PBS 0.1% Tween. TG1 cells are infected with the eluted phage and the titres are determined (e.g., Marks et al J Mol Biol. 1991 Dec. 5; 222(3):581-97, Richmann et al Biochemistry. 1993 Aug. 31; 32(34):8848-55).

The titres recovered were:
pEDA3U=2.8×10⁷ (1 μg/ml TNF) 1.5×10⁸ (20 μg/ml TNF),
pEDA5U=1.8×10⁷ (1 μg/ml TNF), 1.6×10⁸ (20 μg/ml TNF)
pEDA7U=8×10⁶ (1 μg/ml TNF), 7×10⁷ (20 μg/ml TNF).

The second round selections were carried out using 3 different methods:
1. In immunotubes, 20 washes with overnight incubation followed by a further 10 washes.
2. In immunotubes, 20 washes followed by 1 hr incubation at RT in wash buffer with (1 μg/ml TNF-α) and 10 further washes.
3. Selection on streptavidin beads using 33 pmoles biotinylated human TNFα. Single clones from round 2 selections were picked into 96 well plates and crude supernatant preps were made in 2 ml 96 well plate format.

TABLE 2

| | Round 1 TNF- | | | |
|---|---|---|---|---|
| | α immunotube | Round 2 | Round 2 | Round 2 |
| | coating | selection | selection | selection |
| | concentration | method 1 | method 2 | method 3 |
| pEDA3U | 1 μg/ml | 1 × 10⁹ | 1.8 × 10⁹ | 2.4 × 10¹⁰ |
| pEDA3U | 20 μg/ml | 6 × 10⁹ | 1.8 × 10¹⁰ | 8.5 × 10¹⁰ |
| pEDA5U | 1 μg/ml | 9 × 10⁸ | 1.4 × 10⁹ | 2.8 × 10¹⁰ |
| pEDA5U | 20 μg/ml | 9.5 × 10⁹ | 8.5 × 10⁹ | 2.8 × 10¹⁰ |
| pEDA7U | 1 μg/ml | 7.8 × 10⁸ | 1.6 × 10⁸ | 4 × 10¹⁰ |
| pEDA7U | 20 μg/ml | 1 × 10¹⁰ | 8 × 10⁹ | 1.5 × 10¹⁰ |

For TAR1-27, selections were carried out as described previously with the following modifications. The first round selections were carried out in immunotubes using human TNF-α coated at 1 μg/ml or 20 μg/ml with 20 washes in PBS 0.1% Tween. The second round selections were carried out in immunotubes using 20 washes with overnight incubation followed by a further 20 washes. Single clones from round 2 selections were picked into 96 well plates and crude supernatant preps were made in 2 ml 96 well plate format.

TAR1-27 titres are as follows:

TABLE 3

| | TNF-α | | |
|---|---|---|---|
| | immunotube | | |
| | coating conc | Round 1 | Round 2 |
| pEDA3U | 1 μg/ml | 4 × 10⁹ | 6 × 10⁹ |
| pEDA3U | 20 μg/ml | 5 × 10⁹ | 4.4 × 10¹⁰ |
| pEDA5U | 1 μg/ml | 1.5 × 10⁹ | 1.9 × 10¹⁰ |
| pEDA5U | 20 μg/ml | 3.4 × 10⁹ | 3.5 × 10¹⁰ |
| pEDA7U | 1 μg/ml | 2.6 × 10⁹ | 5 × 10⁹ |
| pEDA7U | 20 μg/ml | 7 × 10⁹ | 1.4 × 10¹⁰ |

2. p55 TNFR

Selections were conducted essentially as described for the anti-TNF binders, using p55 TNFR as the target antigen. 3 rounds of selections were carried out in immunotubes using either 1 μg/ml p55 TNFR or 10 μg/ml p55 TNFR with 20 washes in PBS 0.1% Tween with overnight incubation followed by a further 20 washes. Single clones from round 2 and 3 selections were picked into 96 well plates and crude supernatant preps were made in 2 ml 96 well plate format. Resulting anti-p55 TNFR dAbs are referred to using the nomenclature prefix “TAR2.”

TAR2 titres are as follows:

TABLE 4

| | Round 1 p55 | | | |
|---|---|---|---|---|
| | TNFR | | | |
| | immunotube | | | |
| | coating | | | |
| | concentration | Round 1 | Round 2 | Round 3 |
| pEDA3U | 1 μg/ml | 2.4 × 10⁶ | 1.2 × 10⁷ | 1.9 × 10⁹ |
| pEDA3U | 10 μg/ml | 3.1 × 10⁷ | 7 × 10⁷ | 1 × 10⁹ |
| pEDA5U | 1 μg/ml | 2.5 × 10⁶ | 1.1 × 10⁷ | 5.7 × 10⁸ |
| pEDA5U | 10 μg/ml | 3.7 × 10⁷ | 2.3 × 10⁸ | 2.9 × 10⁹ |
| pEDA7U | 1 μg/ml | 1.3 × 10⁶ | 1.3 × 10⁷ | 1.4 × 10⁹ |
| pEDA7U | 10 μg/ml | 1.6 × 10⁷ | 1.9 × 10⁷ | 3 × 10¹⁰ |

C. Screening

Single clones from round 2 or 3 selections were picked from each of the 3U, 5U and 7U libraries from the different selections methods, where appropriate. Clones were grown in 2×TY with 100 μg/ml ampicillin and 1% glucose overnight at 37° C. A 1/100 dilution of this culture was inoculated into 2 mls of 2×TY with 100 μg/ml ampicillin and 0.1% glucose in 2 ml, 96 well plate format and grown at 37° C. shaking until OD₆₀₀ was approximately 0.9. The culture was then induced with 1 mM IPTG overnight at 30° C. The supernatants were clarified by centrifugation at 4000 rpm for 15 mins in a Sorval plate centrifuge. The supernatant preps were used for initial screening.

1. ELISA

Binding activity of dimeric recombinant proteins was compared to monomer by Protein A/L ELISA or by antigen ELISA. Briefly, a 96 well plate is coated with antigen or Protein A/L overnight at 4° C. The plate washed with 0.05% Tween-PBS, blocked for 2 hrs with 2% Tween-PBS. The sample is added to the plate incubated for 1 hr at room temperature. The plate is washed and incubated with the secondary reagent for 1 hr at room temperature. The plate is washed and developed with TMB substrate. Protein A/L-HRP or India-HRP was used as a secondary reagent. For antigen ELISAs, the antigen concentrations used were 1 μg/ml in PBS for TNF-α and p55 TNFR. Due to the presence of the guiding dAb in most cases dimers gave a positive ELISA signal; therefore off rate determination was examined by BIAcore (SPR) analysis.

2. BIAcore (SPR) Analysis:

BIAcore analysis was conducted for TAR1-5 and TAR2 clones. For screening, TNF-α was coupled to a CM5 chip at high density (approximately 10000 RUs). 50 μl of TNF-α (50 μg/ml) was coupled to the chip at 5 μl/min in acetate buffer—pH5.5. Regeneration of the chip following analysis using the standard methods is not possible due to the instability of TNF-α therefore after each sample was analysed, the chip was washed for 10 mins with buffer.

For TAR1-5, clone supernatants from the round 2 selection were screened by BIAcore.

48 clones were screened from each of the 3U, 5U and 7U libraries obtained using the following selection methods:

R1: 1 μg/ml human TNFα immunotube, R2 1 μg/ml human TNFα immunotube, overnight wash.
R1: 20 μg/ml human TNFα immunotube, R2 20 μg/ml human TNFα immunotube, overnight wash.
R1: 1 μg/ml human TNFα immunotube, R2 33 pmoles biotinylated human TNFα on beads.
R1: 20 μg/ml human TNFα immunotube, R2 33 pmoles biotinylated human TNFα beads.

For screening, p55 TNFR (antigen previously referred to as DOM1, but for consistency referred to herein as p55 TNFR; as noted, resulting anti-p55 dAbs are referred to using the prefix “TAR2”) was coupled to a CM5 chip at high density (approximately 4000 RUs). 100 μl of p55 TNFR (10 μg/ml) was coupled to the chip at 5 μl/min in acetate buffer—pH5.5. Standard regeneration conditions were examined (glycine pH2 or pH3) but in each case antigen was removed from the surface of the chip, as with TNF-α; therefore, after each sample was analysed, the chip was washed for 10 mins with buffer.

For TAR2, clones supernatants from the round 2 selection were screened.

48 clones were screened from each of the 3U, 5U and 7U libraries, using the following selection methods:
R1: 1 μg/ml p55 TNFR immunotube, R2 1 μg/ml p55 TNFR immunotube, overnight wash.
R1: 10 μg/ml p55 TNFR immunotube, R2 10 μg/ml p55 TNFR immunotube, overnight wash.

3. Receptor and Cell Assays

The ability of the dimers to neutralize in the receptor assay was evaluated. Anti-TNF single immunoglobulin variable domains (“anti-TNF dAbs”) were tested for the ability to neutralize the cytotoxic activity of TNF on mouse L929 fibroblasts (Evans, T. (2000) Molecular Biotechnology 15, 243-248). Briefly, L929 cells plated in microtiter plates were incubated overnight with anti-TNF dAbs, 100 pg/ml TNF-α and 1 mg/ml actinomycin D (Sigma, Poole, UK). Cell viability was measured by reading absorbance at 490 nm following an incubation with [3-(4,5-dimethylthiazol-2-yl)-5-(3-carboxymethoxyphenyl)-2-(4-sulfophenyl)-2H-tetrazolium (Promega, Madison, USA). Anti-TNF dAb activity led to a decrease in TNF cytotoxicity and therefore an increase in absorbance compared with the TNF only control.

As a preferred approach when evaluating the effect of a single immunoglobulin variable domain polypeptide on the p55 TNF-α receptor, the following HeLa cell assay based on the induction of IL-8 secretion by TNF in HeLa cells is used (method is adapted from that of Akeson, L. et al (1996) Journal of Biological Chemistry 271, 30517-30523, describing the induction of IL-8 by IL-1 in HUVEC; here we look at induction by human TNF alpha and we use HeLa cells instead of the HUVEC cell line). Briefly, HeLa cells plated in microtitre plates were incubated overnight with dAb and 300 pg/ml TNF. Following incubation, the supernatant was aspirated off the cells and the IL-8 concentration was measured via a sandwich ELISA (R&D Systems). Anti-TNFR1 dAb activity led to a decrease in IL-8 secretion into the supernatant compared with the TNF only control.

Anti-TNF dAbs have also been tested for the ability to inhibit the binding of TNF to recombinant TNF receptor 1 (p55) as follows. Briefly, Maxisorp plates were incubated overnight with 30 mg/ml anti-human Fc mouse monoclonal antibody (Zymed, San Francisco, USA). The wells were washed with phosphate buffered saline (PBS) containing 0.05% Tween-20 and then blocked with 1% BSA in PBS before being incubated with 100 ng/ml TNF receptor 1 Fc fusion protein (R&D Systems, Minneapolis, USA). Anti-TNF dAb was mixed with TNF which was added to the washed wells at a final concentration of 10 ng/ml. TNF binding was detected with 0.2 mg/ml biotinylated anti-TNF antibody (HyCult biotechnology, Uben, Netherlands) followed by 1 in 500 dilution of horseradish peroxidase labelled streptavidin (Amersham Biosciences, UK) and incubation with TMB substrate (KPL, Gaithersburg, Md.). The reaction was stopped by the addition of HCl and the absorbance was read at 450 nm. Anti-TNF dAb inhibitory activity led to a decrease in TNF binding and therefore to a decrease in absorbance compared with the TNF only control.

In the initial screen, supernatants prepared for BIAcore analysis, described above, were also used in the receptor assay. Further analysis of selected dimers was also conducted in the receptor and cell assays using purified proteins.

D. Sequence Analysis

Dimers that proved to have interesting properties in the BIAcore and the receptor assay screens were sequenced. Sequences are detailed in Table 5.

E. Formatting

1. TAR1-5-19 Dimers

The TAR1-5 dimers that were shown to have good neutralization properties were re-formatted and analysed in the cell and receptor assays. The TAR1-5 guiding dAb was substituted with the affinity matured clone TAR1-5-19. To achieve this, TAR1-5 was cloned out of the individual dimer pair and substituted with TAR1-5-19 that had been amplified by PCR. In addition, TAR1-5-19 homodimers were also constructed in the 3U, 5U and 7U vectors. The N terminal copy of the gene was amplified by PCR and cloned as described above and the C-terminal gene fragment was cloned using existing Sal1 and Not1 restriction sites.

2. Mutagenesis

The amber stop codon present in dAb2, one of the C-terminal dAbs in the TAR1-5 dimer pairs was mutated to a glutamine by site-directed mutagenesis.

3. Fabs

The dimers containing TAR 1-5 or TAR1-5-19 were re-formatted into Fab expression vectors. dAbs were cloned into expression vectors containing either the C_{κ} or C_{H} genes using Sfi1 and Not1 restriction sites and verified by sequence analysis. The C_{κ} vector is derived from a pUC based ampicillin resistant vector and the C_{H} vector is derived from a pACYC chloramphenicol resistant vector. For Fab expression the dAb-C_{H} and dAb-C_{κ} constructs were co-transformed into HB2151 cells and grown in 2×TY containing 0.1% glucose, 100 μg/ml ampicillin and 10 μg/ml chloramphenicol.

4. Hinge Dimerization

Dimerization of dAbs via cystine bond formation was examined. A short sequence of amino acids EPKSGDKTHTCPPCP (SEQ ID NO: 175) a modified form of the human IgGC1 hinge, was engineered at the C terminal region on the dAb. An oligo linker encoding this sequence was synthesized and annealed, as described previously. The linker was cloned into the pEDA vector containing TAR1-5-19 using Xho1 and Not1 restriction sites. Dimerization occurs in situ in the periplasm.

F. Expression and Purification

1. Expression

Supernatants were prepared in the 2 ml, 96-well plate format for the initial screening as described previously. Following the initial screening process selected dimers were analysed further. Dimer constructs were expressed in TOP10F′ or HB2151 cells as supernatants. Briefly, an individual colony from a freshly streaked plate was grown overnight at 37° C. in 2×TY with 100 μg/ml ampicillin and 1% glucose. A 1/100 dilution of this culture was inoculated into 2×TY with 100 μg/ml ampicillin and 0.1% glucose and grown at 37° C. shaking until OD600 was approximately 0.9. The culture was then induced with 1 mM IPTG overnight at 30° C. The cells were removed by centrifugation and the supernatant purified with protein A or L agarose.

Fab and cysteine hinge dimers were expressed as periplasmic proteins in HB2152 cells. A 1/100 dilution of an overnight culture was inoculated into 2×TY with 0.1% glucose and the appropriate antibiotics and grown at 30° C. shaking until OD600 was approximately 0.9. The culture was then induced with 1 mM IPTG for 3-4 hours at 25° C. The cells were harvested by centrifugation and the pellet resuspended in periplasmic preparation buffer (30 mM Tris-HCl pH8.0, 1 mM EDTA, 20% sucrose). Following centrifugation the supernatant was retained and the pellet resuspended in 5 mM MgSO₄. The supernatant was harvested again by centrifugation, pooled and purified.

2. Protein A/L Purification

Optimization of the purification of dimer proteins from Protein L agarose (Affitech, Norway) or Protein A agarose (Sigma, UK) was examined. Protein was eluted by batch or by column elution using a peristaltic pump. Three buffers were examined 0.1M Phosphate-citrate buffer pH2.6, 0.2M Glycine pH2.5 and 0.1M Glycine pH2.5. The optimal condition was determined to be under peristaltic pump conditions using 0.1M Glycine pH2.5 over 10 column volumes. Purification from protein A was conducted using peristaltic pump conditions and 0.1M Glycine pH2.5.

3. FPLC Purification

Further purification was carried out by FPLC analysis on an AKTA Explorer 100 system (Amersham Biosciences Ltd). TAR1-5 and TAR1-5-19 dimers were fractionated by cation exchange chromatography (1 ml Resource S—Amersham Biosciences Ltd) eluted with a 0-1M NaCl gradient in 50 mM acetate buffer pH4. Hinge dimers were purified by ion exchange (1 ml Resource Q Amersham Biosciences Ltd) eluted with a 0-1M NaCl gradient in 25 mM Tris HCl pH 8.0. Fabs were purified by size exclusion chromatography using a superose 12 (Amersham Biosciences Ltd) column run at a flow rate of 0.5 ml/min in PBS with 0.05% tween. Following purification, samples were concentrated using VIVASPIN™ 5K cut off concentrators (Vivascience Ltd).

### Results

A. TAR1-5 Dimers

6×96 clones were picked from the round 2 selection encompassing all the libraries and selection conditions. Supernatant preps were made and assayed by antigen and Protein L ELISA, BIAcore and in the receptor assays. In ELISAs, positive binding clones were identified from each selection method and were distributed between 3U, 5U and 7U libraries. However, as the guiding dAb is always present it was not possible to discriminate between high and low affinity binders by this method; therefore BIAcore SPR analysis was conducted.

BIAcore analysis was conducted using the 2 ml supernatants. BIAcore analysis revealed that the dimer K_{off} rates were vastly improved compared to monomeric TAR1-5. Monomer K_{off} rate was in the range of 10⁻¹M compared with dimer K_{off} rates which were in the range of 10⁻³-10⁻⁴M. 16 clones that appeared to have very slow off rates were selected, these came from the 3U, 5U and 7U libraries and were sequenced. In addition the supernatants were analysed for the ability to neutralise human TNFα in the receptor assay.

6 lead clones (d1-d6 below) that neutralised in these assays have been sequenced. The results shows that out of the 6 clones obtained there are only 3 different second dAbs (dAb1, dAb2 and dAb3) however where the second dAb is found more than once they are linked with different length linkers.

TAR1-5d1: 3U linker 2^{nd} dAb=dAb1−1 μg/ml Ag immunotube overnight wash
TAR1-5d2: 3U linker 2^{nd} dAb=dAb2−1 μg/ml Ag immunotube overnight wash
TAR1-5d3: 5U linker 2^{nd} dAb=dAb2−1 μg/ml Ag immunotube overnight wash
TAR1-5d4: 5U linker 2^{nd} dAb=dAb3−20 μg/ml Ag immunotube overnight wash
TAR1-5d5: 5U linker 2^{nd} dAb=dAb1−20 μg/ml Ag immunotube overnight wash
TAR1-5d6: 7U linker 2^{nd} dAb=dAb1−R1:1 μg/ml Ag immunotube overnight wash, R2:beads

The 6 lead clones were examined further. Protein was produced from the periplasm and supernatant, purified with protein L agarose and examined in the cell and receptor assays. The levels of neutralisation were variable (Table 5). The optimal conditions for protein preparation were determined. Protein produced from HB2151 cells as supernatants gave the highest yield (approximately 10 mgs/L of culture). The supernatants were incubated with protein L agarose for 2 hrs at room temperature or overnight at 4° C. The beads were washed with PBS/NaCl and packed onto an FPLC column using a peristaltic pump. The beads were washed with 10 column volumes of PBS/NaCl and eluted with 0.1M glycine pH2.5. In general, dimeric protein is eluted after the monomer.

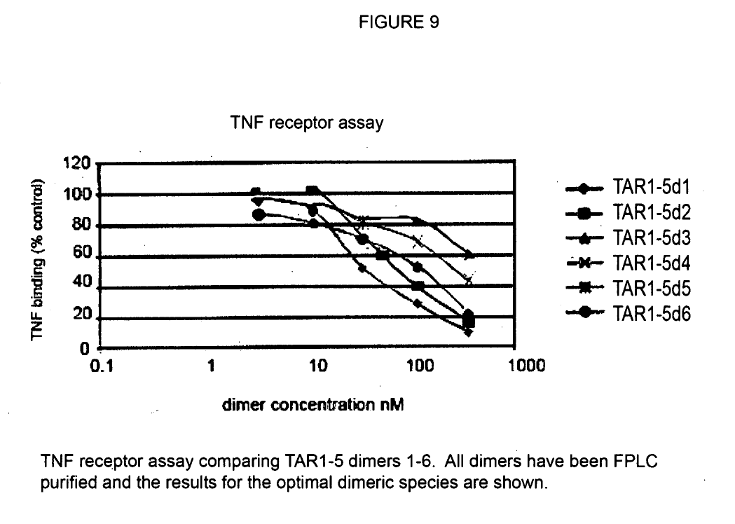

TAR1-5d1-6 dimers were purified by FPLC. Three species were obtained, by FPLC purification and were identified by SDS PAGE. One species corresponds to monomer and the other two species correspond to dimers of different sizes. The larger of the two species is possibly due to the presence of C terminal tags. These proteins were examined in the receptor assay. The data presented in Table 5 represents the optimum results obtained from the two dimeric species (FIG. 9)

The three second dAbs from the dimer pairs (i.e., dAb1, dAb2 and dAb3) were cloned as monomers and examined by ELISA and in the cell and receptor assay. All three dAbs bind specifically to TNF by antigen ELISA and do not cross react with plastic or BSA. As monomers, none of the dAbs neutralise in the cell or receptor assays.

B. TAR1-5-19 Dimers

TAR1-5-19 was substituted for TAR1-5 in the 6 lead clones. Analysis of all TAR1-5-19 dimers in the cell and receptor assays was conducted using total protein (protein L purified only) unless otherwise stated (Table 6). TAR1-5-19d4 and TAR1-5-19d3 have the best ND₅₀ (˜5 nM) in the cell assay—this is consistent with the receptor assay results and is an improvement over TAR1-5-19 monomer (ND₅₀˜30 nM). Although purified TAR1-5 dimers give variable results in the receptor and cell assays TAR1-5-19 dimers were more consistent. Variability was shown when using different elution buffers during the protein purification. Elution using 0.1M Phosphate-citrate buffer pH2.6 or 0.2M Glycine pH2.5 although removing all protein from the protein L agarose in most cases rendered it less functional.

TAR1-5-19d4 was expressed in the fermenter and purified on cation exchange FPLC to yield a completely pure dimer. As with TAR1-5d4, three species were obtained by FPLC purification corresponding to one monomer and two dimer species

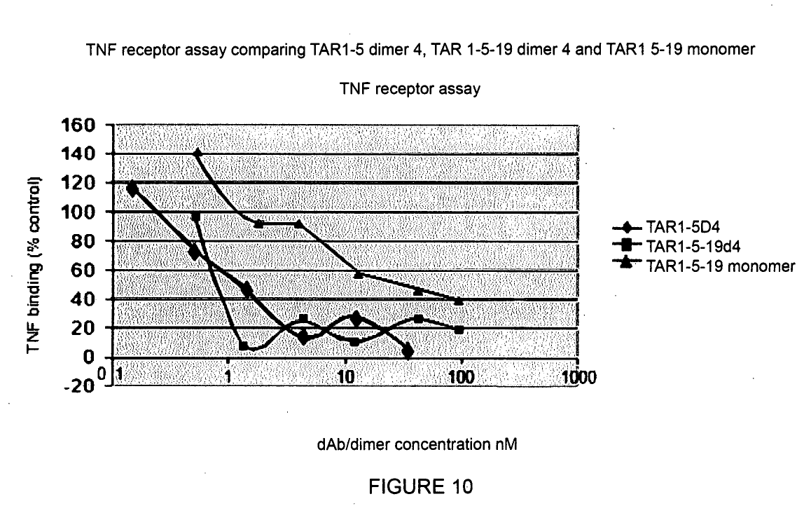

The TAR1-5-19d4 dimer was amino acid analyzed. TAR1-5-19 monomer and TAR1-5-19d4 were then examined in the receptor assay and the resulting IC₅₀ for monomer was 30 nM and for dimer was 8 nM. The results of the receptor assay comparing TAR1-5-19 monomer, TAR1-5-19d4 and TAR1-5d4 is shown in FIG. 10.

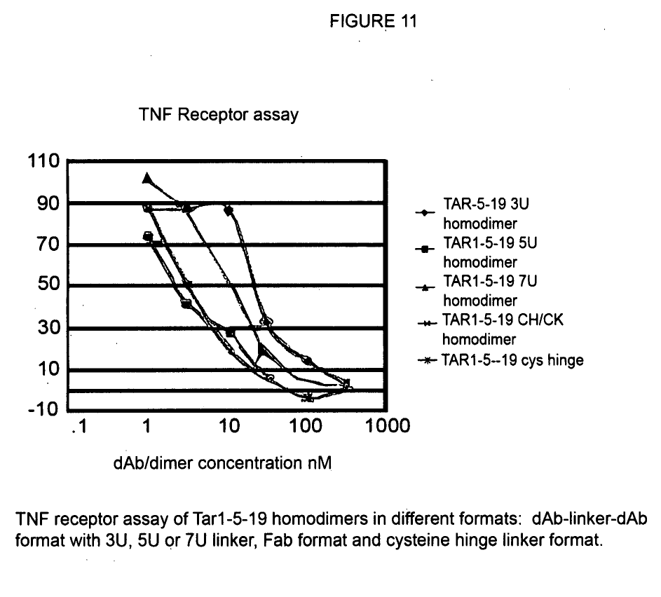

TAR1-5-19 homodimers were made in the 3U, 5U and 7U vectors, expressed and purified on Protein L. The proteins were examined in the cell and receptor assays and the resulting IC₅₀s (for receptor assay) and ND₅₀s (for cell assay) were determined (Table 7, FIG. 11).

C. Fabs

TAR1-5 and TAR1-5-19 dimers were also cloned into Fab format, expressed and purified on protein L agarose. Fabs were assessed in the receptor assays (Table 8). The results showed that for both TAR1-5-19 and TAR1-5 dimers the neutralization levels were similar to the original Gly₄Ser linker dimers from which they were derived. A TAR1-5-19 Fab where TAR1-5-19 was displayed on both C_{H} and V_{κ} was expressed, protein L purified and assessed in the receptor assay. The resulting IC₅₀ was approximately 1 nM.

D. TAR1-27 Dimers

3×96 clones were picked from the round 2 selection encompassing all the libraries and selection conditions. 2 ml supernatant preps were made for analysis in ELISA and bioassays. Antigen ELISA gave 71 positive clones. The receptor assay of crude supernatants yielded 42 clones with inhibitory properties (TNF binding 0-60%). In the majority of cases inhibitory properties correlated with a strong ELISA signal. 42 clones were sequenced, 39 of these have unique second dAb sequences. The 12 dimers that gave the best inhibitory properties were analysed further.

The 12 neutralizing clones were expressed as 200 ml supernatant preps and purified on protein L. These were assessed by protein L and antigen ELISA, BIAcore and in the receptor assay. Strong positive ELISA signals were obtained in all cases. BIAcore analysis revealed all clones to have fast on and off rates. The off rates were improved compared to monomeric TAR1-27, however the off rate of TAR1-27 dimers was faster (K_{off} is approximately in the range of 10⁻¹ and 10⁻²M) than the TAR1-5 dimers examined previously (K_{off} is approximately in the range of 10⁻³-10⁻⁴M). The stability of the purified dimers was questioned and therefore in order to improve stability, the addition on 5% glycerol, 0.5% Triton X100 or 0.5% NP40 (Sigma) was included in the purification of 2 TAR1-27 dimers (d2 and d16). Addition of NP40 or Triton X100™ improved the yield of purified product approximately 2 fold. Both dimers were assessed in the receptor assay. TAR1-27d2 gave IC50 of ˜30 nM under all purification conditions. TAR1-27d16 showed no neutralisation effect when purified without the use of stabilising agents but gave an IC50 of ˜50 nM when purified under stabilising conditions.

E. TAR2 Dimers

3×96 clones were picked from the second round selections encompassing all the libraries and selection conditions. 2 ml supernatant preps were made for analysis. Protein A and antigen ELISAs were conducted for each plate. 30 interesting clones were identified as having good off-rates by BIAcore (K_{off} ranges between 10⁻²-10⁻³M). The clones were sequenced and 13 unique dimers were identified by sequence analysis.

F. Sequences

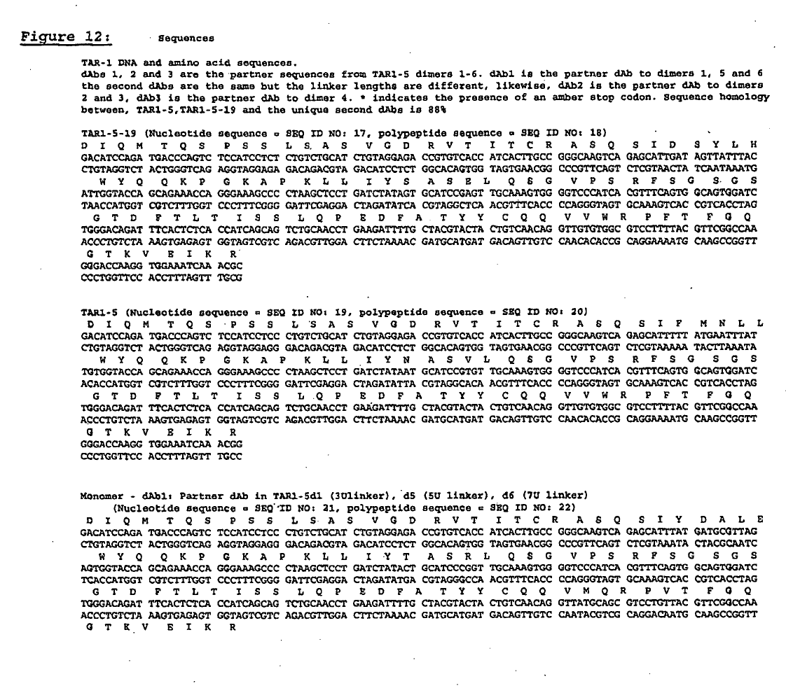

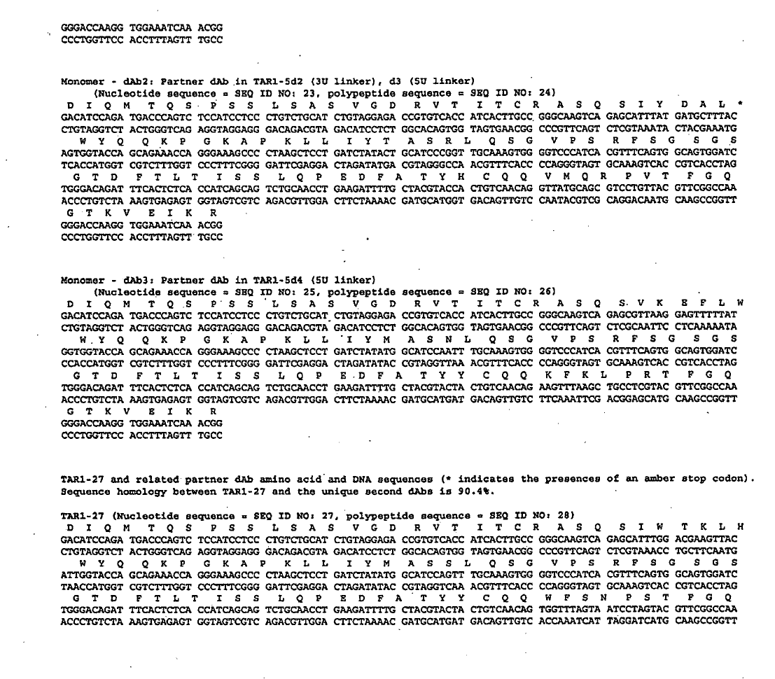

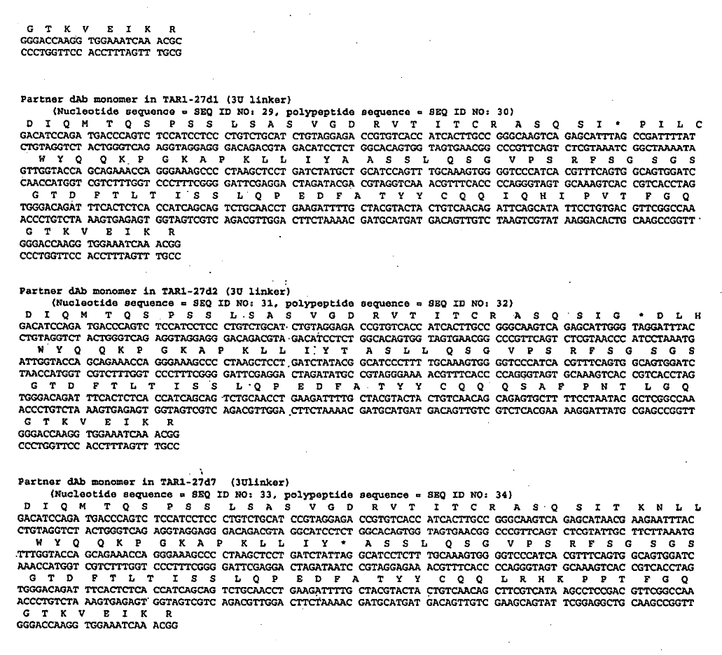

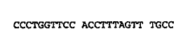

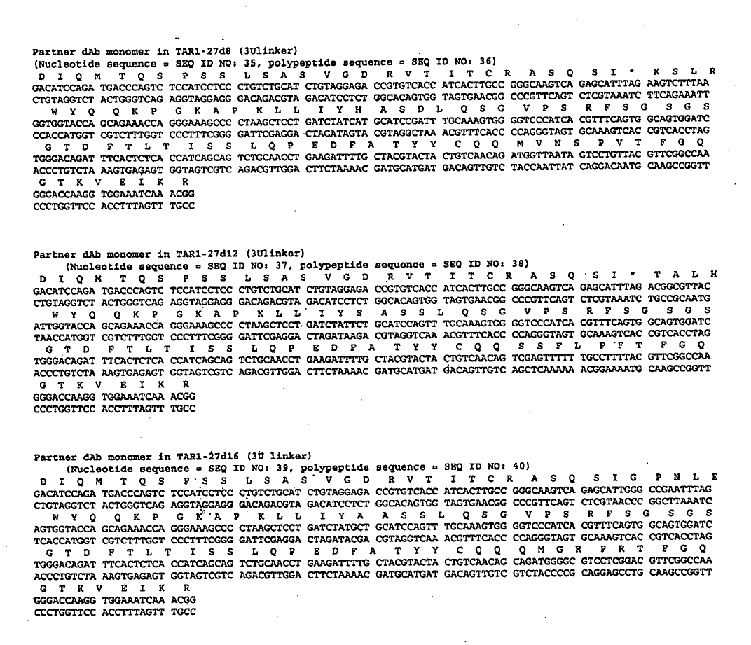

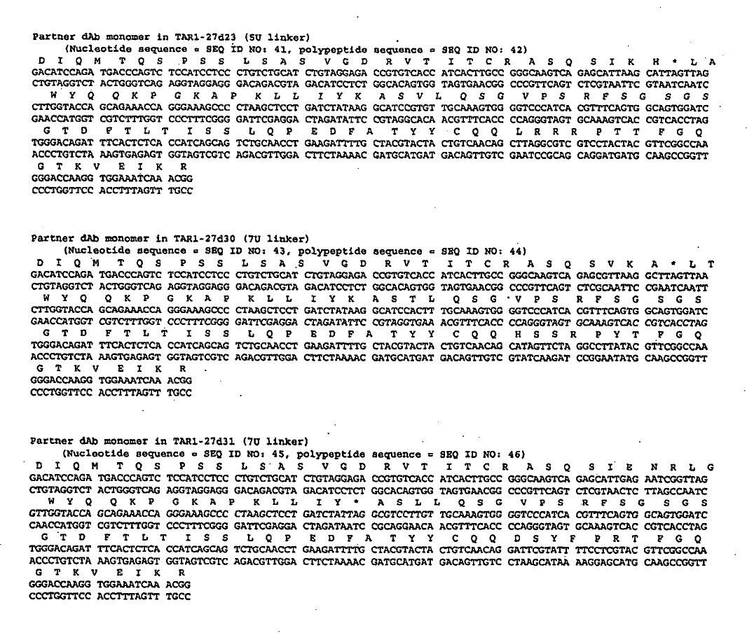

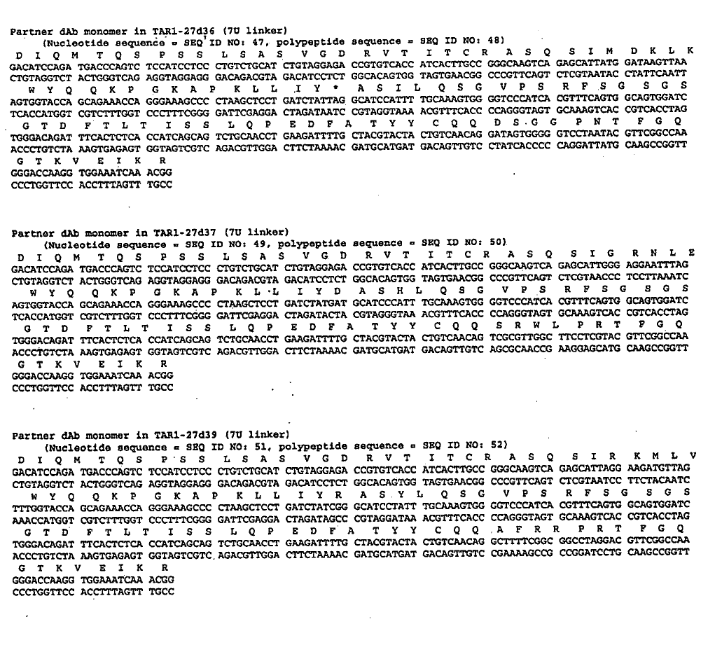

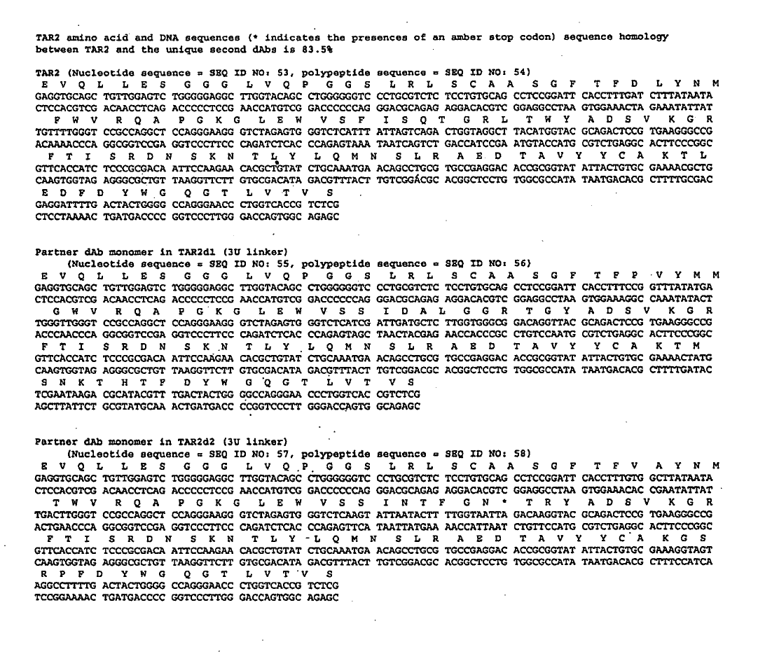

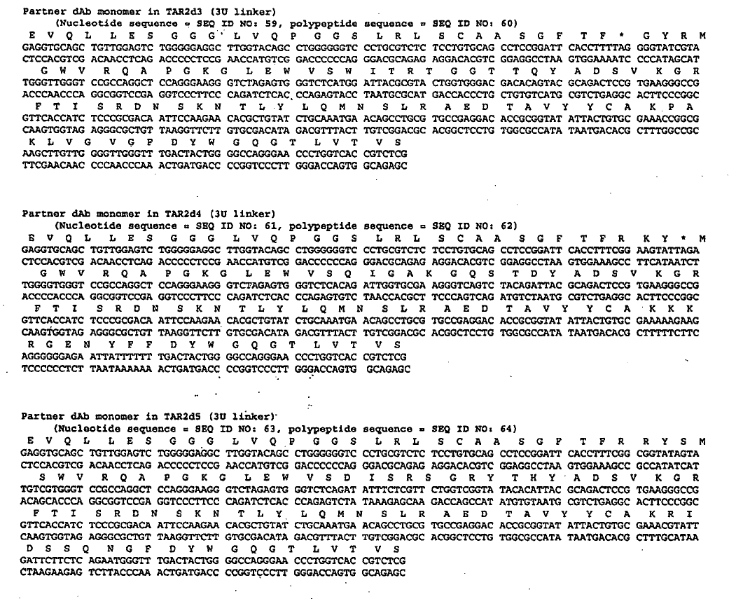

Nucleotide and amino acid sequences for dAbs described in this Example are provided in FIG. 12.

TABLE 5
TAR1-5 dimers

| | | | | Elution | Receptor/ |
|---|---|---|---|---|---|
| Dimer | Cell type | Purification | Protein Fraction | conditions | Cell assay |
| TAR1-5d1 | HB2151 | Protein L + | small dimeric | 0.1M glycine | RA ~30 nM |
| | | FPLC | species | pH2.5 | |
| TAR1-5d2 | HB2151 | Protein L + | small dimeric | 0.1M glycine | RA ~50 nM |
| | | FPLC | species | pH2.5 | |
| TAR1-5d3 | HB2151 | Protein L + | large dimeric | 0.1M glycine | RA ~300 nM |
| | | FPLC | species | pH2.5 | |
| TAR1-5d4 | HB2151 | Protein L + | small dimeric | 0.1M glycine | RA ~3 nM |
| | | FPLC | species | pH2.5 | |
| TAR1-5d5 | HB2151 | Protein L + | large dimeric | 0.1M glycine | RA ~200 nM |
| | | FPLC | species | pH2.5 | |
| TAR1-5d6 | HB2151 | Protein L + | Large dimeric | 0.1M glycine | RA ~100 nM |
| | | FPLC | species | pH2.5 | |

*note dimer 2 and dimer 3 have the same second dAb (called dAb2); however, they have different linker lengths (d2 = (Gly₄Ser)₃, d3 = (Gly₄Ser)₃). dAb1 is the partner dAb to dimers 1, 5 and 6. dAb3 is the partner dAb to dimer4. None of the partner dAbs neutralise alone. FPLC purification is by cation exchange unless otherwise stated. The optimal dimeric species for each dimer obtained by FPLC was determined in these assays.

TABLE 6
TAR1-5-19 dimers

| | Cell | | Protein | Elution | Receptor/Cell |
|---|---|---|---|---|---|
| Dimer | type | Purification | Fraction | conditions | assay |
| TAR1-5-19 d1 | TOP10F, | Protein L | Total protein | 0.1M glycine pH | RA ~15 nM |
| | | | | 2.0 | |
| TAR1-5-19 d2 | TOP10F, | Protein L | Total protein | 0.1M glycine pH | RA ~2 nM |
| (no stop codon) | | | | 2.0 + 0.05% NP40 | |
| TAR1-5-19d3 | TOP10F, | Protein L | Total protein | 0.1M glycine pH | RA ~8 nM |
| (no stop codon) | | | | 2.5 + 0.05% NP40 | |
| TAR1-5-19d4 | TOP10F, | Protein L + | FPLC purified | 0.1M glycine | RA ~2-5 nM |
| | | FPLC | fraction | pH2.0 | CA ~12 nM |
| TAR1-5-19d5 | TOP10F, | Protein L | Total protein | 0.1M glycine | RA ~8 nM |
| | | | | pH2.0 + NP40 | CA ~10 nM |
| TAR1-5-19 d6 | TOP10F, | Protein L | Total protein | 0.1M glycine pH | RA ~10 nM |
| | | | | 2.0 | |

TABLE 7
TAR1-5-19 homodimers

| | Cell | | Protein | Elution | Receptor/Cell |
|---|---|---|---|---|---|
| Dimer | type | Purification | Fraction | conditions | assay |
| TAR1-5-19 3U | HB2151 | Protein L | Total protein | 0.1M glycine | RA ~20 nM |
| homodimer | | | | pH2.5 | CA ~30 nM |
| TAR1-5-19 5U | HB2151 | Protein L | Total protein | 0.1M glycine | RA ~2 nM |
| homodimer | | | | pH2.5 | CA ~3 nM |
| TAR1-5-19 7U | HB2151 | Protein L | Total protein | 0.1M glycine | RA ~10 nM |
| homodimer | | | | pH2.5 | CA ~15 nM |
| TAR1-5-19 cys | HB2151 | Protein L + | FPLC purified | 0.1M glycine | RA ~2 nM |
| hinge | | FPLC | dimer fraction | pH2.5 | |
| TAR1-5- | HB2151 | Protein | Total protein | 0.1M glycine | RA ~1 nM |
| 19CH/TAR1- | | | | pH2.5 | |
| 5-19 CK | | | | | |

TABLE 8
TAR1-5/TAR1-5-19 Fabs

| | | | | Elution | Receptor/Cell |
|---|---|---|---|---|---|
| Dimer | Cell type | Purification | Protein Fraction | conditions | assay |
| TAR1-5CH/ | HB2151 | Protein L | Total protein | 0.1M citrate | RA ~90 nM |
| dAb1 CK | | | | pH2.6 | |
| TAR1-5CH/ | HB2151 | Protein L | Total protein | 0.1M glycine | RA ~30 nM |
| dAb2 CK | | | | pH2.5 | CA ~60 nM |
| dAb3CH/ | HB2151 | Protein L | Total protein | 0.1M citrate | RA ~100 nM |
| TAR1-5CK | | | | pH2.6 | |
| TAR1-5- | HB2151 | Protein L | Total protein | 0.1M glycine | RA ~6 nM |
| 19CH/ | | | | pH2.0 | |
| dAb1 CK | | | | | |
| dAb1 CH/ | HB2151 | Protein L | 0.1M glycine | Myc/flag | RA ~6 nM |
| TAR1-5-19CK | | | pH2.0 | | |
| TAR1-5- | HB2151 | Protein L | Total protein | 0.1M glycine | RA ~8 nM |
| 19CH/ | | | | pH2.0 | CA ~12 nM |
| dAb2 CK | | | | | |
| TAR1-5- | HB2151 | Protein L | Total protein | 0.1M glycine | RA ~3 nM |
| 19CH/ | | | | pH2.0 | |
| dAb3CK | | | | | |

### Example 6

### Formation of a Homotrimer of a TNF-α-Specific Single Immunoglobulin Variable Domain

For dAb trimerisation, cysteine-modified monomers isolated from the expression of TAR1-5-19CYS as described in Example 4 were reduced to yield free thiol, and then reacted with a trimeric maleimide molecule, to yield a chemically linked homotrimer.

Trimerization of TAR1-5-19CYS

2.5 ml of 100 μM TAR1-5-19CYS was reduce with 5 mM dithiothreitol and left at room temperature for 20 minutes. The sample was then buffer exchanged using a PD-10 column (Amersham Pharmacia). The column had been pre-equilibrated with 5 mM EDTA, 50 mM sodium phosphate pH 6.5, and the sample applied and eluted following the manufactures guidelines. The sample was placed on ice until needed. TMEA (Tris[2-maleimidoethyl]amine) was purchased from Pierce Biotechnology. A 20 mM stock solution of TMEA was made in 100% DMSO (dimethyl sulfoxide). It was found that a concentration of TMEA greater than 3:1 (molar ratio of dAb:TMEA) caused the rapid precipitation and cross-linking of the protein. Also the rate of precipitation and cross-linking was greater as the pH increased. Therefore using 100 μM reduced TAR1-5-19CYS, 25 μM TMEA was added to trimerize the protein and the reaction was allowed to proceed at room temperature for two hours. It was found that the addition of additives such as glycerol or ethylene glycol to 20% (v/v), significantly reduced the precipitation of the trimer as the coupling reaction proceeded. After coupling, SDS-PAGE analysis showed the presence of monomer, dimer and trimer in solution.

Purification of the Trimeric TAR1-5-19CYS

40 μL of 40% glacial acetic acid was added per mL of the TMEA-TAR1-5-19Cys reaction to reduce the pH to ˜4. The sample was then applied to a 1 mL Resource S cation exchange column (Amersham Pharmacia), which had been pre-equilibrated with 50 mM sodium acetate pH 4.0. The dimer and trimer were partially separated using a salt gradient of 340 to 450 mM Sodium chloride, 50 mM sodium acetate pH 4.0 over 30 column volumes. Fractions containing trimer only were identified using SDS-PAGE and then pooled and the pH increased to 8 by the addition of ⅕ volume of 1M Tris pH 8.0. To prevent precipitation of the trimer during concentration steps (using 5K cut off Vivaspin concentrators; Vivascience), 10% glycerol was added to the sample.

In Vitro Functional Binding Assay: TNF Receptor Assay and Cell Assay

The affinity of the trimer for human TNFα was determined using the TNF receptor and cell assay. IC₅₀ in the receptor assay was 0.3 nM; ND₅₀ in the cell assay was in the range of 3 to 10 nM (e.g., 3 nM).

Other Possible TAR1-5-19CYS Trimer Formats

TAR1-5-19CYS may also be formatted into a trimer using the following reagents:

PEG trimers and custom synthetic maleimide trimers. Nektar (Shearwater) offer a range of multi arm PEGs, which can be chemically modified at the terminal end of the PEG. Therefore using a PEG trimer with a maleimide functional group at the end of each arm would allow the trimerisation of the dAb in a manner similar to that outlined above using TMEA. The PEG may also have the advantage in increasing the solubility of the trimer thus preventing the problem of aggregation. Thus, one could produce a dAb trimer in which each dAb has a C-terminal cysteine that is linked to a maleimide functional group, the maleimide functional groups being linked to a PEG trimer.

Addition of a Polypeptide Linker or Hinge to the C-Terminus of the dAb

A small linker, either (Gly₄Ser)ₙ where n=1 to 10, e.g., 1, 2, 3, 4, 5, 6 or 7, an immunoglobulin (e.g., IgG) hinge region or random peptide sequence (e.g., selected from a library of random peptide sequences) could be engineered between the dAb and the terminal cysteine residue. When used to make multimers (e.g., dimers or trimers), this again would introduce a greater degree of flexibility and distance between the individual monomers, which may improve the binding characteristics to the target, e.g. a multisubunit target such as human TNF-α.

A summary of available data regarding concentration, affinity and functional properties of exemplary single immunoglobulin variable domain polypeptides described herein is provided in Table 9.

TABLE 9
Summary of affinities, concentrations and functional properties of dAbs

| | | | | | | ND50 for |
|---|---|---|---|---|---|---|
| | | Concn of | Equilibrium | | | cell based |
| | | samples made | dissocation constant | | | neutralisn |
| TARGET | dAb | to date | (Kd = Koff/Kon) | Koff | IC50 for ligand assay | assay |
| TNF-α | TAR1 | | 300 nM to 5pM | 5 × 10⁻¹ to 1 × 10⁻⁷ | 500 nM to 100pM | 500 nM to |
| | monomers | | (ie, 3 × 10⁻⁷ to | | | 50 pM |
| | | | 5 × 10⁻¹²), preferably | | | |
| | | | 50 nM to 20pM | | | |
| | TAR1 dimers | | As TAR1 monomer | As TAR1 | As TAR1 monomer | As TAR1 |
| | | | | monomer | | monomer |
| | TAR1 trimers | | As TAR1 monomer | As TAR1 | As TAR1 monomer | As TAR1 |
| | | | | monomer | | monomer |
| | TAR1-5 | | | | | |
| | TAR1-27 | | | | | |
| | TAR1-5-19 | 40 mg/ml | 30 nM | | | |
| | monomer | (mutant | | | | |
| | | A50P = | | | | |
| | | 40 mg/ml | | | | |
| | | also) | | | | |
| | TAR1-5-19 | TAR1-5- | | | With (Gly₄Ser)₃ linker = 20 nm | =30 nM |
| | homodimer | 193U = | | | | |
| | | 1 mg/ml | | | | |
| | | TAR1-5- | | | With (Gly₄Ser)₅ linker = 2 nm | |
| | | 195U = | | | | |
| | | 0.8 mg/ml | | | | |
| | | TAR1-5- | | | With (Gly₄Ser)₇ linker = 10 nm | |
| | | 197U = | | | | |
| | | 0.16 mg/ml | | | | |
| | | Fab format = | | | In Fab format = 1 nM | =3 nM |
| | | 0.14 mg/ml | | | | |
| | | | | | | =15 nM |
| | TAR1-5-19 | TAR1-5- | | | With (Gly₄Ser)ₙ linker | |
| | heterodimers | 19d1 = | | | | |
| | | 0.36 mg/ml | | | | |
| | | TAR1-5- | | | TAR1-5-19 d2 = 2 nM | |
| | | 19d2 = | | | | |
| | | 0.3 mg/ml | | | | |
| | | TAR1-5- | | | TAR1-5-19 d3 = 8 nM | |
| | | 19d3 = | | | | |
| | | 0.5 mg/ml | | | | |
| | | TAR1-5- | | | TAR1-5-19 d4 = 2-5 nM | =12 nM |
| | | 19d4 = | | | | |
| | | 0.53 mg/ml | | | | |
| | | TAR1-5- | | | TAR1-5-19 d5 = 8 nM | =10 nM |
| | | 19d5 = | | | | |
| | | 0.6 mg/ml | | | | |
| | | TAR1-5- | | | | |
| | | 19d6 = | | | | |
| | | 0.4 mg/ml | | | | |
| | | TAR1-5-19CH | | | In Fab format | |
| | | d1CK = | | | TAR1-5-19CH d1CK = 6 nM | |
| | | 0.41 mg/ml | | | | |
| | | TAR1-5-19CK | | | TAR1-5-19CK d1CH = 6 nM | |
| | | d1CH = | | | | |
| | | 0.32 mg/ml | | | | |
| | | TAR1-5-19CH | | | TAR1-5-19CH d2CK = 8 nM | |
| | | d2CK = | | | | |
| | | 0.25 mg/ml | | | | |
| | | TAR1-5-19CH | | | TAR1-5-19CH d3CK = 3 nM | |
| | | d3CK = | | | | |
| | | 0.2 mg/ml | | | | |
| | | | | | | =12 nM |
| | TAR1-5 | TAR1-5d1 = | | | With (Gly₄Ser)ₙ linker | |
| | heterodimers | 1 mg/ml | | | TAR1-5d1 = 30 nM | |
| | | TAR1-5d2 = | | | TAR1-5d2 = 50 nM | |
| | | 1 mg/ml | | | | |
| | | TAR1-5d3 = | | | TAR1-5d3 = 300 nM | |
| | | 1 mg/ml | | | | |
| | | TAR1-5d4 = | | | TAR1-5d4 = 3 nM | |
| | | 1 mg/ml | | | | |
| | | TAR1-5d5 = | | | TAR1-5d5 = 200 nM | |
| | | 0.5 mg/ml | | | | |
| | | TAR1-5d6 = | | | TAR1-5d6 = 100 nM | |
| | | 0.3 mg/ml | | | | |
| | | TAR1-5CH | | | In Fab format | |
| | | d2CK = | | | TAR1-5CH d2CK = 30 nM | =60 nM |
| | | 0.18 mg/ml | | | | |
| | | TAR1-5CK | | | TAR1-5CK d3CH = 100 nM | |
| | | d3CH = | | | | |
| | | 0.18 mg/ml | | | | |
| | TAR1-5-19 | | | | 0.3 nM | 3-10 nM (eg, |
| | homotrimer | | | | | 3 nM) |
| p55 TNFR | TAR2 | | As TAR1 monomer | As TAR1 | 500 nM to 100 pM | 500 nM to |
| | monomers | | | monomer | | 50 pM |
| | TAR2h-10 | | | | | |
| Serum | DOM7 | | 1 nM to 500 μM, | | 1 nM to 500 μM, | |
| albumin | monomers | | preferably 100 nM to | | preferably 100 nM to 10 μM | |
| | | | 10 μM | | | |
| | DOM7m-12 | | 100 nM | | | |
| | DOM7m-16 | | 200 nM | | | |
| | DOM7m-26 | | 70 nM | | | |
| | Vk Dummy | <7 mg/ml | | | | |
| | | (mutant I75N | | | | |
| | | <4 mg/ml) | | | | |
| Hen egg | HEL4 | 38 mg/ml | | | | |
| lysozyme | | (=2.95 mM) | | | | |
| | C36 | 10.5 mg/ml** | | | | |

**Higher concentrations are likely achievable through standard concentration methods.

### Example 7

### Solubility Studies on Anti-TNF-α and Anti-TNFR1 Single Immunoglobulin Variable Domains

The concentration limits achievable were examined for several different preparations of domain antibody polypeptides. Antigen specificities included human TNF-α, human TNFR1 and, as a control, hen egg lysozyme. The solubilities were evaluated for preparations of dAbs representing different formats. Solubilities were also evaluated with regard to the effect of different buffer preparations. The parameters measured were the highest concentration at which the measured concentration agreed with the calculated concentration (as measured by absorbance at 280 nm) and also the highest concentration achievable by accepting protein losses through precipitation.

### Materials

TAR1-5-19; monomeric dAb against the target antigen TNF-α; K_{d} of 30 nM; in the buffers described below:
1. TAR1-5-19 in 20 mM Na Citrate pH6.0 stock at 19.7 mg/ml;
2. TAR1-5-19 in 10 mM Potassium Phosphate pH7.4 stock at 15.8 mg/ml; and
3. TAR1-5-19 in 100 mM Glycine/200 mM Tris pH8.0 stock at 7.2 mg/ml.

HEL4; monomeric dAb against hen egg lysozyme; used as a control for high solubility. In 20 mM Na citrate/100 mM NaCl/0.01% Tween 20 pH 6.0 stock at 51.3 mg/ml.

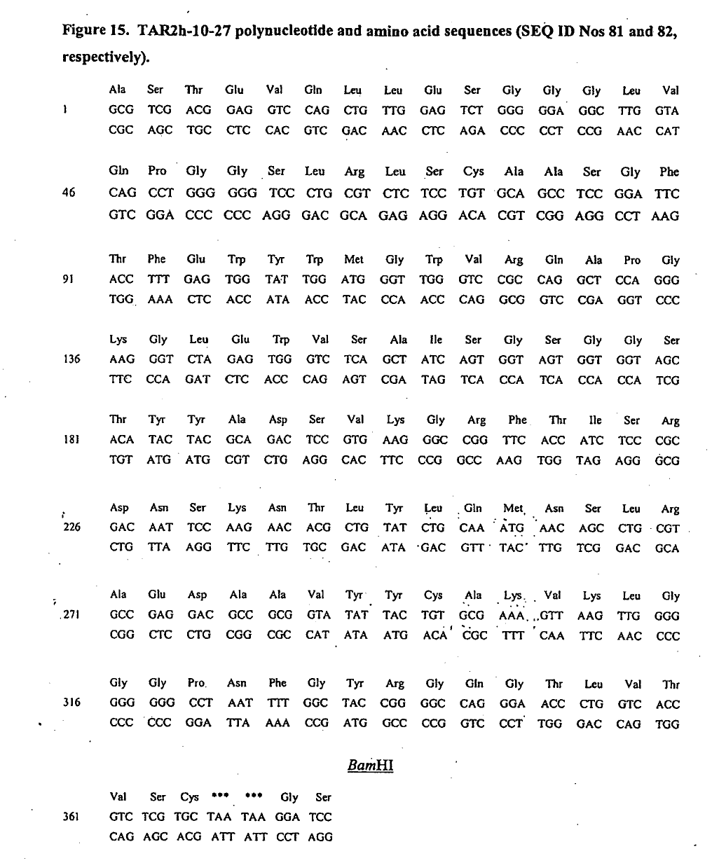

TAR2h-10-27; monomeric dAb against the target antigen TNF Receptor 1; K_{d} of 400 pM; in various formats and buffers as described below. The nucleic acid and polypeptide sequences of TAR2h-10-27 are provided in FIG. 15.
1. TAR2h-10-27cys reduced in 100 mM Glycine/Tris to pH4.0+10% glycerol at 0.75 mg/ml;
2. TAR2h-10-27 wild type, stock in Tris/Glycine pH7.0+10% glycerol at 0.06 mg/ml.
3. TAR2h-10-27cys PEGylated with 2×10K PEG in 50 mM Na Acetate pH4.0 at 0.24 mg/ml.
4. TAR2h-10-27 wild type in 100 mM Glycine/Tris to pH5.0+10% glycerol at 0.29 mg/ml.
5. TAR2h-10-27cys, reduced and alkylated with iodoacetamide in 50 mM Na Acetate pH4.0 at 0.14 mg/ml.
6. TAR2h-10-27cys in PBS pH7.2 at 1 mg/ml.

### Method

For TAR1-5-19 samples, dilutions were performed to a starting concentration of 3 mg/ml in 20 ml of respective buffer.

PBS was used to dilute the phosphate buffered TAR1-5-19, i.e. sample 2. 20 mM Na Citrate pH6.0 was used to dilute the HEL4 sample.

A280 was measured for all samples at the start of the experiment. From A280, concentration could be obtained by multiplying by 0.66 for TAR1-5-19, 0.51 for HEL4 and 0.41 for TAR2h-10-27, these correction factors being obtained from theoretical extinction coefficients.

Volumes were measured to the nearest 50 μl, using a Gilson pipette.

Samples were concentrated in 20 ml Vivaspin devices (Vivaspin AG, Germany), PES membrane, MWCO of 5 kDa. Devices were centrifuged at 3,000 g in a bench top centrifuge for 10 mins at a time at the start of the experiment, and this time interval was increased as samples became more concentrated and therefore slower to increase their concentration.

After each spin, the samples were removed from the device and the volume was measured. A 1 ml aliquot was transferred into an Eppendorf tube and spun at 16,000 g for 5 mins in a microfuge to pellet any precipitate and the A280 of 1 μl of the supernatant diluted into 100 μl buffer was measured. Aliquots were then resuspended and added back to the main pool along with the 100 μl used for A280 reading before returning it to original Vivaspin device and continuing with the concentration.

Experimental concentrations of the samples were calculated from the observed A280s and plotted vs volume measured. Also plotted were the expected concentrations for each volume, extrapolated from the starting A280, based on the linear relationship between concentration and volume i.e. C₁V₁=C₂V₂.

### Results

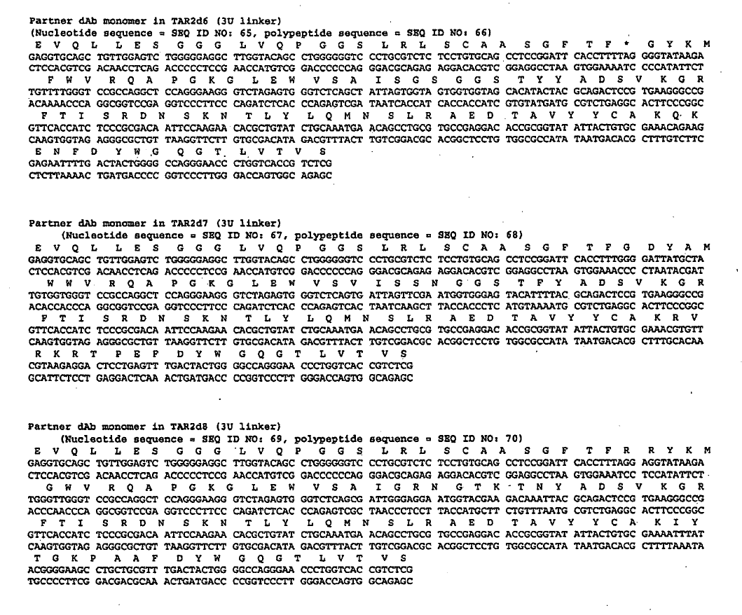

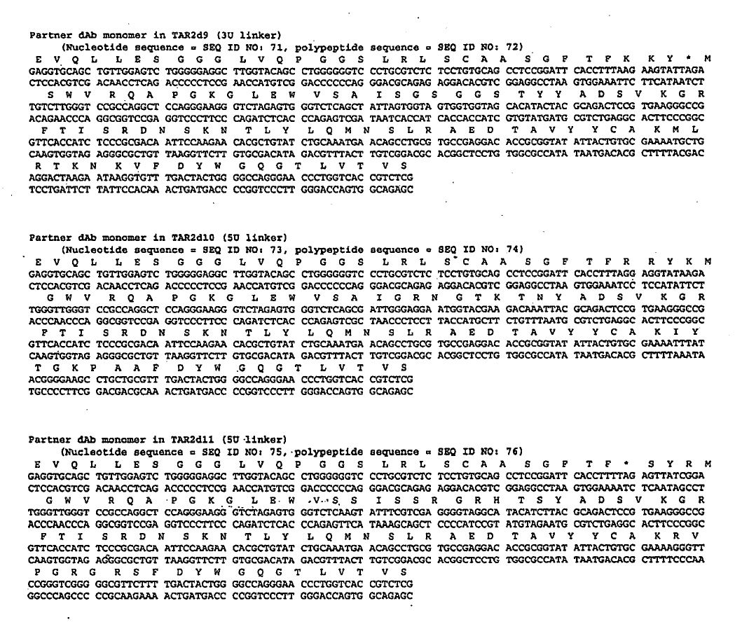

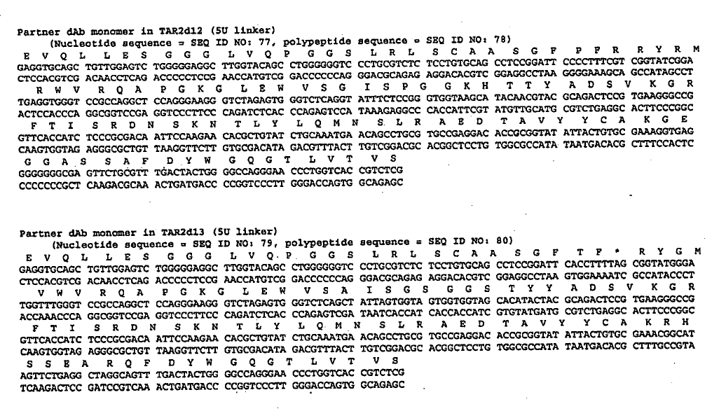

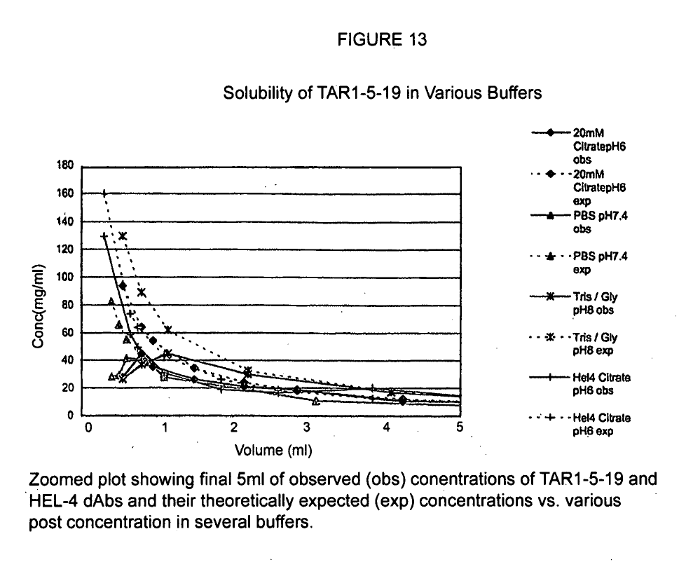

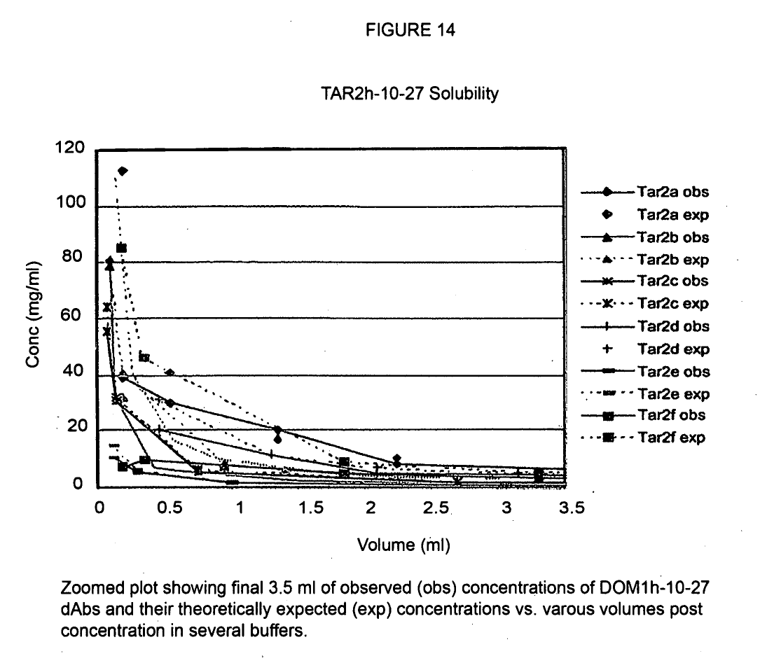

Results of these solubility studies are shown in Tables 10 (concentrations for TAR1-5-19 and HEL4) and 11 (concentrations for TAR2h-10-27) and in FIGS. 13 (TAR1-5-19) and 14 (TAR2h-10-27).

TABLE 10
Observed (obs) concentrations of TAR1-5-19 and HEL-4 dAbs and
their theoretically expected (exp) concentrations at various volumes
post concentration in several buffers.

| 20 mM | | | | | | | |
|---|---|---|---|---|---|---|---|
| CitratepH6 | | 20 mM | | | | | |
| obs | | CitratepH6 | | PBS | | PBS | |
| Vol | | exp | | pH7.4 obs | | pH7.4 exp | |
| (ml) | Conc | Vol | Conc | Vol | Conc | Vol | Conc |
| 20 | 2.574 | 20 | 2.574 | 20 | 1.65 | 20 | 1.65 |
| 15.2 | 4.488 | 15.2 | 3.386842 | 12.4 | 2.904 | 12.4 | 2.66129 |
| 11.5 | 4.224 | 11.5 | 4.476522 | 6.9 | 4.686 | 6.9 | 4.782609 |
| 8.45 | 7.458 | 8.45 | 6.092308 | 3.1 | 10.89 | 3.1 | 10.64516 |
| 5.9 | 9.702 | 5.9 | 8.725424 | 1.1 | 27.588 | 1.1 | 30 |
| 4.25 | 10.626 | 4.25 | 12.11294 | 0.85 | 40.788 | 0.85 | 38.82353 |
| 2.85 | 18.81 | 2.85 | 18.06316 | 0.6 | 41.382 | 0.6 | 55 |
| 2.15 | 20.856 | 2.15 | 23.94419 | 0.5 | 29.172 | 0.5 | 66 |
| 1.5 | 26.004 | 1.5 | 34.32 | 0.4 | 27.984 | 0.4 | 82.5 |
| 0.95 | 35.31 | 0.95 | 54.18947 | | | | |
| 0.8 | 44.814 | 0.8 | 64.35 | | | | |
| 0.55 | 27.588 | 0.55 | 93.6 | | | | |
| | | | | Hel4 | | Hel4 | |
| Tris/Gly | | Tris/Gly | | Citrate | | Citrate | |
| pH8 obs | | pH8 exp | | pH6 obs | | pH6 exp | |
| Vol | Conc | Vol | Conc | Vol | Conc | Vol | Conc |
| 20 | 3.564 | 20 | 3.564 | 20 | 2.397 | 20 | 2.397 |
| 14.6 | 3.696 | 14.6 | 4.882192 | 14.7 | 3.723 | 14.7 | 3.261224 |
| 10.3 | 7.722 | 10.3 | 6.920388 | 10.8 | 5.406 | 10.8 | 4.438889 |
| 6.8 | 8.184 | 6.8 | 10.48235 | 8 | 9.996 | 8 | 5.9925 |
| 4.1 | 17.094 | 4.1 | 17.38537 | 5.55 | 11.985 | 5.55 | 8.637838 |
| 2.2 | 29.832 | 2.2 | 32.4 | 3.85 | 20.043 | 3.85 | 12.45195 |
| 1.15 | 44.88 | 1.15 | 61.98261 | 2.6 | 16.473 | 2.6 | 18.43846 |
| 0.8 | 36.828 | 0.8 | 89.1 | 1.85 | 18.921 | 1.85 | 25.91351 |
| 0.55 | 26.268 | 0.55 | 129.6 | 1.1 | 31.059 | 1.1 | 43.58182 |
| | | | | 0.75 | 49.368 | 0.75 | 63.92 |
| | | | | 0.65 | 59.007 | 0.65 | 73.75385 |
| | | | | 0.3 | 129.4 | 0.3 | 159.8 |

TABLE 11
Observed (obs) concentrations of TAR2h-10-27 dAbs and their
theoretically expected (exp) concentrations at various volumes
post concentration in several buffers.

| TAR2h- | | TAR2h- | | TAR2h- | | TAR2h- | |
|---|---|---|---|---|---|---|---|
| 10-27(a) | | 10-27(a) | | 10-27 (b) | | 10-27(b) | |
| observed | | expected | | observed | | expected | |
| Vol (ml) | Conc | Vol | Conc | Vol | Conc | Vol | Conc |
| 30 | 0.75 | 30 | 0.75 | 135 | 0.06 | 135 | 0.060 |
| 18.85 | 1.046 | 18.85 | 1.194 | 16.45 | 0.511 | 16.45 | 0.492 |
| 11.85 | 1.15 | 11.85 | 1.899 | 7.95 | 0.921 | 7.95 | 1.019 |
| 8.1 | 2.718 | 8.1 | 2.778 | 3.95 | 2.116 | 3.95 | 2.051 |
| 5.35 | 3.998 | 5.35 | 4.206 | 1.4 | 5.715 | 1.4 | 5.786 |
| 4.4 | 4.662 | 4.4 | 5.114 | 0.95 | 7.105 | 0.95 | 8.526 |
| 2.25 | 8.171 | 2.25 | 10.000 | 0.2 | 31.693 | 0.2 | 40.5 |
| 1.35 | 20.008 | 1.35 | 16.667 | 0.1 | 78.269 | 0.1 | 81 |
| 0.55 | 29.602 | 0.55 | 40.90909 | | | | |
| 0.2 | 38.95 | 0.2 | 112.5 | | | | |

| TAR2h- | | TAR2h- | | TAR2h- | | TAR2h- | |
|---|---|---|---|---|---|---|---|
| 10-27(c) | | 10-27(c) | | 10-27(d) | | 10-27(d) | |
| obs | | exp | | obs | | exp | |
| Vol | Conc | Vol | Conc | Vol | Conc | Vol | Conc |
| 20 | 0.24 | 20 | 0.240 | 50 | 0.29 | 50 | 0.290 |
| 18.6 | 0.24 | 18.6 | 0.258 | 16.95 | 0.665 | 16.95 | 0.855 |
| 7.85 | 0.558 | 7.85 | 0.611 | 10.1 | 0.989 | 10.1 | 1.436 |
| 2.7 | 1.64 | 2.7 | 1.778 | 6.15 | 2.001 | 6.15 | 2.358 |
| 0.75 | 5.482 | 0.75 | 6.400 | 3.15 | 4.203 | 3.15 | 4.603 |
| 0.15 | 30.463 | 0.15 | 32 | 2.1 | 4.494 | 2.1 | 6.905 |
| 0.075 | 55.268 | 0.075 | 64 | 1.3 | 11.439 | 1.3 | 11.15385 |
| | | | | 0.465 | 20.664 | 0.465 | 31.1828 |

| TAR2h- | | TAR2h- | | TAR2h- | | TAR2h- | |
|---|---|---|---|---|---|---|---|
| 10-27(e) | | 10-27(e) | | 10-27(f) | | 10-27(f) | |
| obs | | exp | | obs | | exp | |
| Vol | Conc | Vol | Conc | Vol | Conc | Vol | Conc |
| 12.5 | 0.14 | 12.5 | 0.14 | 19.4 | 0.834 | 19.4 | 0.834 |
| 16.05 | 0.106 | 16.05 | 0.109034 | 15.35 | 0.911 | 15.35 | 1.054046 |
| 7.3 | 0.214 | 7.3 | 0.239726 | 13.45 | 1.123 | 13.45 | 1.202944 |
| 3.05 | 0.496 | 3.05 | 0.57377 | 12.2 | 1.263 | 12.2 | 1.326197 |
| 1 | 1.533 | 1 | 1.75 | 9 | 1.595 | 9 | 1.797733 |
| 0.3 | 5.453 | 0.3 | 5.833333 | 8.15 | 1.488 | 8.15 | 1.985227 |
| 0.12 | 10.537 | 0.12 | 14.58333 | 6.85 | 1.87 | 6.85 | 2.361985 |
| | | | | 3.3 | 3.141 | 3.3 | 4.902909 |
| | | | | 1.85 | 4.44 | 1.85 | 8.74573 |
| | | | | 0.35 | 9.717 | 0.35 | 46.22743 |
| | | | | 0.19 | 7.339 | 0.19 | 85.15579 |

Key:
TAR2h-10-27(a) = TAR2h-10-27-cys reduced in Tris/Gly + 10% glycerol pH4.
TAR2h-10-27(b) = TAR2h-10-27 wt in Tris/Gly + 10% glycerol pH7.
TAR2h-10-27(c) = TAR2h-10-27Cys PEG 2 × 10K in 50 mM Acetate pH4.
TAR2h-10-27(d) = TAR2h-10-27 wt in Tris/Gly + 10% glycerol pH5
TAR2h-10-27(e) = TAR2h-10-27Cys in 50 mM acetate, blocked i.e. non-PEGylated
TAR2h-10-27(f) = TAR2h-10-27Cys reduced in PBS pH 7.2.

### Conclusions

### A) For TAR1-5-19:

In citrate pH6: the limiting solubility appears to be ˜20 mg/ml. The maximum concentration achievable is about 40 mg/ml, but in achieving this concentration approximately 20 mg were lost in precipitation.

In PBS pH7.2: the limiting solubility appears to be ˜40 mg/ml, which is also probably the maximum concentration achievable. There were no losses to precipitation until this threshold and only then did further concentration cause precipitation.

In Tris/Gly pH8: the limiting solubility appears to be ˜30 mg/ml, with very little protein loss up to this concentration. Above this concentration, precipitation is observed. Maximum achievable concentration is ˜40 mg/ml with losses of ˜20 mg/ml.

B) For TAR2h-10-27:

TAR2h-10-27 wild type (TAR2h-10-27(b)) in buffer with glycerol agreed well with expected values. This sample had been prepared early in the project's lifetime and had thus suffered several precipitations owing to buffer incompatibility, with subsequent resuspension steps. Therefore, it is possible that all misfolded and/or unstable material was removed. It has been noted that TAR2h-10-27 displays three alternative pIs when run on an IEF gel. This suggests alternative foldings, some of which may be more soluble than others.

PEGylated TAR2h-10-27cys also agreed very well with the expected values and reached a concentration of ˜60 mg/ml with no precipitation.

Reduced TAR2h-10-27cys in PBS (DOM1 h-10-27(f)) was the most susceptible to protein loss through precipitation. The pH of PBS is close to one of the observed pI values for TAR2h-10-27.

TAR2h-10-27cys pool which had been reduced and blocked with iodoacetamide (TAR2h-10-27(e)) did not contain enough protein for any conclusion to be drawn.

At pH 4 or 5 (TAR2h-10-27(a) and TAR2h-10-27(d)), whether wild type or with C-terminal cys, the observed behaviour was similar, reaching a limiting concentration of ˜20 mg/ml or ˜10 mg/ml respectively and then precipitating out of solution. Maximum concentrations reached were 40 mg/ml and 20 mg/ml respectively, but losses of 75 mg and 10 mg of protein were required to achieve this.

### C) For HEL-4:

Concentration reached ˜130 mg/ml with protein loss measured at ˜10-15 mg, but this loss remained more or less constant throughout the experiment, suggesting possible binding to the membrane.

### Example 8

### Concentrated Preparations of Anti CD40L dAbs

dAbs specific for CD40L are referred to using the nomenclature prefix “TAR4.” Concentrated dAb preparations highly specific for CD40L were prepared using Vivaspin 5 kDa MWCO concentrators as described herein. Concentration was measured by A280.

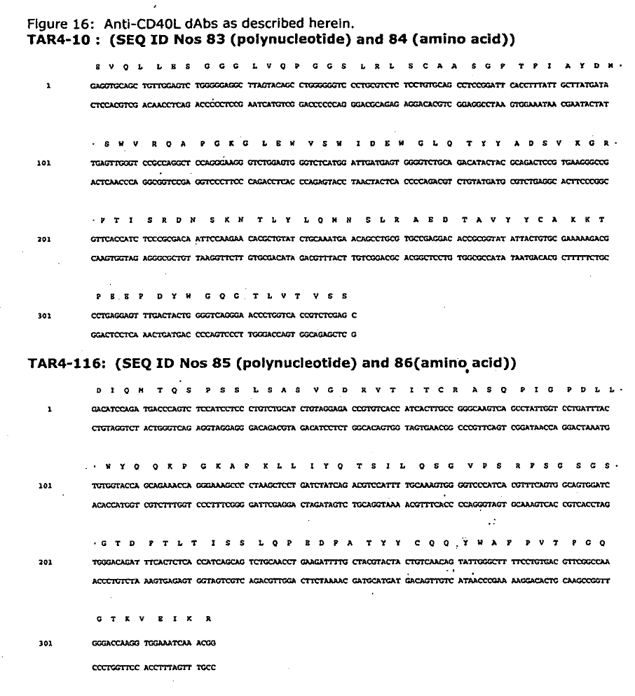

Specifically, the human CD40L-specific dAbs TAR4-10 and TAR4-116 (polynucleotide and amino acid sequences are provided in FIG. 16), which have IC50s of ˜100 nm and ˜100-250 nm, respectively, have been concentrated to 5.8 and 17.7 mg/ml in Tris-Glycine buffer, pH 8.

### Example 9

### Concentrated Preparations of PEGylated dAbs

PEGylation tends to increase the solubility of polypeptide molecules. Thus, PEGylated dAbs will generally be capable of achieving higher concentration than non-PEGylated versions of the same dAbs. However, it is important to note that the molecular weight of the PEG polymer moieties plays a role in the degree to which PEGylated dAbs can be concentrated. Large PEG polymers tend to cause the solution to become viscous, to the point where the preparations are not efficiently concentrated using centrifugal concentrators. Thus, smaller PEG polymers, e.g., 5 kDa or 10 kDa polymer, generally yields a higher end concentration than, e.g., a 30 kDa or 50 kD PEG polymer on the same dAb molecule.

As an example of the concentration achievable with a PEGylated dAb, a PEGylated version of the anti-TNFR1 dAb TAR2h-10-27, bearing linear 30 kDa PEGylation, was concentrated, using a Vivaspin concentrator, to 65 mg/ml in Tris-Acetate buffer, pH 8. Quantitation was by A₂₈₀.

It is noted that higher concentrations of PEGylated dAbs, including those with larger PEG moieties, can also be achieved by first concentrating a PEGylated dAb to the limit permitted by centrifugal concentrators, e.g., the Vivaspin 5 kDa MWCO concentrators, and then lyophilizing the remaining solution. The PEG tends to stabilize the protein to assist its solubility upon re-hydration in a smaller volume.

### Example 10

### Concentrated dAb Preparations Specific for p55 TNFR

A dAb highly specific for human p55 TNF receptor (K_{d}=10-15 nM) has been isolated and expressed from the pDOM5 vector. The amino acid sequence of the TAR2h10-55 dAb is shown below.

After expression, the TAR2h10-55 dAb was concentrated in PBS, pH 7.4 using a Vivaspin spin MWCO 3,000 Da concentrator at 4° C. and 4,000 rpm. A concentration of 88.2 mg/ml was achieved, as measured by A₂₈₀. Cell-based assays for antigen binding revealed no difference in potency of the highly concentrated dAb preparation versus non-concentrated dAb material.

| | |
|---|---|
| Amino acid sequence* of TAR2h10-55 dAb: | |
| EVQLLESGGGLVQPGGSLRLSCAASGFPFEWYWMGWVRQAPGKGLEWVSAISG | (SEQ ID NO: 87) |
| SGDSTYYADSVKGRFTISRDNSKNTLYQQMNSLRAEDAAVYYCAKVKLGGGPN | |
| FGYRGQGTLVTVSS | |

*The pDOM5 vector adds two residues (a serine-threonine dipeptide) to the N-terminus of the dAb molecules and a Myc tag (AAAEQKLISEEDLN) (SEQ ID NO: 88) to the C-terminus.

### Example 11

### Concentrated dAb Preparations Specific for Human Serum Albumin (HSA)

dAbs specific for human serum albumin have been isolated and expressed from the pDOM5 vector. Anti-serum albumin dAbs are referred to using the nomenclature prefix “TAR3” (the serum albumin binders are also referred to using the nomenclature prefix “DOM7,” e.g., in Table 9 herein). As shown in the table below, the K_{d}'s for exemplified clones TAR3h-22, TAR3h-23 and TAR3h-26 ranged from 800 nM to 50 nM. Amino acid sequences are provided below.

After expression, the HSA dAbs were concentrated in PBS, pH 7.4 using a Vivaspin spin MWCO 3,000 Da concentrator at 4° C. and 4,000 rpm. Achieved concentrations ranged from 83 to 138 mg/ml as measured by A₂₈₀. Further concentration is likely possible, as precipitation was not observed at these concentrations.

| dAb clone | IC50/Kd | Solubility (mg/ml) |
|---|---|---|
| TAR3h-22 | 50 nM | >93 |
| TAR3h-23 | 800 nM | >138 |
| TAR3h-26 | 200 nM | >90 |

| | |
|---|---|
| Amino acid sequence* of HSA dAbs: | |
| TAR3h-22 | |
| EVQLLESGGGLVQPGGSLRLSCAASGFTFSKYWMSWVRQAPGKGLEWVSSIDF | (SEQ TD NO: 89) |
| MGPHTYYADSVKGRFTISRDNSKNTLYLQMNSLRAEDTAVYYCAKGRTSMLPM | |
| KGKFDYWGQGTLVTVSS | |
| TAR3h-23 | |
| EVQLLESGGGLVQPGGSLRLSCAASGFTFYDYNMSWVRQAPGKGLEWVSTITHT | (SEQ ID NO: 90) |
| GGVTYYADSVKGRFTISRDNSKNTLYLQMNSLRAEDTAVYYCAKQNPSYQFDY | |
| WGQGTLVTVSS | |
| TAR3h-26 | |
| EVQLLESGGGLVQPGGSLRLSCTASGFTFDEYNMSWVRQAPGKGLEWVSTILPH | (SEQ ID NO: 91) |
| GDRTYYADSVKGRFTISRDNSKNTLYLQMNSLRAEDTAVYYCAKQDPLYRFDY | |
| WGQGTLVTVSS | |

*As noted above, the pDOM5 vector adds ST dipeptide to the N-terminus and a Myc tag to the C-terminus.

All patents, patent applications, and published references cited herein are hereby incorporated by reference in their entirety. While this invention has been particularly shown and described with references to preferred embodiments thereof, it will be understood by those skilled in the art that various changes in form and details may be made therein without departing from the scope of the invention encompassed by the appended claims.

1. A composition comprising a polypeptide comprising a single human immunoglobulin variable domain that binds a polypeptide antigen with a K_{d} of less than or equal to 100 nM, wherein said polypeptide is present at a concentration of at least 400 μM as determined by absorbance of light at 280 nm wavelength, wherein said polypeptide comprises a single human immunoglobulin variable domain hetero-multimer.

2. The composition of claim 1 wherein said variable domains is a V_{H} domain.

3. The composition of claim 1 wherein said variable domain is a V_{L} domain.

4. A composition comprising a polypeptide comprising a heavy chain immunoglobulin single variable domain that binds an antigen with a K_{d} of less than or equal to 100 nM, wherein said polypeptide is present at a concentration of at least 400 μM as determined by absorbance of light at 280 nm wavelength, and wherein the amino acid residue at position 44 of the variable domain is a glycine.

5. A composition comprising a polypeptide comprising a heavy chain immunoglobulin single variable domain that binds a polypeptide antigen with a K_{d} of less than or equal to 100 nM, wherein said polypeptide is present at a concentration of at least 400 μM as determined by absorbance of light at 280 nm wavelength, and wherein the amino acid residue at position 45 of the variable domain is a non-charged amino acid.

6. A composition comprising a polypeptide comprising a heavy chain immunoglobulin single variable domain that binds a polypeptide antigen with a K_{d} of less than or equal to 100 nM, wherein said polypeptide is present at a concentration of at least 400 μM as determined by absorbance of light at 280 nm wavelength, and wherein the amino acid residue at position 47 of the variable domain is a non-charged amino acid.

7. The composition of claim 4, wherein the amino acid sequence of FW2 (per Kabat numbering) of the variable domain is the same as the amino acid sequence of FW2 encoded by a human germline antibody gene segment.

8. The composition of claim 4 wherein said polypeptide consists of a human immunoglobulin V_{H} domain.

9. The composition of claim 4 wherein said antigen is a polypeptide antigen.

10. The composition of claim 2 wherein said V_{H} domain comprises the sequence encoded by germline V_{H} gene segment DP47 but which differs in sequence from that encoded by DP47 at one or more positions selected from the group consisting of H30, H31, H33, H35, H50, H52, H52a, H53, H55, H56, H58, H95, H96, H97, H98, H99, H100, H100a, H100b, H100c, H100d, H100e, and H100f.

11. The composition of claim 2 wherein said V_{H} domain comprises the sequence encoded by germline V_{H} gene segment DP47 but which differs in sequence from that encoded by DP47 at one or more positions selected from the group consisting of H30, H31, H33, H35, H50, H52, H52a, H53, H55, H56, H58, H95, H97, H98, H99, H100, H100a and H100b.

12. The composition of claim 2, wherein said V_{H} domain comprises the sequence encoded by germline V_{H} gene segment DP47 but which differs in sequence from that encoded by DP47 at one or more positions selected from the group consisting of H30, H31, H33, H35, H50, H52, H52a, H53, H55, H56, H58, H95, H97 and H98.

13. The composition of claim 4 wherein said human antigen is a human polypeptide antigen.

14. The composition of claim 4 wherein said antigen is human TNF-α or human p55 TNFR.

15. The composition of claim 1 wherein said antigen is human TNF-α or human TNFR.

16. The composition of claim 14, wherein said antigen is human TNF-α and said polypeptide neutralizes said human TNF-α in a standard L929 in vitro assay, with an IC₅₀ of 100 nM or less.

17. The composition of claim 1 wherein said polypeptide comprises a homomultimer of said single human immunoglobulin variable domain.

18. The composition of claim 17 wherein the monomers of the homomultimer are specific for a multi-subunit target.

19. The composition of claim 1 wherein the antigen target for said polypeptide is human TNF-α.

20. (canceled)

21. The composition of claim 1 wherein a monomer of said hetero-multimer comprises a single immunoglobulin variable domain polypeptide that binds serum albumin.

22. A method of treating or preventing a disease or disorder in an individual in need of such treatment, the method comprising administering a therapeutically effective amount of a composition of claim 1 to said individual.

23. The method of claim 22 wherein said human immunoglobulin single variable domain specifically binds TNF-α, p55 TNFR, EGFR, MMP-12, IgE, serum albumin, interferon gamma, CEA or PDK 1.

24. The method of claim 22 wherein said human immunoglobulin single variable domain specifically binds TNF-α or p55 TNFR.

25. A method of treating or preventing a disease or disorder in an individual in need of such treatment, the method comprising administering a therapeutically effective amount of a composition of claim 4 to said individual.

26. The method of claim 25 wherein said human immunoglobulin single variable domain specifically binds TNF-α, p55 TNFR, EGFR, MMP-12, IgE, serum albumin, interferon gamma, CEA or PDK 1.

27. The method of claim 25 wherein said human immunoglobulin single variable domain specifically binds TNF-α or p55 TNFR.

28. A composition comprising a polypeptide comprising a light chain immunoglobulin single variable domain that binds a polypeptide antigen with a K_{d} of less than or equal to 100 nM, wherein said polypeptide is present at a concentration of at least 400 .mu.M as determined by absorbance of light at 280 nm wavelength, wherein the amino acid sequence of FW2 (per Kabat numbering) of the variable domain is the same as the amino acid sequence of FW2 encoded by a human germline antibody gene segment.

29. The composition of claim 28, wherein said variable domain is a human immunoglobulin V_{L} domain.

30. The composition of claim 28, wherein the human germline gene segment is DPK9.

31. A composition comprising a polypeptide comprising a heavy chain immunoglobulin single variable domain that binds a polypeptide antigen with a K_{d} of less than or equal to 100 nM, wherein the residue at position 103 (per Kabat numbering) is an arginine, and wherein said polypeptide is present at a concentration of at least 400 μM as determined by absorbance of light at 280 nm wavelength.

32. The composition of claim 31 wherein said polypeptide antigen is a human polypeptide antigen.

33. The composition of claim 32 wherein said polypeptide antigen is human TNF-α or human TNFR.

34. The composition of claim 1 wherein the amino acid residue at position 44 of said single human immunoglobulin variable domain is a glycine.

35. The composition of claim 1 wherein the amino acid residue at position 45 is a non-charged amino acid.

36. The composition of claim 1 wherein the amino acid residue at position 45 is a leucine.

37. The composition of claim 1 wherein the amino acid residue at position 47 is a non-charged amino acid.

38. The composition of claim 1 wherein the amino acid residue at position 47 is a tryptophan.

39. The composition of claim 1 wherein the amino acid residue at position 44 is a glycine, the amino acid residue at position 45 is a leucine, and the amino acid residue at position 47 is a tryptophan.

40. The composition of claim 4 wherein the amino acid residue at position 45 is a non-charged amino acid.

41. The composition of claim 4 wherein the amino acid residue at position 45 is a leucine.

42. The composition of claim 4 wherein the amino acid residue at position 47 is a non-charged amino acid.

43. The composition of claim 4 wherein the amino acid residue at position 47 is a tryptophan.

44. The composition of claim 4 wherein the amino acid residue at position 45 is a leucine and the amino acid residue at position 47 is a tryptophan.

45. The composition of claim 1 wherein one or more of the framework regions is encoded by a human germline antibody gene segment.

46. The composition of claim 45, wherein one or more of the framework regions is encoded by human germline antibody gene segment DP47, DP45 or DP38.

47. The composition of claim 45, wherein FW3 is encoded by human germline antibody gene segment DP47.

48. The composition of claim 4 wherein one or more of the framework regions is encoded by a human germline antibody gene segment.

49. The composition of claim 48, wherein one or more of the framework regions is encoded by human germline antibody gene segment DP47, DP45 or DP38.

50. The composition of claim 48, wherein FW3 is encoded by human germine antibody gene segment DP47.

51. The composition of claim 4, wherein the amino acid sequence of one or more of said framework regions is the same as the amino acid sequence of a corresponding framework region encoded by a human germine antibody gene segment, or the amino acid sequences of one or more of said framework regions collectively comprise up to 5 amino acid differences relative to the amino acid sequence of said corresponding framework region encoded by a human germline antibody gene segment.

52. The composition of claim 4 wherein the amino acid sequences of framework regions FW1, FW2, FW3 and FW4 are the same as the amino acid sequence of corresponding framework regions encoded by a human germline antibody gene segment, or the amino acid sequences of FW1, FW2, FW3 and FW4 collectively contain up to 10 amino acid differences relative to the sequences of corresponding framework regions encoded by said human germline antibody gene segment.

53. The composition of claim 1 wherein said polypeptide is present at a concentration of 400 μM to 20 mM.

54. The composition of claims 1 wherein said polypeptide binds said antigen with a K_{d} of 100 nM to 50 pM.

55. The composition of claim 1 wherein said polypeptide binds said antigen with a K_{d} of 30 nM to 50 pM.

56. The composition of claim 1 that further comprises a pharmaceutically acceptable carrier.

57. The composition of claim 1 wherein said immunoglobulin variable domain is coupled to a polymer which comprises a substituted or unsubstituted straight or branched chain polyalkylene, polyalkenylene or polyoxyalkylene polymer or a branched or unbranched polysaccharide.

58. An extended release dosage formulation comprising a composition of claim 1.

59. The extended release dosage formulation of claim 58 which is formulated for oral or parenteral administration.

60. The extended release dosage formulation of claim 59 wherein said dosage formulation is provided for parenteral administration via a route selected from the group consisting of intravenous, intramuscular or intraperitoneal injection, implantation, rectal and transdermal administration.

61. The extended release dosage formulation of claim 60 wherein said implantation comprises intratumor implantation.

62. A method of treating a disease or disorder comprising administering the extended release dosage formulation of claim 58.
